(19) United States
(12) Patent Application Publication
(10) Pub. No.: US 2008/0085893 A1
(43) Pub. Date: Apr. 10, 2008

(54) MATRIX METALLOPROTEASE INHIBITORS

(76) Inventors: Shyh-Ming YANG, High Bridge, NJ (US); Bingbing Wang, Flemington, NJ (US); Robert Scannevin, Doylestown, PA (US); Kenneth Rhodes, Doylestown, PA (US); Bharat Lagu, Hillsborough, NJ (US); Lawrence J. Wilson, Atlanta, GA (US); Prabha Karnachi, Hillsborough, NJ (US); William V. Murray, Belle Mead, NJ (US)

Correspondence Address: PHILIP S. JOHNSON;JOHNSON & JOHNSON, ONE JOHNSON & JOHNSON PLAZA, NEW BRUNSWICK, NJ, 08933-7003, US

(21) Appl. No.: 11/859,910

(22) Filed: Sep. 24, 2007

Related U.S. Application Data
(60) Provisional application No. 60/849,854, filed on Oct. 6, 2006.

(51) Int. Cl. A61K 31/18; A61K 31/351; A61K 31/381; A61K 31/404; A61K 31/4166; A61K 31/4409; A61K 31/4412; A61K 31/45; A61K 31/472; A61K 31/495; A61K 31/5375; A61K 31/54; A61P 25/00; A61P 35/00; C07C 311/08; C07D 209/08; C07D 209/44; C07D 211/46; C07D 213/56; C07D 213/64; C07D 217/04; C07D 233/02; C07D 241/04; C07D 279/12; C07D 295/15; C07D 333/24; C07D 413/02; C12N 9/99

(52) U.S. Cl. 514/227.5; 435/184; 514/235.5; 514/238.2; 514/255.01; 514/255.02; 514/255.03; 514/307; 514/327; 514/351; 514/357; 514/389; 514/415; 514/416; 514/438; 514/451; 514/605; 544/131; 544/160; 544/383; 544/389; 544/393; 544/58.2; 546/146; 546/221; 546/300; 546/337; 548/319.5; 548/482; 548/510; 549/77; 564/99

(57) ABSTRACT

The present invention relates to compounds of Formula I, 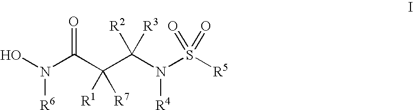 wherein R1, R2, R3, R4, R5, R6, and R7 are defined in the specification. In addition, the present invention relates to methods treating disorders related to matrix metalloproteases. More particularly, the compounds of the present invention are useful for treating stroke.

This application claims the benefit under 35 U.S.C. 119(e) of U.S. Provisional application Ser. No. 60/849,854 filed Oct. 6, 2006. The complete disclosure of the aforementioned related U.S. patent application is hereby incorporated herein by reference for all purposes.

### FIELD OF THE INVENTION

This invention relates generally to the field of matrix metalloproteinase (MMP) inhibitors and their therapeutic and prophylactic uses. Examples of relevant therapeutic area include inflammation, oncology, cardiovascular disease, and neurological disorders. More specifically, they have utility in the treatment and prevention of stroke.

### BACKGROUND OF THE INVENTION

Matrix metalloproteinases (MMPs) are a family of structurally related zinc-dependent proteolytic enzymes that digest extracellular matrix proteins such as collagen, elastin, laminin and fibronectin. Currently, at least 28 different mammalian MMP proteins have been identified, and are grouped based on substrate specificity and domain structure. The wide variety of MMPs normally participate in many different homeostatic tissue remodeling events. Given this broad functional diversity it is not surprising that MMP dysfunction would give rise to a host of different pathologies. A role for MMPs in oncology has been the most extensively explored, as up-regulation of any number of MMPs are one mechanism by which malignant cells can overcome connective tissue barriers and metastasize (Curr Cancer Drug Targets 5: 203-20 (2005)). MMPs also appear to have a direct role in angiogenesis, also making them an important target for oncology indications (Int J Cancer 115: 849-60 (2005) and J Cell Mol Med 9: 267-85 (2005)). Several different classes of MMPs are involved in these processes, but MMP-2, -9 and MT1-MMP have been most often implicated. The cartilage and bone degeneration that results in osteoarthritis and rheumatoid arthritis are due primarily to MMP digestion of the ECM in bone and joints (Aging Clin Exp Res 15: 364-72 (2003) and Joint Bone Spine (2005)). MMP-1, -3, -9, and -13 have been found to be elevated in the tissues and body fluids surrounding damaged areas. MMPs also have a role in cardiovascular diseases, in that they are believed to be involved in atherosclerotic plaque rupture, aneurysm and vascular and myocardial tissue morphogenesis (Expert Opin Investig Drugs 9: 993-1007 (2000) and Curr Med Chem 12: 917-25 (2005)). Elevated levels of MMP-1, -2, -9, and -13 have often been associated with these conditions. Several other pathologies such as gastric ulcers, pulmonary hypertension, chronic obstructive pulmonary disease, inflammatory bowel disease, periodontal disease, skin ulcers, liver fibrosis, emphysema, and Marfan syndrome appear to have a MMP component as well (Expert Opin Ther Patents 12: 665-707 (2002)).

Within the central nervous system, altered MMP expression has been linked to several neurodegenerative disease states (Expert Opin Investig Drugs 8: 255-68 (1999)), most notably in stroke (Glia 50: 329-39 (2005)). Two enzymes, MMP-2 and MMP-9, appear to have the most significant impact in propagating the brain tissue damage that occurs following an ischemic or hemorrhagic insult. Studies in human stroke patients and in animal stroke models have demonstrated that both MMP-2 and -9 expression levels and activity increase sharply over a 24 hour period following an ischemic event. Within the brain, the microvascular endothelial cell tight-junctions are broken down by activated MMP-2 and -9, which results in increased permeability of the blood-brain barrier (BBB). This breakdown in the integrity of the BBB then leads to edema and infiltration of inflammatory agents, both of which cause increased cell death around the infarct core (the penumbra) and increase the possibility of hemorrhagic transformation. Administration of MMP inhibitors has shown to be protective in animal models of stroke (Stroke 29: 1020-30 (1998), Expert Opin Investig Drugs 8: 255-68 (1999), Stroke 31: 3034-40 (2000), Stroke 34: 2025-30 (2003) and J Neurosci 25: 6401-8 (2005)). MMP-9 knockout animals also demonstrate significant neuroprotection in similar stroke models (J Cereb Blood Flow Metab 20: 1681-9 (2000)). In the US, stroke is the third leading cause of mortality, and the leading cause of disability. Currently, thrombolitics (e.g. t-PA) are the only approved therapy for stroke, however its use is severely limited due to a narrow dosing window and potentially dangerous safety profile. This area has a large unmet medical need for acute interventional therapy.

MMP-9 has also been suggested to play a role in the progression of multiple sclerosis (MS). Studies have indicated that serum levels of MMP-9 are elevated in active patients, and are concentrated around MS lesions (Lancet Neurol 2: 747-56 (2003)). Increased serum MMP-9 activity would promote infiltration of leukocytes into the CNS, a causal factor and one of the hallmarks of the disease. MMPs may also contribute to severity and prolongation of migraines. In animal models of migraine (cortical spreading depression), MMP-9 is rapidly upregulated and activated leading to a breakdown in the BBB, which results in mild to moderate edema (J Clin Invest 113: 1447-55 (2004)). It is this brain swelling and subsequent vasoconstriction which causes the debilitating headaches and other symptoms associated with migraine. In the cortical spreading depression model, MMP inhibitors have been shown to prevent the opening of the BBB (J Clin Invest 113: 1447-55 (2004)). Related research has shown that MMP-9 is specifically upregulated in damaged brain tissues following traumatic brain injury (J Neurotrauma 19: 615-25 (2002)), which would be predicted to lead to further brain damage due to edema and immune cell infiltration. MMPs may also have additional roles in additional chronic CNS disorders. In an animal model of Parkinson's disease, MMP-9 was found to be rapidly upregulated after striatal injection of a dopaminergic neuron poison (MPTP) (Neuromolecular Med 5: 119-32 (2004)), and MMP-3 has been shown to process α-synuclein to an aggregation-prone form (J Biol Chem 280: 25216-24 (2005)). This implicates MMPs in both the neuronal remodeling that occurs upon cell loss and one of the potential causative factors of the disease. In patients with Alzheimer's disease, MMP-9 was found to be upregulated in postmortem plasma samples compared to normal controls (Expert Opin Investig Drugs 8: 255-68 (1999) and Neurochem Int 43: 191-6 (2003)). Furthermore, pathologic expression of Aβ peptides induces expression and activation of MMP-2, which may contribute to cerebral amyloid angiopathy, a major pathological feature of Alzheimer's disease (J Neurochem 85: 1208-15 (2003)). MMPs may also have a role in vascular dementia, as MMP-9 levels have been found to be elevated in the cerebrospinal fluid from demented patients (Stroke 35: e159-62 (2004)). Clearly, the pathologic expression of various MMPs can contribute to many different neurodegenerative disorders.

### SUMMARY OF THE INVENTION

This invention relates, in part, to methods and compositions useful for the treatment of matrix metalloproteinase-mediated conditions. Specifically, in part, the invention relates to compounds of Formula I: 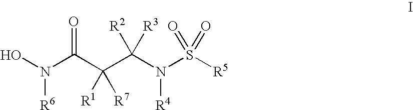
wherein:
R¹ is H, —CH₃;
R² is H, —CH₃;
R³ is H, C₍₁₋₃₎alkyl, phenyl, 5- or 6-membered heteroaryl;
   wherein said phenyl and said 5- or 6-membered heteroaryl are optionally substituted with one or two substituents selected from the group consisting of Cl, F, —N(C₍₁₋₄₎alkyl)₂, —NO₂, —CN, —OCF₃, —CF₃, —OC₍₁₋₄₎alkyl, and C₍₁₋₄₎alkyl; 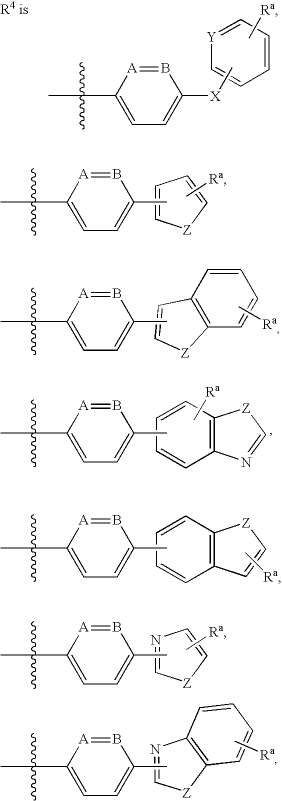 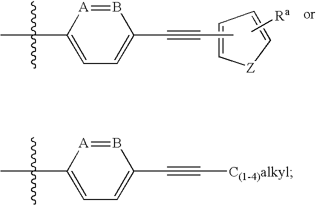
      wherein:
   A=B is —CH═CH— or S;
   X is O, S, 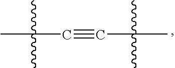
   —CH═CH— (cis or trans), or a direct bond;
   Y is CH or N;
   Z is S or O or NH
   R^{a} is one or two substituents independently selected from the group consisting of H, Cl, F, —OH, —NH₂, —N(C₍₁₋₄₎alkyl)₂, —NO₂, —CN, —CO₂H, —CONH₂, —OCF₃, —CF₃, —OC₍₁₋₄₎alkyl, and C₍₁₋₄₎alkyl;
R⁵ is C₍₁₋₄₎alkyl, phenyl, —C₍₁₋₃₎alkyl-phenyl, —C₍₁₋₃₎alkyl-heteroaryl, —N(C₍₁₋₄₎alkyl)₂, 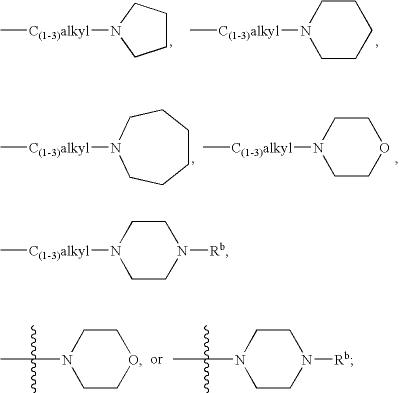
   wherein said phenyl, —C₍₁₋₃₎alkyl-phenyl, and —C₍₁₋₃₎alkyl-heteroaryl are optionally substituted with one or two substituents selected from the group consisting of Cl, F, —N(C₍₁₋₄₎alkyl)₂, —NO₂, —CN, —CONH₂, —OCF₃, —CF₃, —OC₍₁₋₄₎alkyl, and C₍₁₋₄₎alkyl;
   and R^{b} is —C₍₁₋₄₎alkyl, —SO₂C₍₁₋₄₎alkyl, —COC₍₁₋₄₎alkyl, or —CO₂CH₂-phenyl;
R⁶ is H, C₍₁₋₄₎alkyl, allyl, —C₍₂₋₄₎alkyl-O(C₍₁₋₄₎alkyl), —C₍₁₋₃₎alkyl-phenyl, —C₍₁₋₃₎alkyl-heteroaryl, —C₍₂₋₄₎alkyl-N(C₍₁₋₄₎alkyl)₂, 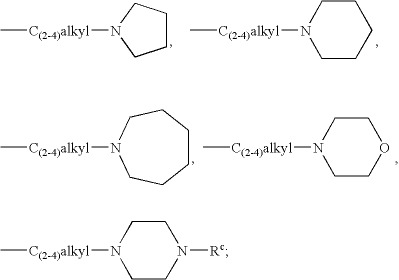
   wherein said —C₍₁₋₃₎alkyl-phenyl and said —C₍₁₋₃₎alkyl-heteroaryl are optionally substituted with one or two substituents selected from the group consisting of Cl, F, —N(C₍₁₋₄₎alkyl)₂, —NO₂, —CN, —CONH₂, —OCF₃, —CF₃, —OC₍₁₋₄₎alkyl, and C₍₁₋₄₎alkyl;
   wherein R^{c} is —C₍₁₋₄₎alkyl, —SO₂C₍₁₋₄₎alkyl, —COC₍₁₋₄₎alkyl, —CO₂CH₂-phenyl;
R⁷ is —OR⁸—SR⁸—NR⁹R¹⁰, or —NR¹¹COR¹²
   wherein R⁸ is H, C₍₁₋₄₎alkyl, phenyl, heteroaryl, indolyl, C₍₁₋₂₎alkyl-phenyl, C₍₁₋₂₎alkyl-indolyl, or C₍₁₋₂₎alkyl-heteroaryl; wherein said phenyl, heteroaryl, indolyl, C₍₁₋₂₎alkyl-phenyl, C₍₁₋₂₎alkyl-indolyl, and C₍₁₋₂₎alkyl-heteroaryl are optionally substituted with one or two substituents selected from the group consisting of Cl, F, —N(C₍₁₋₄₎alkyl)₂, —NO₂, —CN, —CONH₂, —OCF₃, —CF₃, —OC₍₁₋₄₎alkyl, and C₍₁₋₄₎alkyl;
   R⁹ is H, C₍₁₋₄₎alkyl, phenyl, heteroaryl, benzofused-heteroaryl, napthalenyl, C₍₃₋₇₎cycloalkyl, 5-7-membered heterocyclyl, C₍₂₋₄₎alkyl-carbamic acid benzyl ester, —C₍₂₋₄₎alkyl-1,5,5-trimethyl-imidazolidine-2,4-dione, C₍₁₋₃₎alkyl-phenyl, C₍₁₋₃₎alkyl-heteroaryl, C₍₁₋₃₎alkyl-benzofused-heteroaryl, and wherein said C₍₁₋₃₎alkyl-phenyl, C₍₁₋₃₎alkyl-heteroaryl, C₍₁₋₃₎alkyl-benzofused-heteroaryl, phenyl, heteroaryl, and said bezo-fused heteroaryl are optionally substituted with one or two substituents selected from the group consisting of Cl, F, —N(C₍₁₋₄₎alkyl)₂, —NO₂, —CN, —CONH₂, —OCF₃, —CF₃, —OC₍₁₋₄₎alkyl, and C₍₁₋₄₎alkyl;
   R¹⁰ is H, or C₍₁₋₄₎alkyl; alternatively, R⁹ and R¹⁰ may join together with the attached nitrogen to form a ring selected from the group consisting 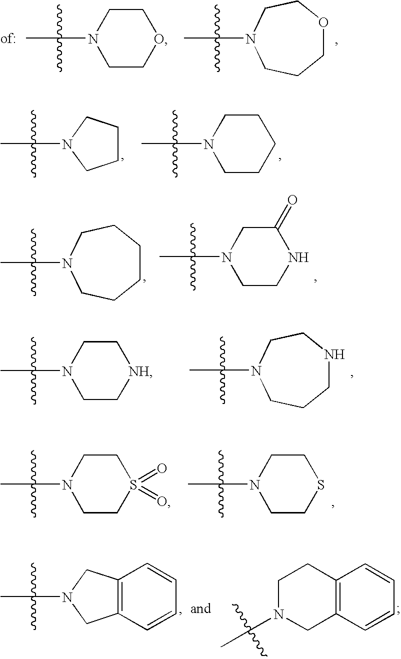
   wherein said ring is optionally substituted with one or two substituents selected from the group consisting of: —CH₂OC₍₁₋₂₎alkyl, —N(C₍₁₋₄₎alkyl)₂, —OC₍₁₋₄₎alkyl, C₍₁₋₄₎alkyl, —OH, —SO₂C₍₁₋₄₎alkyl, —COC₍₁₋₄₎alkyl, —CONHC₍₁₋₄₎alkyl, phenyl, fluorophenyl, chlorophenyl, bromophenyl, and —CO₂CH₂phenyl.
   R¹¹ is H, or C₍₁₋₄₎alkyl;
   R¹² is C₍₁₋₄₎alkyl, indolyl, phenyl, heteroaryl, C₍₃₋₇₎cycloalkyl, morpholinyl, piperidinyl, piperazinyl, N-methyl piperazinyl, tetrahydropyranyl, pyrrolidinyl, C₍₁₋₂₎alkyl-phenyl, C₍₁₋₂₎alkyl-indolyl or C₍₁₋₂₎alkyl-heteroaryl; wherein said indolyl, phenyl, heteroaryl, C₍₁₋₂₎alkyl-phenyl, C₍₁₋₂₎alkyl-indolyl or C₍₁₋₂₎alkyl-heteroaryl are optionally substituted with one or two substituents selected from the group consisting of Cl, F, —N(C₍₁₋₄₎alkyl)₂, —NO₂, —CN, —CONH₂, —OCF₃, —CF₃, —OC₍₁₋₄₎alkyl, and C₍₁₋₄₎alkyl;
      and solvates, hydrates, tautomers and pharmaceutically acceptable salts thereof.

In addition, this invention relates, in part, to methods of treating a condition that can be ameliorated by antagonizing matrix metalloproteinases, which condition includes, but is not limited to, vascular and myocardial tissue morphogenesis, cancer, cardiovascular disease, acute and chronic CNS disorders, neurodegenerative diseases, and movement disorder.

In one aspect, this invention provides a method of therapeutic and prophylactic uses of compounds of Formula I for one or more conditions selected from ischemic or hemorrhagic insult such as Parkinson's disease, Alzheimer's disease, cerebral amyloid angiopathy, vascular dementia, stroke, headache such as migraine, traumatic brain injury, edema, atherosclerotic plaque rupture, aneurysm, osteoarthritis, rheumatoid arthritis, multiple sclerosis, gastric ulcers, pulmonary hypertension, chronic obstructive pulmonary disease, inflammatory bowel disease, periodontal disease, skin ulcers, liver fibrosis, emphysema, Marfan syndrome, and associated symptoms or complications thereof. A preferred indication is stroke.

### DETAILED DESCRIPTION OF THE INVENTION

Specifically, in part, the invention relates to compounds of Formula I: 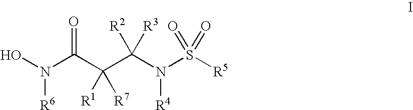
wherein:
R¹ is H, —CH₃;
R² is H, —CH₃;
R³ is H, C₍₁₋₃₎alkyl (including —CH₃), phenyl, 5- or 6-membered heteroaryl;
wherein said phenyl and said 5- or 6-membered heteroaryl are optionally substituted with one or two substituents selected from the group consisting of Cl, F, —N(C₍₁₋₄₎alkyl)₂, —NO₂, —CN, —OCF₃, —CF₃, —OC₍₁₋₄₎alkyl, and C₍₁₋₄₎alkyl; 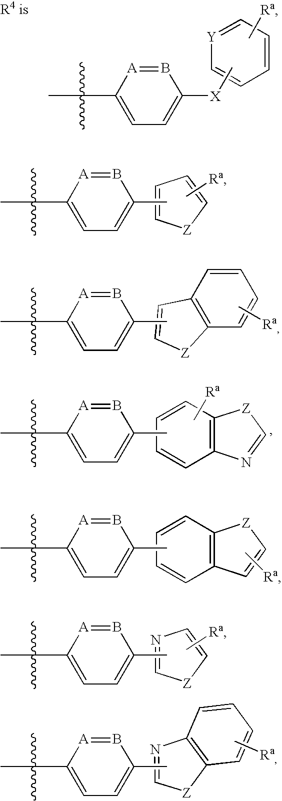 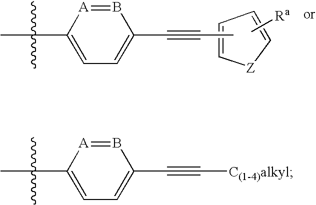
wherein:
A=B is —CH═CH— or S;
X is O, S, 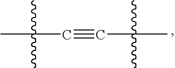
—CH═CH— (cis or trans), or a direct bond;
Y is CH or N;
Z is S or O or NH
R^{a} is one or two substituents independently selected from the group consisting of H, Cl, F, —OH, —NH₂, —N(C₍₁₋₄₎alkyl)₂, —NO₂, —CN, —CO₂H, —CONH₂, —OCF₃, —CF₃, —OC₍₁₋₄₎alkyl (including —OCH₃), and C₍₁₋₄₎alkyl (including —CH₃);
   R⁵ is C₍₁₋₄₎alkyl (including C₍₁₋₃₎alkyl), phenyl, —C₍₁₋₃₎alkyl-phenyl, —C₍₁₋₃₎alkyl-heteroaryl, —N(C₍₁₋₄₎alkyl)₂ (including —N(CH₃)₂), 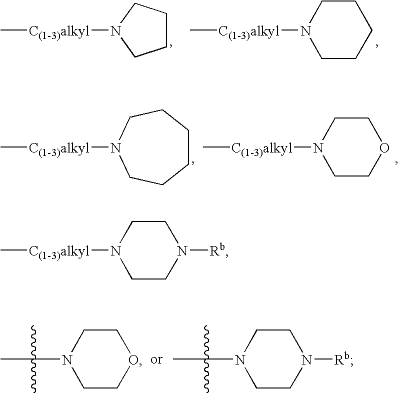
wherein said phenyl, —C₍₁₋₃₎alkyl-phenyl, and —C₍₁₋₃₎alkyl-heteroaryl are optionally substituted with one or two substituents selected from the group consisting of Cl, F, —N(C₍₁₋₄₎alkyl)₂, —NO₂, —CN, —CONH₂, —OCF₃, —CF₃, —OC₍₁₋₄₎alkyl, and C₍₁₋₄₎alkyl;
and R^{b} is —C₍₁₋₄₎alkyl, —SO₂C₍₁₋₄₎alkyl, —COC₍₁₋₄₎alkyl, or —CO₂CH₂-phenyl;
   R⁶ is H, C₍₁₋₄₎alkyl, allyl, —C₍₂₋₄₎alkyl-O(C₍₁₋₄₎alkyl), —C₍₁₋₃₎alkyl-phenyl, —C₍₁₋₃₎alkyl-heteroaryl, —C₍₂₋₄₎alkyl-N(C₍₁₋₄₎alkyl)₂, 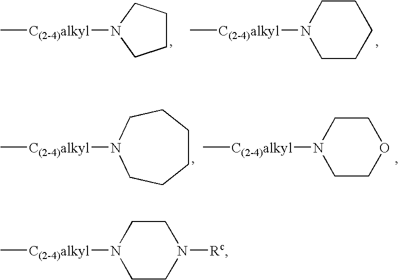
wherein said —C₍₁₋₃₎alkyl-phenyl and said —C₍₁₋₃₎alkyl-heteroaryl are optionally substituted with one or two substituents selected from the group consisting of Cl, F, —N(C₍₁₋₄₎alkyl)₂, —NO₂, —CN, —CONH₂, —OCF₃, —CF₃, —OC₍₁₋₄₎alkyl, and C₍₁₋₄₎alkyl;
wherein R^{c} is —C₍₁₋₄₎alkyl, —SO₂C₍₁₋₄₎alkyl, —COC₍₁₋₄₎alkyl, —CO₂CH₂-phenyl;
   R⁷ is —OR⁸—SR⁸—NR⁹R¹⁰, or —NR¹¹COR¹²
wherein R³ is H, C₍₁₋₄₎alkyl, phenyl, heteroaryl (including pyridyl), indolyl, C₍₁₋₂₎alkyl-phenyl, C₍₁₋₂₎alkyl-indolyl, or C₍₁₋₂₎alkyl-heteroaryl; wherein said phenyl, heteroaryl, indolyl, C₍₁₋₂₎alkyl-phenyl, C₍₁₋₂₎alkyl-indolyl, and C₍₁₋₂₎alkyl-heteroaryl are optionally substituted with one or two substituents selected from the group consisting of Cl, F, —N(C₍₁₋₄₎alkyl)₂, —NO₂, —CN, —CONH₂, —OCF₃, —CF₃, —OC₍₁₋₄₎alkyl, and C₍₁₋₄₎alkyl (including C₍₁₋₃₎alkyl);
R⁹ is H, C₍₁₋₄₎alkyl, phenyl, heteroaryl, benzofused-heteroaryl, napthalenyl, C₍₃₋₇₎cycloalkyl, 5-7-membered heterocyclyl, C₍₂₋₄₎alkyl-carbamic acid benzyl ester, —C₍₂₋₄₎alkyl-1,5,5-trimethyl-imidazolidine-2,4-dione, C₍₁₋₃₎alkyl-phenyl, C₍₁₋₃₎alkyl-heteroaryl, C₍₁₋₃₎alkyl-benzofused-heteroaryl, and wherein said C₍₁₋₃₎alkyl-phenyl, C₍₁₋₃₎alkyl-heteroaryl, C₍₁₋₃₎alkyl-benzofused-heteroaryl, phenyl, heteroaryl, and said bezo-fused heteroaryl are optionally substituted with one or two substituents selected from the group consisting of Cl, F, —N(C₍₁₋₄₎alkyl)₂, —NO₂, —CN, —CONH₂, —OCF₃, —CF₃, —OC₍₁₋₄₎alkyl (including methoxy), and C₍₁₋₄₎alkyl;
R¹⁰ is H, or C₍₁₋₄₎alkyl (including C₍₁₋₃₎alkyl); alternatively, R⁹ and R¹⁰ may join together with the attached nitrogen to form a ring selected from the group consisting of: 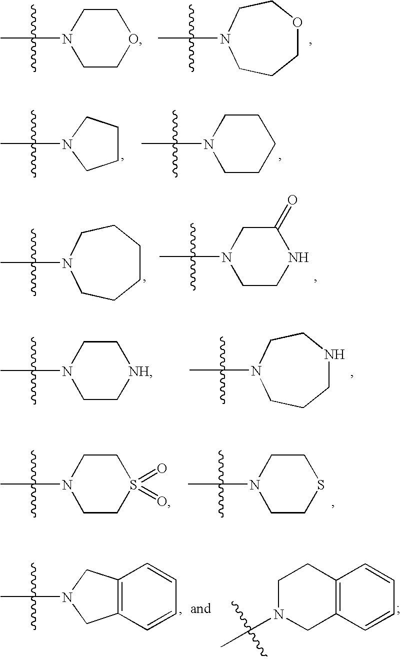
wherein said ring is optionally substituted with one or two substituents selected from the group consisting of: —CH₂OC₍₁₋₂₎alkyl, —N(C₍₁₋₄₎alkyl)₂ (including —N(C₍₁₋₃₎alkyl)₂), —OC₍₁₋₄₎alkyl (including —OC₍₁₋₃₎alkyl), C₍₁₋₄₎alkyl (including C₍₁₋₃₎alkyl), —OH, —SO₂C₍₁₋₄₎alkyl (including —SO₂CH₃), —COC₍₁₋₄₎alkyl (including —COC₍₁₋₃₎alkyl), —CONHC₍₁₋₄₎alkyl (including —CONHC₍₁₋₃₎alkyl), phenyl, fluorophenyl, chlorophenyl, bromophenyl, and —CO₂CH₂phenyl.
R¹¹ is H, or C₍₁₋₄₎alkyl;
R¹² is C₍₁₋₄₎alkyl, indolyl, phenyl, heteroaryl, C₍₃₋₇₎cycloalkyl (including C₍₅₋₆₎cycloalkyl), morpholinyl, piperidinyl, piperazinyl, N-methyl piperazinyl, tetrahydropyranyl, pyrrolidinyl, C₍₁₋₂₎alkyl-phenyl, C₍₁₋₂₎alkyl-indolyl or C₍₁₋₂₎alkyl-heteroaryl; wherein said indolyl, phenyl, heteroaryl, C₍₁₋₂₎alkyl-phenyl, C₍₁₋₂₎alkyl-indolyl or C₍₁₋₂₎alkyl-heteroaryl are optionally substituted with one or two substituents selected from the group consisting of Cl, F, —N(C₍₁₋₄₎alkyl)₂, —NO₂, —CN, —CONH₂, —OCF₃, —CF₃, —OC₍₁₋₄₎alkyl, and C₍₁₋₄₎alkyl;
   and solvates, hydrates, tautomers and pharmaceutically acceptable salts thereof.

In a preferred embodiment of the invention:

R¹ is H, or —CH₃;

R² is H, or —CH₃;

R³ is H, phenyl, or —CH₃;

wherein said phenyl is optionally substituted with one substituent selected from the group consisting of Cl, F, —N(C₍₁₋₄₎alkyl)₂, —OCF₃, —CF₃, —OC₍₁₋₄₎alkyl, and C₍₁₋₄₎alkyl; 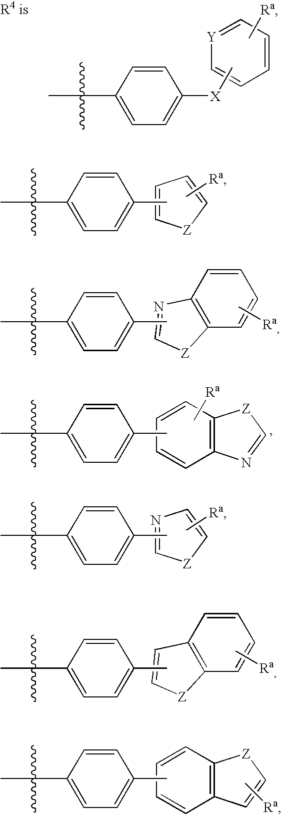 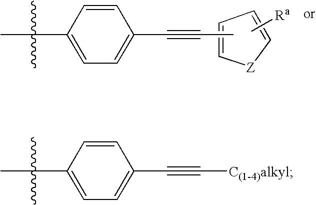
wherein:
X is O, S, 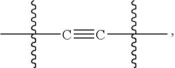
—CH═CH— (cis or trans), or a direct bond;
Y is CH or N;
Z is S or O or NH
R^{a} is one or two substituents independently selected from the group consisting of H, Cl, F, —OH, —NH₂, —N(C₍₁₋₄₎alkyl)₂, —NO₂, —CN, —CO₂H, —CONH₂, —OCF₃, —CF₃, —OC₍₁₋₄₎alkyl, and C₍₁₋₄₎alkyl;
   R⁵ is C₍₁₋₄₎alkyl, phenyl, —C₍₁₋₃₎alkyl-phenyl, —C₍₁₋₃₎alkyl-heteroaryl, —N(C₍₁₋₄₎alkyl)₂, 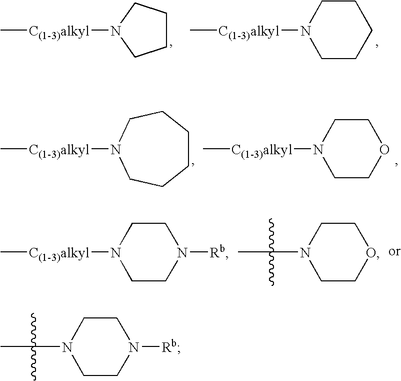
wherein said phenyl, —C₍₁₋₃₎alkyl-phenyl, and —C₍₁₋₃₎alkyl-heteroaryl are optionally substituted with one or two substituents selected from the group consisting of Cl, F, —N(C₍₁₋₄₎alkyl)₂, —OCF₃, —CF₃, —OC₍₁₋₄₎alkyl, and C₍₁₋₄₎alkyl;
and R^{b} is —C₍₁₋₄₎alkyl, —SO₂C₍₁₋₄₎alkyl, —COC₍₁₋₄₎alkyl, or —CO₂CH₂-phenyl;
   R⁶ is H, C₍₁₋₄₎alkyl, allyl, —C₍₂₋₄₎alkyl-O(C₍₁₋₄₎alkyl), —C₍₁₋₃₎alkyl-phenyl, —C₍₁₋₃₎alkyl-heteroaryl, —C₍₂₋₄₎alkyl-N(C₍₁₋₄₎alkyl)₂, 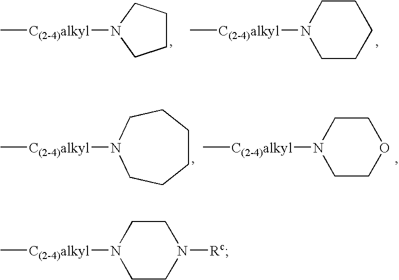
wherein said C₍₁₋₃₎alkyl-phenyl and said —C₍₁₋₃₎alkyl-heteroaryl are optionally substituted with one or two substituents selected from the group consisting of Cl, F, —N(C₍₁₋₄₎alkyl)₂, —OCF₃, —CF₃, —OC₍₁₋₄₎alkyl, and C₍₁₋₄₎alkyl;
wherein R^{c} is —C₍₁₋₄₎alkyl, —SO₂C₍₁₋₄₎alkyl, —COC₍₁₋₄₎alkyl, —CO₂CH₂-phenyl;
   R⁷ is —OR⁸, —SR⁸—NR⁹R¹⁰, or —NR¹¹COR¹²
wherein R⁸ is H, C₍₁₋₄₎alkyl, phenyl, indolyl, pyridyl, C₍₁₋₂₎alkyl-phenyl, C₍₁₋₂₎alkyl-furanyl, C₍₁₋₂₎alkyl-pyrrolyl, C₍₁₋₂₎alkyl-indolyl, or C₍₁₋₂₎alkyl-pyridyl; wherein said phenyl, C₍₁₋₂₎alkyl-phenyl, C₍₁₋₂₎alkyl-furanyl, C₍₁₋₂₎alkyl-pyrrolyl, C₍₁₋₂₎alkyl-indolyl, and C₍₁₋₂₎alkyl-pyridyl are optionally substituted with one or two substituents selected from the group consisting of Cl, F, —OCF₃, —CF₃, —OC₍₁₋₄₎alkyl, and C₍₁₋₄₎alkyl;
R⁹ is H, C₍₁₋₄₎alkyl, phenyl, heteroaryl, benzofused-heteroaryl, napthalenyl, C₍₃₋₇₎cycloalkyl, C₍₂₋₄₎alkyl-carbamic acid benzyl ester, —C₍₂₋₄₎alkyl-1,5,5-trimethyl-imidazolidine-2,4-dione, C₍₁₋₃₎alkyl-phenyl, C₍₁₋₃₎alkyl-heteroaryl, C₍₁₋₃₎alkyl-benzofused-heteroaryl, and wherein said C₍₁₋₃₎alkyl-phenyl, C₍₁₋₃₎alkyl-heteroaryl, C₍₁₋₃₎alkyl-benzofused-heteroaryl, phenyl, heteroaryl, and said bezo-fused heteroaryl are optionally substituted with one or two substituents selected from the group consisting of Cl, F, —OCF₃, —CF₃, —OC₍₁₋₄₎alkyl, and C₍₁₋₄₎alkyl;
R¹⁰ is H, or C₍₁₋₄₎alkyl; alternatively, R⁹ and R¹⁰ may join together with the attached nitrogen to form a ring selected from the group consisting of: 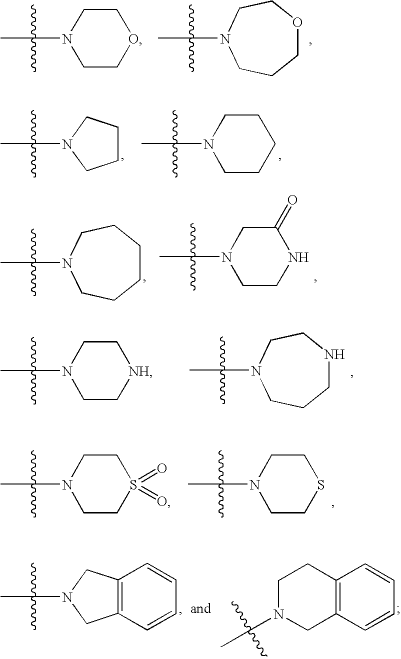
wherein said ring is optionally substituted with one or two substituents selected from the group consisting of: —CH₂OC₍₁₋₂₎alkyl, —N(C₍₁₋₄₎alkyl)₂, —OC₍₁₋₄₎alkyl, C₍₁₋₄₎alkyl, —OH, —SO₂C₍₁₋₄₎alkyl, —COC₍₁₋₄₎alkyl, —CONHC₍₁₋₄₎alkyl, phenyl, fluorophenyl, chlorophenyl, bromophenyl, and —CO₂CH₂phenyl.
R¹¹ is H, or C₍₁₋₄₎alkyl;
R¹² is C₍₁₋₄₎alkyl, indolyl, phenyl, heteroaryl, C₍₃₋₇₎cycloalkyl, morpholinyl, piperidinyl, piperazinyl, N-methyl piperazinyl, tetrahydropyranyl, pyrrolidinyl, C₍₁₋₂₎alkyl-phenyl, C₍₁₋₂₎alkyl-indolyl or C₍₁₋₂₎alkyl-heteroaryl; wherein said indolyl, phenyl, heteroaryl, C₍₁₋₂₎alkyl-phenyl, C₍₁₋₂₎alkyl-indolyl or C₍₁₋₂₎alkyl-heteroaryl are optionally substituted with one or two substituents selected from the group consisting of Cl, F, —N(C₍₁₋₄₎alkyl)₂, —OCF₃, —CF₃, —OC₍₁₋₄₎alkyl, and C₍₁₋₄₎alkyl;
   and solvates, hydrates, tautomers and pharmaceutically acceptable salts thereof.

In a more preferred embodiment of the invention:

R¹ is H, or —CH₃;

R² is H, or —CH₃;

R³ is H, or —CH₃;

R⁴ is 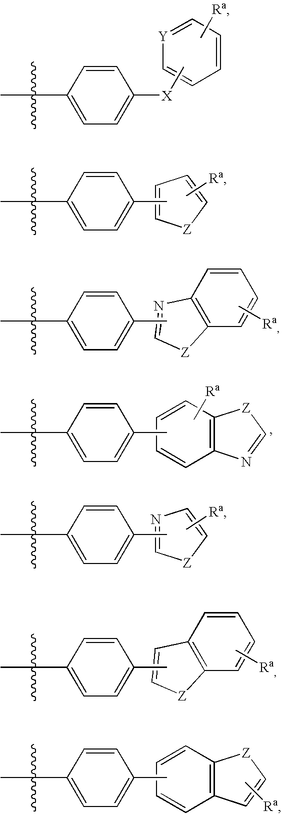 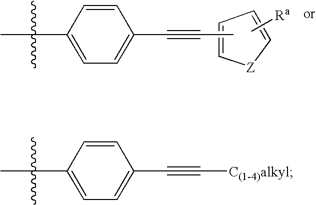
wherein:
X is O, S, 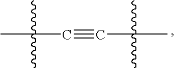
—CH═CH— (cis or trans), or a direct bond;
Y is CH or N;
Z is S or O or NH
R^{a} is one substituent selected from the group consisting of H, Cl, F, —OH, —NH₂, —N(C₍₁₋₄₎alkyl)₂, —NO₂, —CN, —CO₂H, —CONH₂, —OCF₃, —CF₃, —OC₍₁₋₄₎alkyl, and C₍₁₋₄₎alkyl;
   R⁵ is C₍₁₋₄₎alkyl, phenyl, —C₍₁₋₃₎alkyl-phenyl, —C₍₁₋₃₎alkyl-heteroaryl, —N(C₍₁₋₄₎alkyl)₂, —C₍₁₋₃₎alkyl- 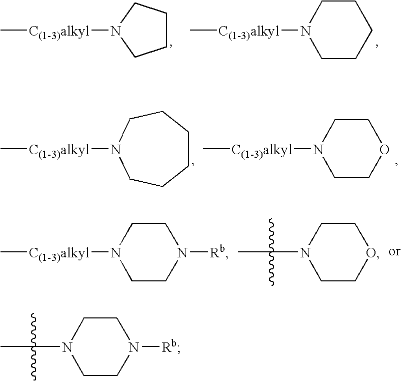
wherein said C₍₁₋₃₎alkyl-phenyl is optionally substituted with one substituent selected from the group consisting of Cl, F, —OCF₃, —CF₃, —OC₍₁₋₄₎alkyl, and C₍₁₋₄₎alkyl;
wherein R^{b} is —C₍₁₋₄₎alkyl, —SO₂C₍₁₋₄₎alkyl, —COC₍₁₋₄₎alkyl, or —CO₂CH₂-phenyl;
   R⁶ is H, C₍₁₋₄₎alkyl, allyl, —C₍₂₋₄₎alkyl-O(C₍₁₋₄₎alkyl), —C₍₁₋₃₎alkyl-phenyl, —C₍₁₋₃₎alkyl-heteroaryl, 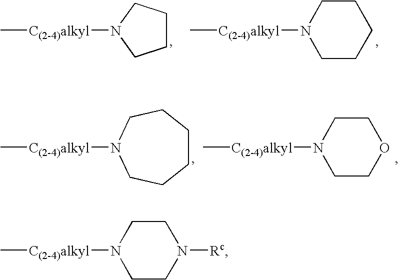
or —C₍₂₋₄₎alkyl-N(C₍₁₋₄₎alkyl)₂;
wherein said C₍₁₋₃₎alkyl-phenyl is optionally substituted with one substituent selected from the group consisting of Cl, F, —OCF₃, —CF₃, —OC₍₁₋₄₎alkyl, and C₍₁₋₄₎alkyl;
wherein R^{c} is —C₍₁₋₄₎alkyl, —SO₂C₍₁₋₄₎alkyl, —COC₍₁₋₄₎alkyl, —CO₂CH₂-phenyl;
   R⁷ is —OR⁸, —SR⁸—NR⁹R¹⁰, or —NR¹¹COR¹²;
wherein R⁸ is H, C₍₁₋₄₎alkyl, phenyl, indolyl, pyridyl, C₍₁₋₂₎alkyl-phenyl, C₍₁₋₂₎alkyl-indolyl, or C₍₁₋₂₎alkyl-pyridyl; wherein said phenyl, C₍₁₋₂₎alkyl-phenyl, C₍₁₋₂₎alkyl-indolyl, and C₍₁₋₂₎alkyl-pyridyl are optionally substituted with one or two substituents selected from the group consisting of Cl, F, —OCF₃, —CF₃, —OC₍₁₋₄₎alkyl, and C₍₁₋₄₎alkyl;
R⁹ is H, C₍₁₋₄₎alkyl, phenyl, heteroaryl, benzofused-heteroaryl, napthalenyl, C₍₃₋₇₎cycloalkyl, C₍₂₋₄₎alkyl-carbamic acid benzyl ester, —C₍₂₋₄₎alkyl-1,5,5-trimethyl-imidazolidine-2,4-dione, C₍₁₋₃₎alkyl-phenyl, C₍₁₋₃₎alkyl-heteroaryl, C₍₁₋₃₎alkyl-benzofused-heteroaryl, and wherein said C₍₁₋₃₎alkyl-phenyl, C₍₁₋₃₎alkyl-heteroaryl, C₍₁₋₃₎alkyl-benzofused-heteroaryl, phenyl, heteroaryl, and said bezo-fused heteroaryl are optionally substituted with one or two substituents selected from the group consisting of Cl, F, —OCF₃, —CF₃, —OC₍₁₋₄₎alkyl, and C₍₁₋₄₎alkyl;
R¹⁰ is H, or C₍₁₋₄₎alkyl; alternatively, R⁹ and R¹⁰ may join together with the attached nitrogen to form a ring selected from the group consisting of: 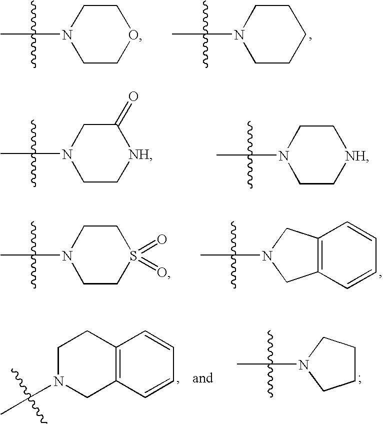
wherein said ring is optionally substituted with one or two substituents selected from the group consisting of: —CH₂OC₍₁₋₂₎alkyl, —N(C₍₁₋₃₎alkyl)₂, —OC₍₁₋₃₎alkyl, C₍₁₋₃₎alkyl, —OH, —SO₂CH₃, —COC₍₁₋₃₎alkyl, —CONHC₍₁₋₃₎alkyl, phenyl, chlorophenyl, bromophenyl, and —CO₂CH₂phenyl.
R¹¹ is H, or C₍₁₋₄₎alkyl;
R¹² is C₍₁₋₄₎alkyl, indolyl, phenyl, heteroaryl, C₍₅₋₆₎cycloalkyl, morpholinyl, piperidinyl, piperazinyl, N-methyl piperazinyl, tetrahydropyranyl, pyrrolidinyl; wherein said indolyl, phenyl, or heteroaryl, are optionally substituted with one or two substituents selected from the group consisting of Cl, F, —N(C₍₁₋₄₎alkyl)₂, —OCF₃, —CF₃, —OC₍₁₋₄₎alkyl, and C₍₁₋₄₎alkyl;
   and solvates, hydrates, tautomers and pharmaceutically acceptable salts thereof.

In a particularly preferred embodiment of the invention:

R¹ is H, or —CH₃;

R² is H, or —CH₃;

R³ is H, or —CH₃;

R⁴ is 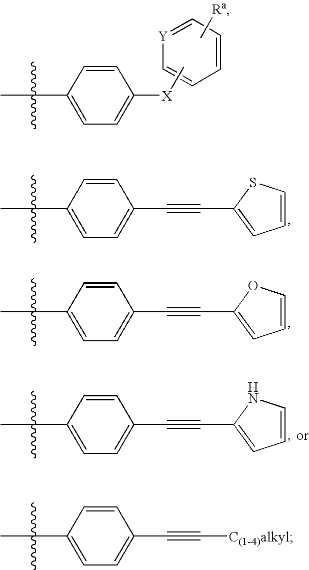
wherein:
X is O, S, 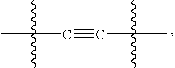
—CH═CH— (cis or trans), or a direct bond;
Y is CH or N;
R^{a} is one substituent selected from the group consisting of H, Cl, F, —OH, —NH₂, —N(C₍₁₋₄₎alkyl)₂, —NO₂, —CN, —CO₂H, —CONH₂, —OCF₃, —CF₃, —OC₍₁₋₄₎alkyl, and C₍₁₋₄₎alkyl;
   R⁵ is C₍₁₋₄₎alkyl, phenyl, —N(CH₃)₂, —C₍₁₋₃₎alkyl-phenyl, or —C₍₁₋₃₎alkyl-pyridyl;
   R⁶ is H, C₍₁₋₄₎alkyl, allyl, —C₍₁₋₃₎alkyl-phenyl, or 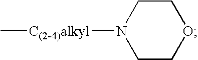
   R⁷ is —OR⁸, —SR⁸—NR⁹R¹⁰, or —NR¹¹COR¹²;
wherein R⁸ is H, C₍₁₋₄₎alkyl, phenyl, C₍₁₋₂₎alkyl-phenyl; wherein said phenyl, and C₍₁₋₂₎alkyl-phenyl, are optionally substituted with one or two substituents selected from the group consisting of Cl, F, —OCF₃, —CF₃, —OC₍₁₋₄₎alkyl, and C₍₁₋₄₎alkyl;
R⁹ is H, C₍₁₋₄₎alkyl, phenyl, fluorophenyl, difluorophenyl, fluorochlorophenyl, chlorophenyl, trifluoromethylphenyl, dimethoxyphenyl, methoxyphenyl, trifluoromethoxyphenyl, napthalenyl, cyclopentyl, indolyl, C₍₁₋₂₎alkyl-indolyl, 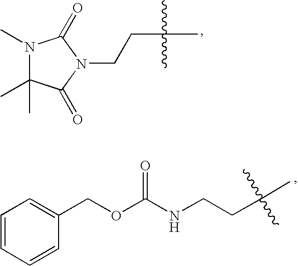
C₁₋₂alkyl-pridyl, or C₍₁₋₂₎alkyl-phenyl;
R¹⁰ is H, or C₍₁₋₄₎alkyl; alternatively, R⁹ and R¹⁰ may join together with the attached nitrogen to form a ring selected from the group consisting of: 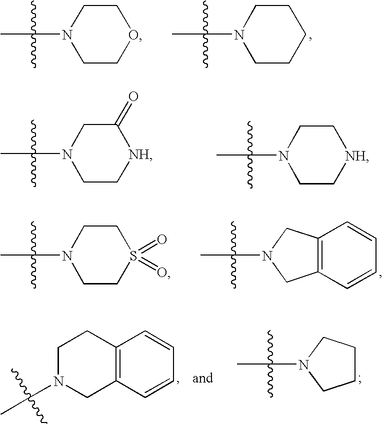
wherein said ring is optionally substituted with one or two substituents selected from the group consisting of: —CH₂OC₍₁₋₂₎alkyl, —N(C₍₁₋₃₎alkyl)₂, —OC₍₁₋₃₎alkyl, C₍₁₋₃₎alkyl, —OH, —SO₂CH₃, —COC₍₁₋₃₎alkyl, —CONHC₍₁₋₃₎alkyl, phenyl, chlorophenyl, bromophenyl, and —CO₂CH₂phenyl.
R¹¹ is H, or C₍₁₋₄₎alkyl;
R¹² is C₍₁₋₄₎alkyl, indolyl, phenyl, C₍₅₋₆₎cycloalkyl, tetrahydropyranyl; wherein said indolyl, or phenyl are optionally substituted with one substituent selected from the group consisting of Cl, F, —N(C₍₁₋₄₎alkyl)₂, —OCF₃, —CF₃, —OC₍₁₋₄₎alkyl, and C₍₁₋₄₎alkyl;
   and solvates, hydrates, tautomers and pharmaceutically acceptable salts thereof.

In an especially preferred embodiment of the invention:

R¹ is H, or CH₃;

R² is H;

R³ is H;

R⁴ is 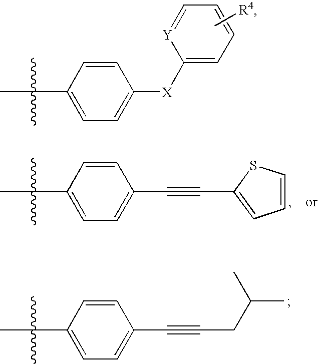
wherein:
X is O, S, 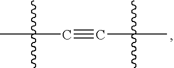
or a direct bond;
Y is CH or N;
R^{a} is H, Cl, —CF₃, —OCH₃,
   R⁵ is C₍₁₋₃₎alkyl, phenyl, —N(CH₃)₂, 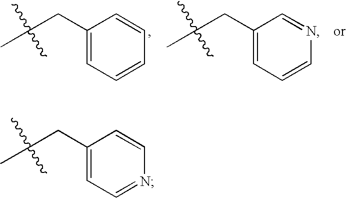
   R⁶ is H, C₍₁₋₄₎alkyl, allyl, or 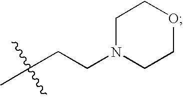
   R⁷ is —OR⁸, —NR⁹R¹⁰, or —NR¹¹COR¹²;
wherein R⁸ is H, C₍₁₋₃₎alkyl, or 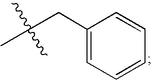
R⁹ is H, C₍₁₋₄₎alkyl, phenyl, fluorophenyl, difluorophenyl, fluorochlorophenyl, chlorophenyl, trifluoromethylphenyl, methoxyphenyl, trifluoromethoxyphenyl, napthalenyl, cyclopentyl, 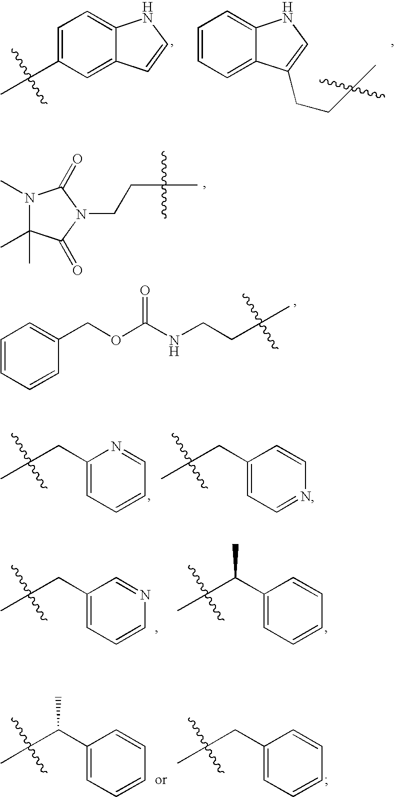
R¹⁰ is H, or C₍₁₋₃₎alkyl; alternatively, R⁹ and R¹⁰ may join together with the attached nitrogen to form a ring selected from the group consisting of: 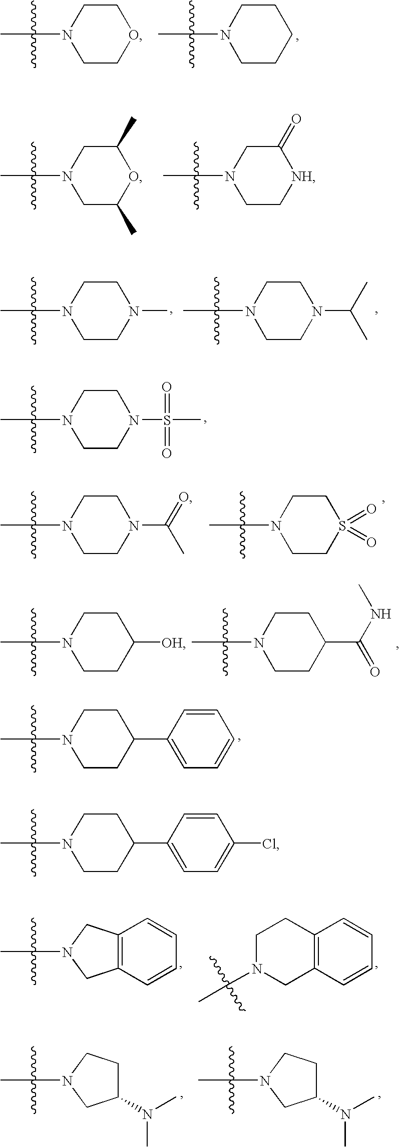 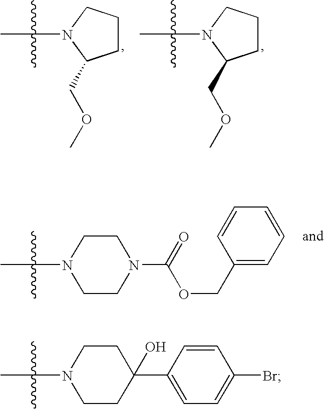
R¹¹ is H, or C₍₁₋₄₎alkyl;
R¹² is 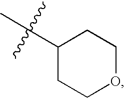
or C₍₁₋₄₎alkyl;
   and solvates, hydrates, tautomers and pharmaceutically acceptable salts thereof.

In another embodiment, the invention comprises a compound selected from Table I

TABLE I

| Cpd. | | |
|---|---|---|
| # | Structure | Name |
| 1 | 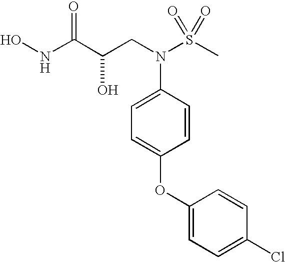 | N-Hydroxy (2S)-2-hydroxy-3-{N-[4-(4- chlorophenoxy)phenyl], N- methanesulfonylamino}propionamide |
| 2 | 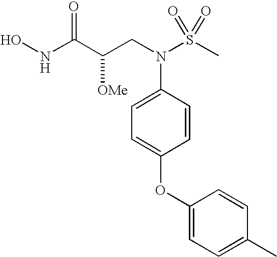 | N-Hydroxy (2S)-2-methoxy-3-{N-[4-(4- methylphenoxy)phenyl], N- methanesulfonylamino}propionamide |
| 3 | 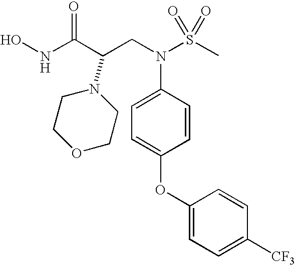 | N-Hydroxy (2S)-2-(morpholin-4-yl)-3-{N-[4- (4-trifluoromethylphenoxy)phenyl], N- methanesulfonylamino}propionamide |
| 4 | 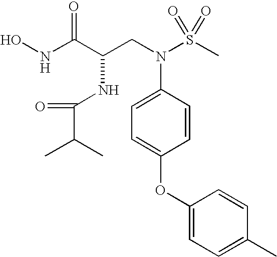 | N-Hydroxy (2S)-3-{N-[4-(4- methylphenoxy)phenyl], N- methanesulfonylamino}-2-(2- propylcarbonylamino)propionamide |
| 5 | 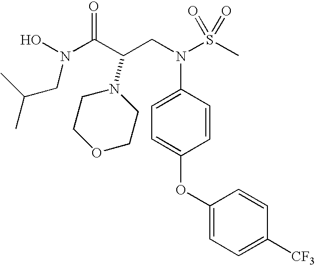 | N-Hydroxy, N-(2-methylpropyl) (2S)-2- (morpholin-4-yl)-3-{N-[4-(4- trifluoromethylphenoxy)phenyl], N- methanesulfonylamino}propionamide |
| 6 | 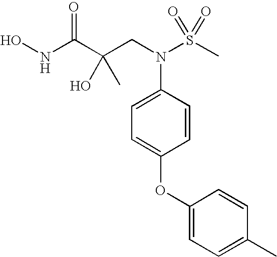 | N-Hydroxy 2-hydroxy-2-methyl-3-{N-[4-(4- methylphenoxy)phenyl], N- methanesulfonylamino}propionamide |
| 7 | 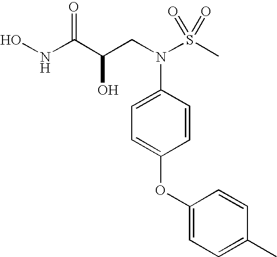 | N-Hydroxy (2R)-2-hydroxy-3-{N-[4-(4- methylphenoxy)phenyl], N- methanesulfonylamino}propionamide |
| 8 | 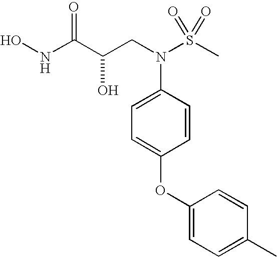 | N-Hydroxy (2S)-2-hydroxy-3-{N-[4-(4- methylphenoxy)phenyl], N- methanesulfonylamino}propionamide |
| 9 | 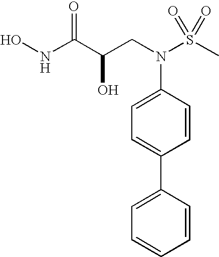 | N-Hydroxy (2R)-2-hydroxy-3-[N-(4- phenyl)phenyl, N- methanesulfonylamino]propionamide |
| 10 | 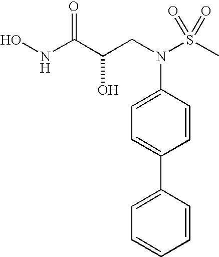 | N-Hydroxy (2S)-2-hydroxy-3-[N-(4- phenyl)phenyl, N- methanesulfonylamino]propionamide |
| 11 | 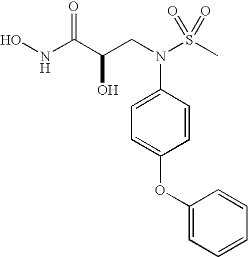 | N-Hydroxy (2R)-2-hydroxy-3-[N-(4- phenoxy)phenyl, N- methanesulfonylamino]propionamide |
| 12 | 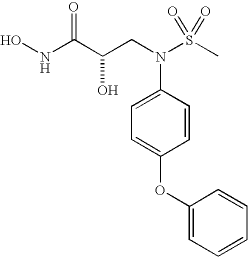 | N-Hydroxy (2S)-2-hydroxy-3-[N-(4- phenoxy)phenyl, N- methanesulfonylamino]propionamide |
| 13 | 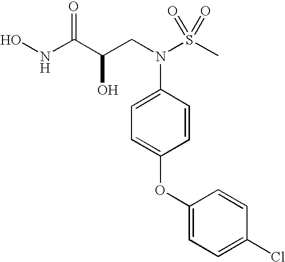 | N-Hydroxy (2R)-2-hydroxy-3-{N-[4-(4- chlorophenoxy)phenyl], N- methanesulfonylamino}propionamide |
| 14 | 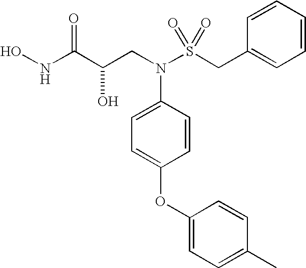 | N-Hydroxy (2S)-2-hydroxy-3-{N-[4-(4- methylphenoxy)phenyl], N- phenylmethylsulfonylamino}propionamide |
| 15 | 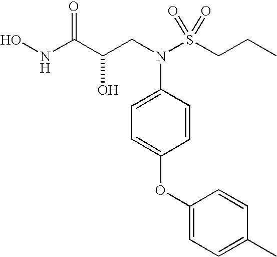 | N-Hydroxy (2S)-2-hydroxy-3-{N-[4-(4- methylphenoxy)phenyl], N-(1- propyl)sulfonylamino}propionamide |
| 16 | 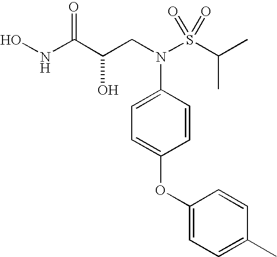 | N-Hydroxy (2S)-2-hydroxy-3-{N-[4-(4- methylphenoxy)phenyl], N-(2- propyl)sulfonylamino}propionamide |
| 17 | 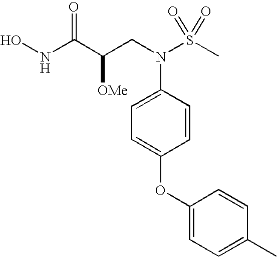 | N-Hydroxy (2R)-2-methoxy-3-{N-[4-(4- methylphenoxy)phenyl], N- methanesulfonylamino}propionamide |
| 18 | 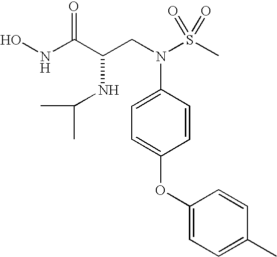 | N-Hydroxy (2S)-3-{N-[4-(4- methylphenoxy)phenyl], N- methanesulfonylamino}-2-[(2- propyl)amino]propionamide |
| 19 | 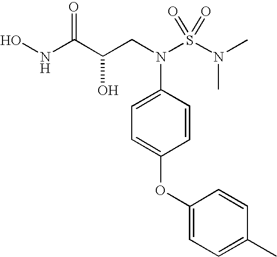 | N-Hydroxy (2S)-2-hydroxy-3-{N-[4-(4- methylphenoxy)phenyl], N- (dimethylamino)sulfonylamino}propionamide |
| 20 | 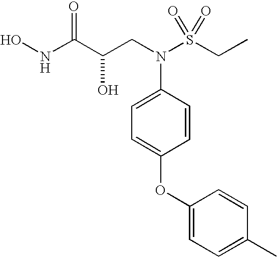 | N-Hydroxy (2S)-2-hydroxy-3-{N-[4-(4- methylphenoxy)phenyl], N- ethylsulfonylamino}propionamide |
| 21 | 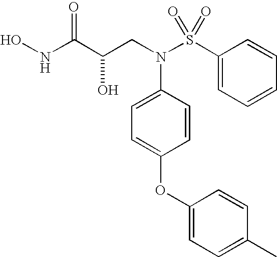 | N-Hydroxy (2S)-2-hydroxy-3-{N-[4-(4- methylphenoxy)phenyl], N- phenylsulfonylamino}propionamide |
| 22 | 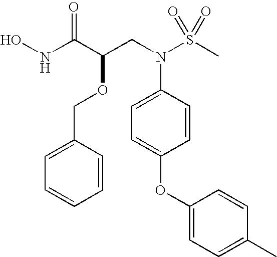 | N-Hydroxy (2R)-2-benzyloxy-3-{N-[4-(4- methylphenoxy)phenyl], N- methanesulfonylamino}propionamide |
| 23 | 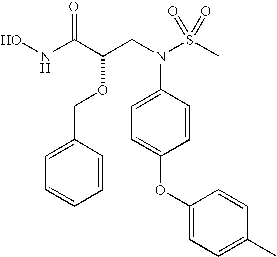 | N-Hydroxy (2S)-2-benzyloxy-3-{N-[4-(4- methylphenoxy)phenyl], N- methanesulfonylamino}propionamide |
| 24 | 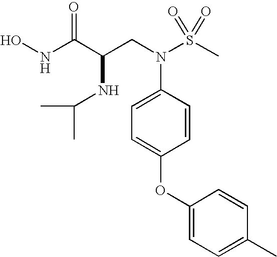 | N-Hydroxy (2R)-3-{N-[4-(4- methylphenoxy)phenyl], N- methanesulfonylamino}-2-(2- propyl)aminopropionamide |
| 25 | 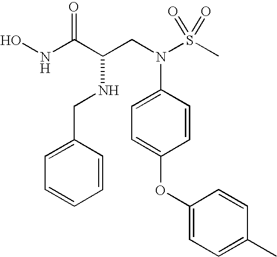 | N-Hydroxy (2S)-2-benzylamino-3-{N-[4-(4- methylphenoxy)phenyl], N- methanesulfonylamino}propionamide |
| 26 | 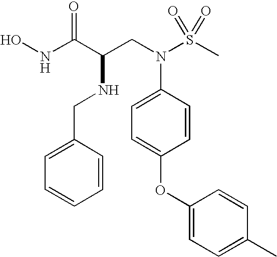 | N-Hydroxy (2R)-2-benzylamino-3-{N-[4-(4- methylphenoxy)phenyl], N- methanesulfonylamino}propionamide |
| 27 | 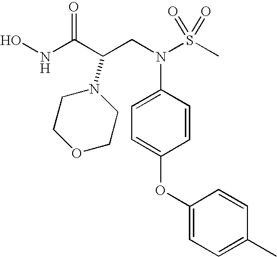 | N-Hydroxy (2S)-3-{N-[4-(4- methylphenoxy)phenyl], N- methanesulfonylamino}-2-(morpholin-4- yl)propionamide |
| 28 | 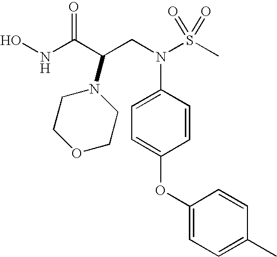 | N-Hydroxy (2R)-3-{N-[4-(4- methylphenoxy)phenyl], N- methanesulfonylamino}-2-(morpholin-4- yl)propionamide |
| 29 | 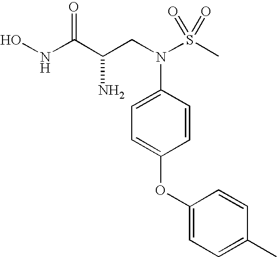 | N-Hydroxy (2S)-2-amino-3-{N-[4-(4- methylphenoxy)phenyl], N- methanesulfonylamino}propionamide |
| 30 | 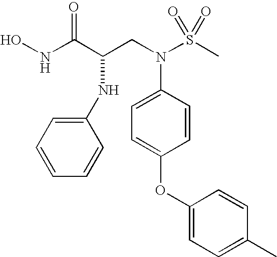 | N-Hydroxy (2S)-3-{N-[4-(4- methylphenoxy)phenyl], N- methanesulfonylamino}-2- phenylaminopropionamide |
| 31 | 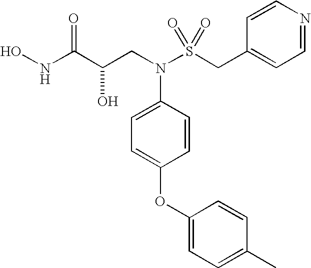 | N-Hydroxy (2S)-2-hydroxy-3-{N-[4-(4- methylphenoxy)phenyl], N-(pryidin-4- yl)methylsulfonylamino}propionamide |
| 32 | 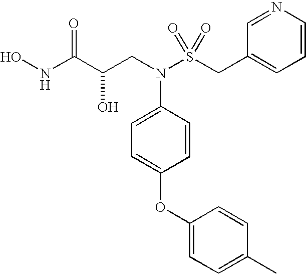 | N-Hydroxy (2S)-2-hydroxy-3-{N-[4-(4- methylphenoxy)phenyl], N-(pryidin-3- yl)methylsulfonylamino}propionamide |
| 33 | 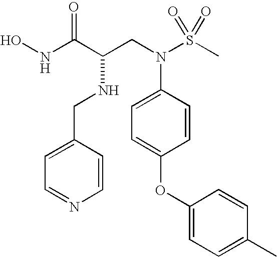 | N-Hydroxy (2S)-3-{N-[4-(4- methylphenoxy)phenyl], N- methanesulfonylamino}-2-(pyridine-4- yl)methylaminopropionamide |
| 34 | 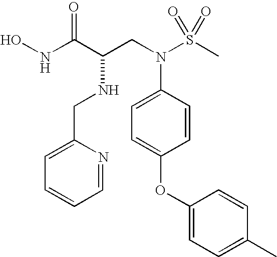 | N-Hydroxy (2S)-3-{N-[4-(4- methylphenoxy)phenyl], N- methanesulfonylamino}-2-(pyridin2- yl)methylaminopropionamide |
| 35 | 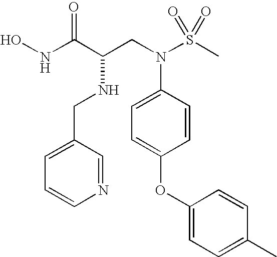 | N-Hydroxy (2S)-3-{N-[4-(4- methylphenoxy)phenyl], N- methanesulfonylamino}-2-(pyridine-3- yl)methylaminopropionamide |
| 36 | 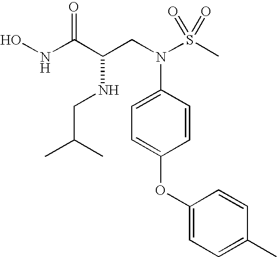 | N-Hydroxy (2S)-3-{N-[4-(4- methylphenoxy)phenyl], N- methanesulfonylamino}-2-(2- methylpropyl)aminopropionamide |
| 37 | 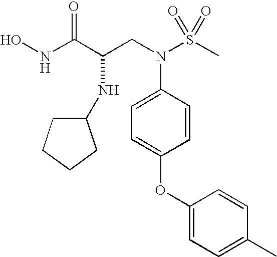 | N-Hydroxy (2S)-2-cyclopentylamino-3-{N-[4- (4-methylphenoxy)phenyl], N- methanesulfonylamino}propionamide |
| 38 | 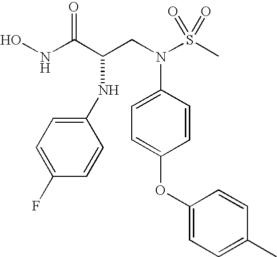 | N-Hydroxy (2S)-2-(4-fluorophenyl)amino-3- {N-[4-(4-methylphenoxy)phenyl], N- methanesulfonylamino}propionamide |
| 39 | 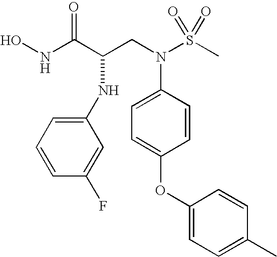 | N-Hydroxy (2S)-2-(3-fluorophenyl)amino-3- {N-[4-(4-methylphenoxy)phenyl], N- methanesulfonylamino}propionamide |
| 40 | 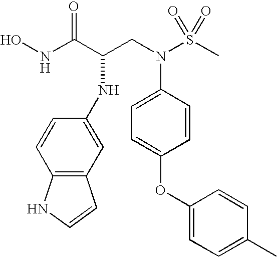 | N-Hydroxy (2S)-2-(indol-5-yl)amino-3-{N-[4- (4-methylphenoxy)phenyl], N- methanesulfonylamino}propionamide |
| 41 | 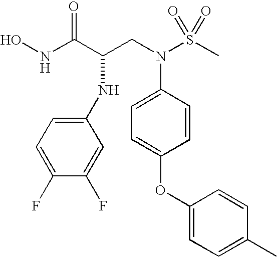 | N-Hydroxy (2S)-2-(3,4-difluorophenyl)amino- 3-{N-[4-(4-methylphenoxy)phenyl], N- methanesulfonylamino}propionamide |
| 42 | 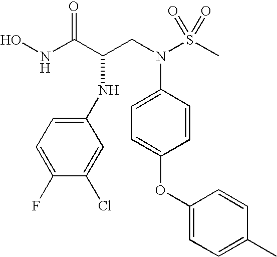 | N-Hydroxy (2S)-2-(3-chloro-4- fluorophenyl)amino-3-{N-[4-(4- methylphenoxy)phenyl], N- methanesulfonylamino}propionamide |
| 43 | 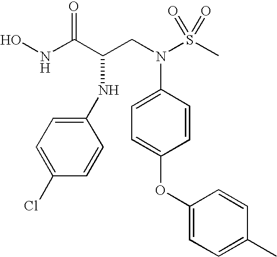 | N-Hydroxy (2S)-2-(4-chlorophenyl)amino-3- {N-[4-(4-methylphenoxy)phenyl], N- methanesulfonylamino}propionamide |
| 44 | 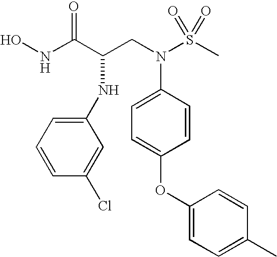 | N-Hydroxy (2S)-2-(3-chlorophenyl)amino-3- {N-[4-(4-methylphenoxy)phenyl], N- methanesulfonylamino}propionamide |
| 45 | 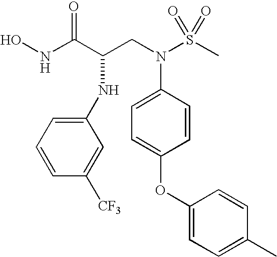 | N-Hydroxy (2S)-3-{N-[4-(4- methylphenoxy)phenyl], N- methanesulfonylamino}-2-(3- trifluorormethylphenyl)aminopropionamide |
| 46 | 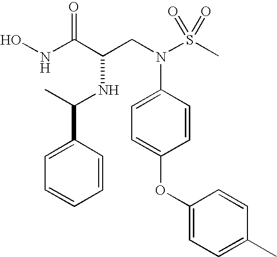 | N-Hydroxy (2S)-3-{N-[4-(4- methylphenoxy)phenyl], N- methanesulfonylamino}-2-[(1R)-1-phenyl-1- ethyl]aminopropionamide |
| 47 | 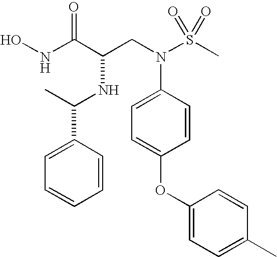 | N-Hydroxy (2S)-3-{N-[4-(4- methylphenoxy)phenyl], N- methanesulfonylamino}-2-[(1S)-1-phenyl-1- ethyl]aminopropionamide |
| 48 | 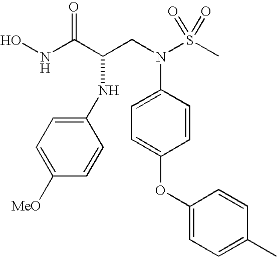 | N-Hydroxy (2S)-2-(4-methoxyphenyl)amino- 3-{N-[4-(4-methylphenoxy)phenyl], N- methanesulfonylamino}propionamide |
| 49 | 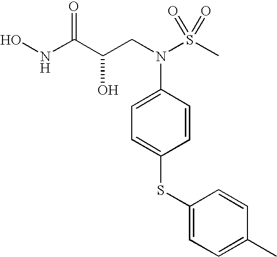 | N-Hydroxy (2S)-2-hydroxy-3-{N-[4-(4- methylthiophenoxy)phenyl], N- methanesulfonylamino}propionamide |
| 50 | 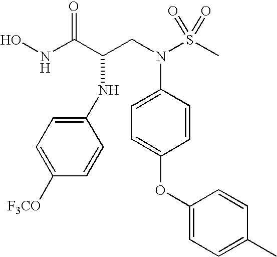 | N-Hydroxy (2S)-3-{N-[4-(4- methylphenoxy)phenyl], N- methanesulfonylamino}-2-(4- trifluoromethoxyphenyl)aminopropionamide |
| 51 | 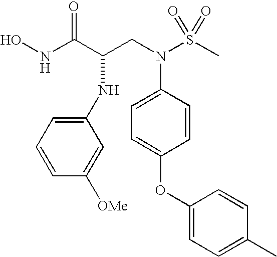 | N-Hydroxy (2S)-2-(3-methoxyphenyl)amino- 3-{N-[4-(4-methylphenoxy)phenyl], N- methanesulfonylamino}propionamide |
| 52 | 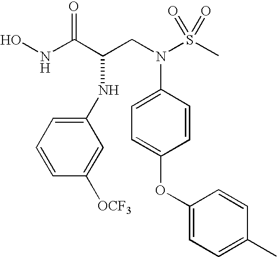 | N-Hydroxy (2S)-3-{N-[4-(4- methylphenoxy)phenyl], N- methanesulfonylamino}-2-(3- trifluoromethoxyphenyl)aminopropionamide |
| 53 | 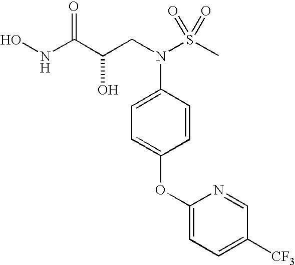 | N-Hydroxy (2S)-2-hydroxy-3-{N-{4-[(5- trifluoromethylpyridin-2-yl)oxy]phenyl}, N- methanesulfonylamino}propionamide |
| 54 | 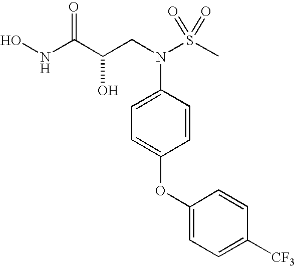 | N-Hydroxy (2S)-2-hydroxy-3-{N-[4-(4- trifluoromethylphenoxy)phenyl], N- methanesulfonylamino}propionamide |
| 55 | 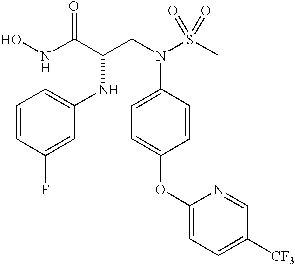 | N-Hydroxy (2S)-2-(3-fluorophenyl)amino-3- {N-{4-[(5-trifluoromethylpyridin-2- yl)oxy]phenyl}, N- methanesulfonylamino}propionamide |
| 56 | 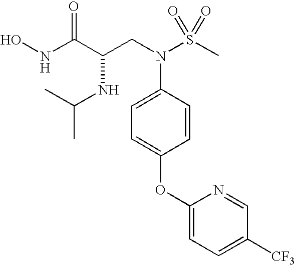 | N-Hydroxy (2S)-2-(2-propyl)amino-3-{N-{4- [(5-trifluoromethylpyridin-2-yl)oxy]phenyl}, N- methanesulfonylamino}propionamide |
| 57 | 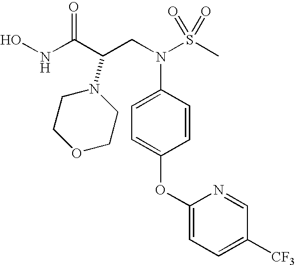 | N-Hydroxy (2S)-2-(morpholin-4-yl)-3-{N-{4- [(5-trifluoromethylpyridin-2-yl)oxy]phenyl}, N- methanesulfonylamino}propionamide |
| 58 | 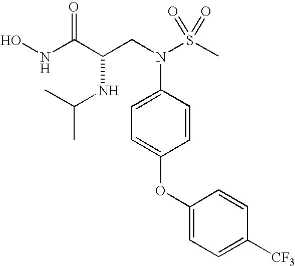 | N-Hydroxy (2S)-2-(2-propyl)amino-3-{N-[4- (4-trifluoromethylphenoxy)phenyl], N- methanesulfonylamino}propionamide |
| 59 | 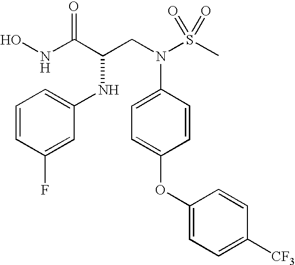 | N-Hydroxy (2S)-2-(3-fluorophenyl)amino-3- N{N-[4-(4-trifluoromethylphenoxy)phenyl], N- methanesulfonylamino}propionamide |
| 60 | 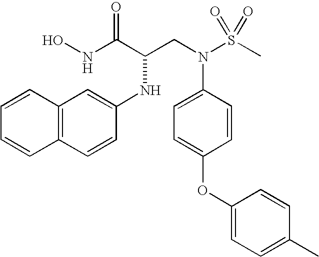 | N-Hydroxy (2S)-3-{N-[4-(4- methylphenoxy)phenyl], N- methanesulfonylamino}-2-(naphthylen-2- yl)aminopropionamide |
| 61 | 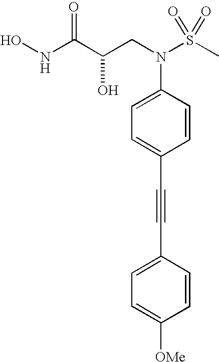 | N-Hydroxy (2S)-2-hydroxy-3-{N-[4-(4- methoxyphenylacetylene)phenyl], N- methanesulfonylamino}propionamide |
| 62 | 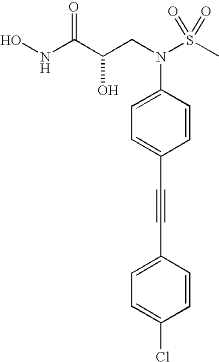 | N-Hydroxy (2S)-2-hydroxy-3-{N-[4-(4- chlorophenylacetylene)phenyl], N- methanesulfonylamino}propionamide |
| 63 | 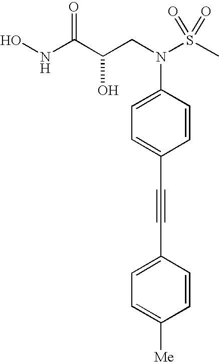 | N-Hydroxy (2S)-2-hydroxy-3-{N-[4-(4- methylphenylacetylene)phenyl], N- methanesulfonylamino}propionamide |
| 64 | 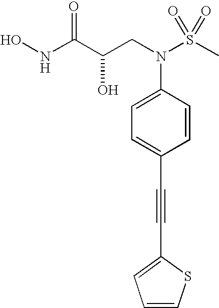 | N-Hydroxy (2S)-2-hydroxy-3-{N-[4-(4- thiophen-2-ylacetylene)phenyl], N- methanesulfonylamino}propionamide |
| 65 | 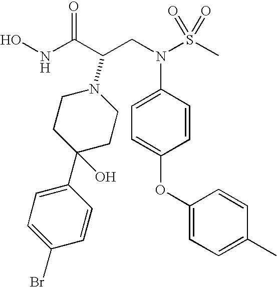 | N-Hydroxy (2S)-2-[4-(4-bromophenyl)-4- hydroxypiperidin-1-yl]-3-{N-[4-(4- methylphenoxy)phenyl], N- methanesulfonylamino}propionamide |
| 66 | 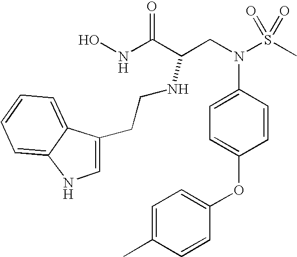 | N-Hydroxy (2S)-2-[(2-(indol-3-yl)ethylamino]- 3-{N-[4-(4-methylphenoxy)phenyl], N- methanesulfonylamino}propionamide |
| 67 | 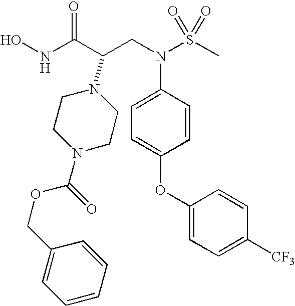 | N-Hydroxy (2S)-2-[(4- benzyloxycarbonyl)piperazin-1-yl]-3-{N-[4-(4- trifluoromethylphenoxy)phenyl], N- methanesulfonylamino}propionamide |
| 68 | 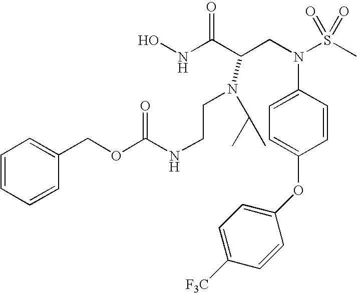 | N-Hydroxy (2S)-2-{N-(2-propyl), N-[2- benzyloxycarbonylamino)ethyl]amino}-3-{N- [4-(4-trifluoromethylphenoxy)phenyl], N- methanesulfonylamino}propionamide |
| 69 | 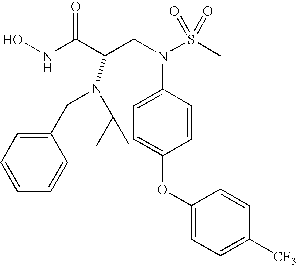 | N-Hydroxy (2S)-2-[N-benzyl-N-(2- propyl)amino]-3-{N-[4-(4- trifluoromethylphenoxy)phenyl], N- methanesulfonylamino}propionamide |
| 70 | 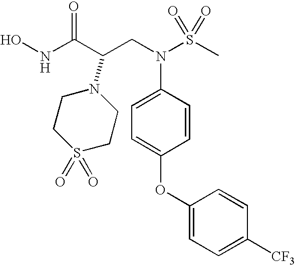 | N-Hydroxy (2S)-2-[1,1-bis(oxo)thiomorpholin- 4-yl]-3-{N-[4-(4- trifluoromethylphenoxy)phenyl], N- methanesulfonylamino}propionamide |
| 71 | 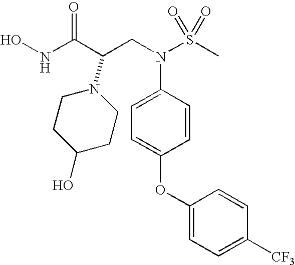 | N-Hydroxy (2S)-2-(4-hydroxypiperidin-1-yl)- 3-{N-[4-(4-trifluoromethylphenoxy)phenyl], N- methanesulfonylamino}propionamide |
| 72 | 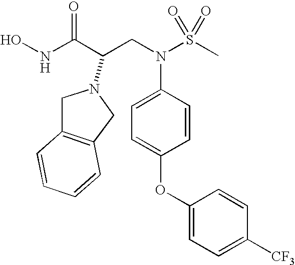 | N-Hydroxy (2S)-2-(isoindolin-2-yl)-3-{N-[4-(4- trifluoromethylphenoxy)phenyl], N- methanesulfonylamino}propionamide |
| 73 | 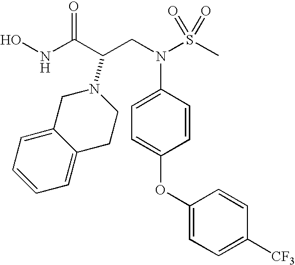 | N-Hydroxy (2S)-2-(1,2,3,4- tetrahydroisoquinolin-2-yl)-3-{N-[4-(4- trifluoromethylphenoxy)phenyl], N- methanesulfonylamino}propionamide |
| 74 | 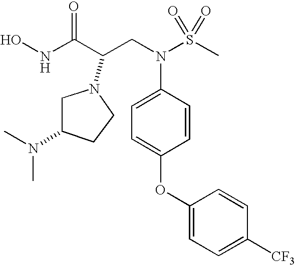 | N-Hydroxy (2S)-2-[(3S)-3-N,N- dimethylaminopyrrolidin-1-yl]-3-{N-[4-(4- trifluoromethylphenoxy)phenyl], N- methanesulfonylamino}propionamide |
| 75 | 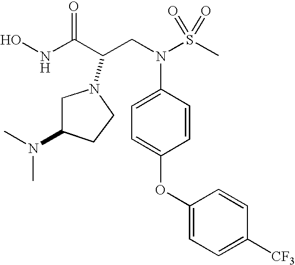 | N-Hydroxy (2S)-2-[(3R)-3-N,N- dimethylaminopyrrolidin-1-yl]-3-{N-[4-(4- trifluoromethylphenoxy)phenyl], N- methanesulfonylamino}propionamide |
| 76 | 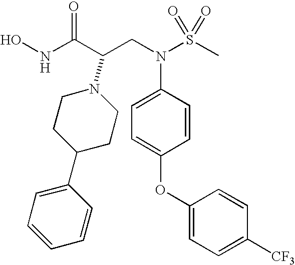 | N-Hydroxy (2S)-3-{N-[4-(4- trifluoromethylphenoxy)phenyl], N- methanesulfonylamino}-2-(4-phenylpiperidin- 1-yl)propionamide |
| 77 | 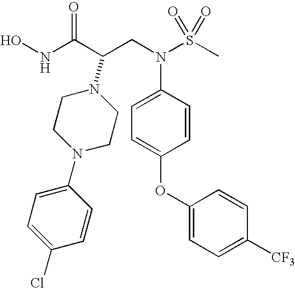 | N-Hydroxy (2S)-2-[4-(4- chlorophenyl)piperazin-1-yl]-3-{N-[4-(4- trifluoromethylphenoxy)phenyl], N- methanesulfonylamino}propionamide |
| 78 | 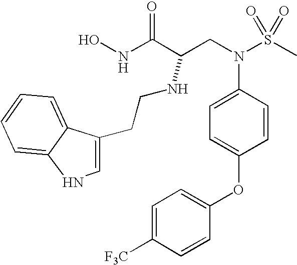 | N-Hydroxy (2S)-2-[(2-(indol-3-yl)ethylamino]- 3-{N-[4-(4-trifluoromethylphenoxy)phenyl], N- methanesulfonylamino}propionamide |
| 79 | 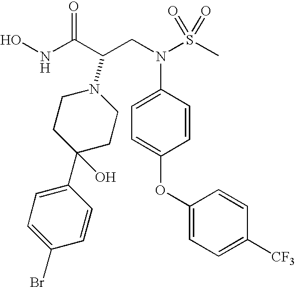 | N-Hydroxy (2S)-2-[4-(4-bromophenyl)-4- hydroxypiperidin-1-yl]-3-{N-[4-(4- trifluoromethylphenoxy)phenyl], N- methanesulfonylamino}propionamide |
| 80 | 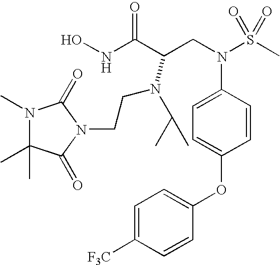 | N-Hydroxy (2S)-2-{N-(2-propyl), N-[2-(1,5,5- trimethylhydantoin-3-yl)ethyl]amino}-3-{N-[4- (4-trifluoromethylphenoxy)phenyl]-N- methanesulfonylamino}propionamide |
| 81 | 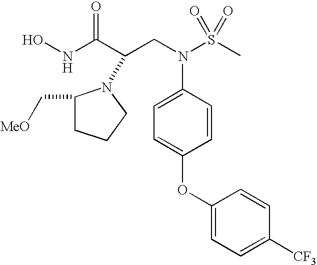 | N-Hydroxy (2S)-2-[(2R)-2- methoxymethylpyrrolidin-1-yl]-3-{N-[4-(4- trifluoromethylphenoxy)phenyl], N- methanesulfonylamino}propionamide |
| 82 | 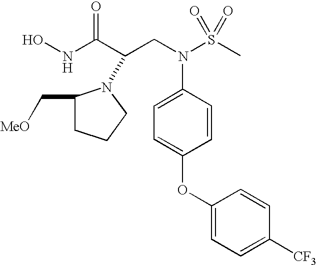 | N-Hydroxy (2S)-2-[(2S)-2- methoxymethylpyrrolidin-1-yl]-3-{N-[4-(4- trifluoromethylphenoxy)phenyl], N- methanesulfonylamino}propionamide |
| 83 | 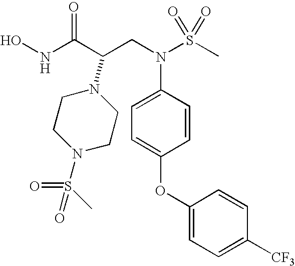 | N-Hydroxy (2S)-2-(4- methanesulfonylpiperazin-1-yl)-3-{N-[4-(4- trifluoromethylphenoxy)phenyl], N- methanesulfonylamino}propionamide |
| 84 | 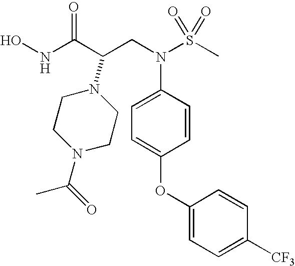 | N-Hydroxy (2S)-2-(4-acetylpiperazin-1-yl)-3- {N-[4-(4-trifluoromethylphenoxy)phenyl], N- methanesulfonylamino}propionamide |
| 85 | 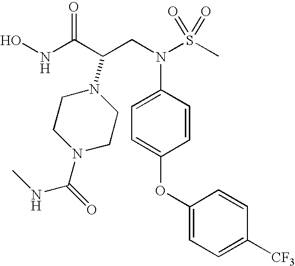 | N-Hydroxy (2S)-2-[4-(N- methylaminocarbonyl)piperidin-1-yl]-3-{N-[4- (4-trifluoromethylphenoxy)phenyl], N- methanesulfonylamino}propionamide |
| 86 | 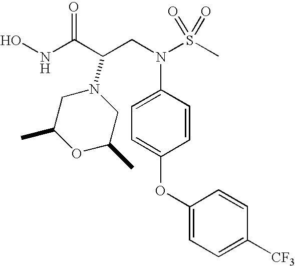 | N-Hydroxy (2S)-2-(cis-2,6- dimethylmorpholin-4-yl)-3-{N-[4-(4- trifluoromethylphenoxy)phenyl], N- methanesulfonylamino}propionamide |
| 87 | 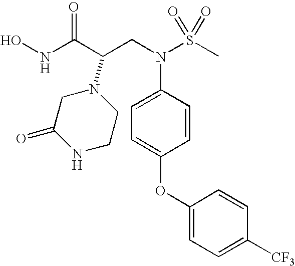 | N-Hydroxy (2S)-2-(3-oxopiperazin-1-yl)-3-{N- [4-(4-trifluoromethylphenoxy)phenyl], N- methanesulfonylamino}propionamide |
| 88 | 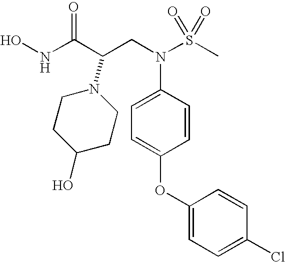 | N-Hydroxy (2S)-3-{N-[4-(4- chlorophenoxy)phenyl], N- methanesulfonylamino}-2-(4- hydroxypiperidin-1-yl)propionamide |
| 89 | 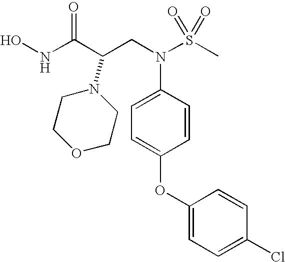 | N-Hydroxy (2S)-2-(morpholin-4-yl)-3-{N-[4- (4-chlorophenoxy)phenyl], N- methanesulfonylamino}propionamide |
| 90 | 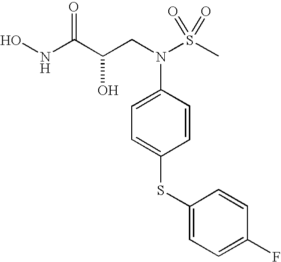 | N-Hydroxy (2S)-2-hydroxy-3-{N-[4-(4- fluorothiophenoxy)phenyl], N- methanesulfonylamino}propionamide |
| 91 | 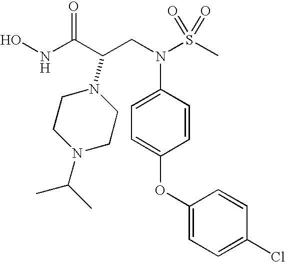 | N-Hydroxy (2S)-3-{N-[4-(4- chlorophenoxy)phenyl], N- methanesulfonylamino}-2-[4-(2- propyl)piperazin-1-yl]propionamide |
| 92 | 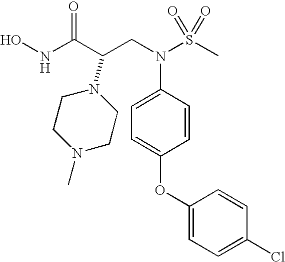 | N-Hydroxy (2S)-3-{N-[4-(4- chlorophenoxy)phenyl], N- methanesulfonylamino}-2-(4- methylpiperazin-1-yl)propionamide |
| 93 | 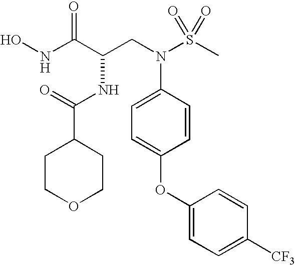 | N-Hydroxy (2S)-2-[(tetrahydropyran-4- yl)carbonylamino]-3-{N-[4-(4- trifluoromethylphenoxy)phenyl], N- methanesulfonylamino}propionamide |
| 94 | 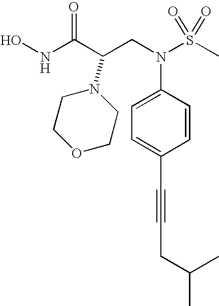 | N-Hydroxy (2S)-3-{N-[4-(4-methylpentyn-1- yl)phenyl], N-methanesulfonylamino}-2- (morpholin-4-yl)propionamide |
| 95 | 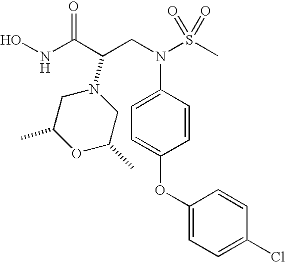 | N-Hydroxy (2S)-3-{N-[4-(4- chlorophenoxy)phenyl], N- methanesulfonylamino}-2-(cis-2,6- dimethylmorpholin-4-yl)propionamide |
| 96 | 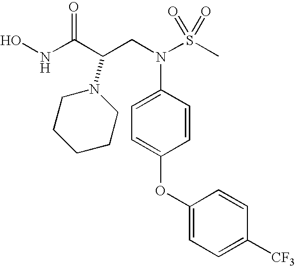 | N-Hydroxy (2S)-2-(piperidin-1-yl)-3-{N-[4-(4- trifluoromethylphenoxy)phenyl], N- methanesulfonylamino}propionamide |
| 97 | 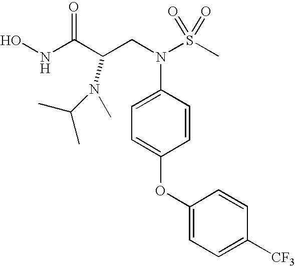 | N-Hydroxy (2S)-2-[N-methyl, N-(2- propyl)amino]-3-{N-[4-(4- trifluoromethylphenoxy)phenyl], N- methanesulfonylamino}propionamide |
| 98 | 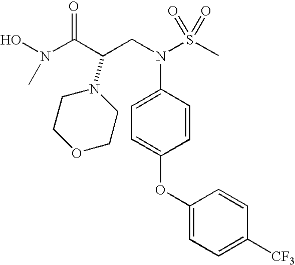 | N-Hydroxy, N-methyl (2S)-2-(morpholin-4-yl)- 3-{N-[4-(4-trifluoromethylphenoxy)phenyl], N- methanesulfonylamino}propionamide |
| 99 | 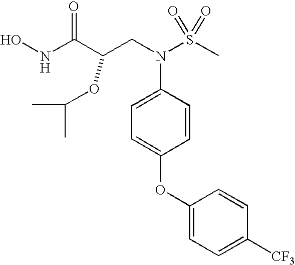 | N-Hydroxy (2S)-2-(2-propoxy)-3-{N-[4-(4- trifluoromethylphenoxy)phenyl], N- methanesulfonylamino}propionamide |
| 100 | 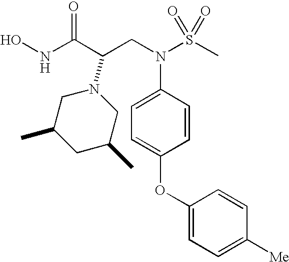 | N-Hydroxy (2S)-2-(cis-2,6- dimethylmorpholin-4-yl)-3-{N-[4-(4- methylphenoxy)phenyl], N- methanesulfonylamino}propionamide |
| 101 | 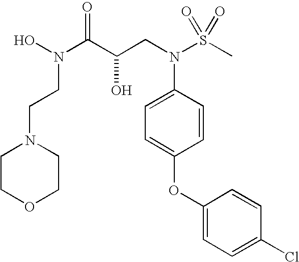 | N-Hydroxy, N-[2-(morpholin-4-yl)ethyl] (2S)-2- hydroxy-3-{N-[4-(4-chlorophenoxy)phenyl], N- methanesulfonylamino}propionamide |
| 102 | 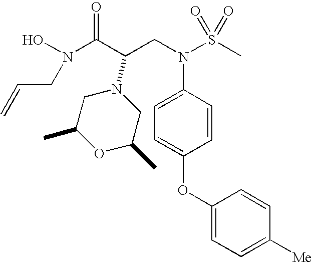 | N-(3-Allyl), N-hydroxy (2S)-2-(cis-2,6- dimethylmorpholin-4-yl)-3-{N-[4-(4- methylphenoxy)phenyl], N- methanesulfonylamino}propionamide |

and solvates, hydrates, tautomers and pharmaceutically acceptable salts thereof.

Another embodiment of the invention is a compound which is selected from the group consisting of 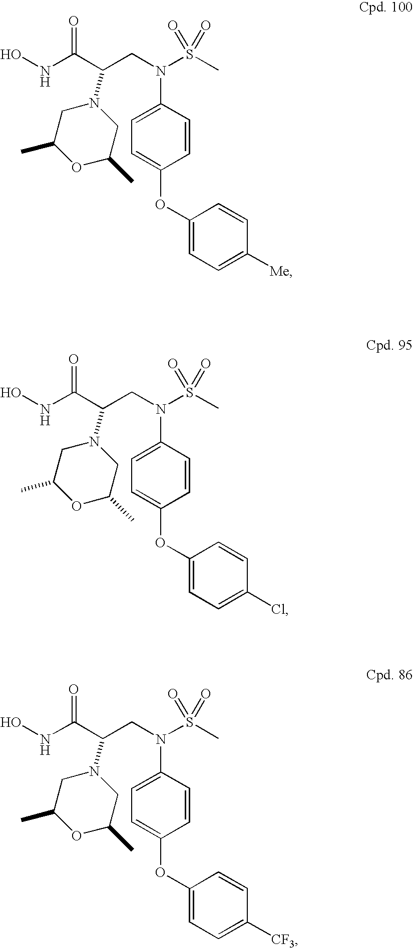 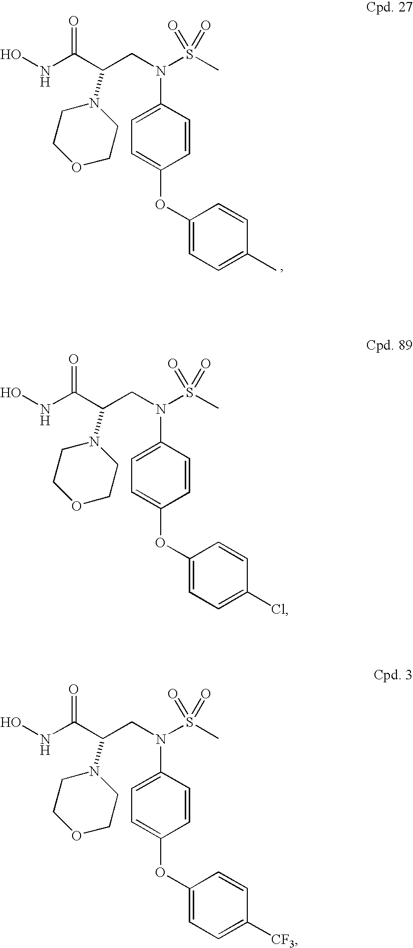 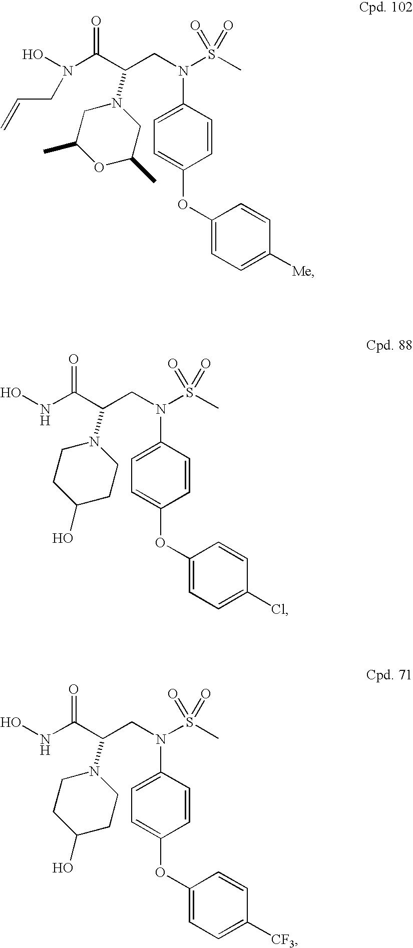 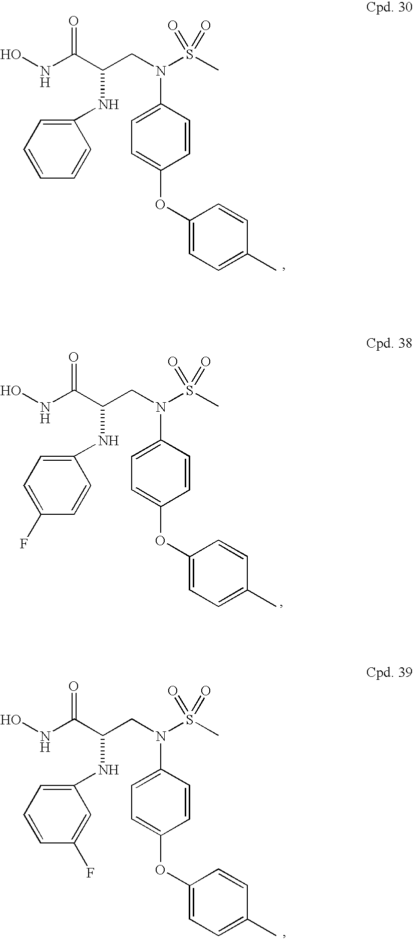
and solvates, hydrates, tautomers and pharmaceutically acceptable salts thereof.

Another embodiment of the invention is a compound which is 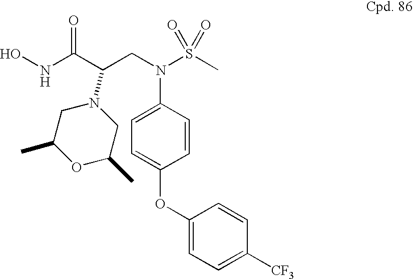
and solvates, hydrates, tautomers and pharmaceutically acceptable salts thereof.

Another embodiment of the invention is a compound which is 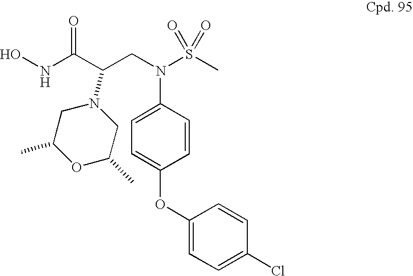
and solvates, hydrates, tautomers and pharmaceutically acceptable salts thereof.

Another embodiment of the invention is a compound which is 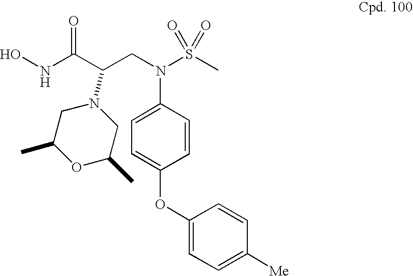
and solvates, hydrates, tautomers and pharmaceutically acceptable salts thereof.

It is recognized by those skilled in the art that a methylene group may not be stably substituted with two heteroatoms, and any compound of Formula I bearing such a methylene group is not considered part of the invention. Examples of such substitutions include but are not limited to 1-methoxy pyrrolyl, 3-hydroxy morpholinyl, and 2-hydroxy piperazinyl.

### DEFINITIONS

As used herein, the following terms are intended to have the following meanings (additional definitions are provided where needed throughout the Specification):

The term “C_{a-b}” (where a and b are integers referring to a designated number of carbon atoms) refers to an alkyl, alkoxy or cycloalkyl radical or to the alkyl portion of a radical in which alkyl appears as the prefix root containing from a to b carbon atoms inclusive. For example, C₁₋₄ denotes a radical containing 1, 2, 3 or 4 carbon atoms.

The term “alkyl,” whether used alone or as part of a substituent group, refers to a saturated branched or straight chain monovalent hydrocarbon radical, wherein the radical is derived by the removal of one hydrogen atom from a single carbon atom. Unless specifically indicated (e.g. by the use of a limiting term such as “terminal carbon atom”), substituent variables may be placed on any carbon chain atom. Typical alkyl radicals include, but are not limited to, methyl, ethyl, propyl, isopropyl and the like. Examples include C₁₋₈alkyl, C₁₋₆alkyl and C₁₋₄alkyl groups.

The term “aromatic” refers to a cyclic hydrocarbon ring system having an unsaturated, conjugated 4n+2π electron system, wherein n is an integer selected from 0, 1, or 2.

The term “aryl” refers to an aromatic cyclic hydrocarbon ring radical derived by the removal of one hydrogen atom from a single carbon atom of the ring system. Typical aryl radicals include phenyl, naphthalenyl, fluorenyl, indenyl, azulenyl, anthracenyl and the like.

The term “contacting” as used herein, refers to the addition of compound to cells such that compound is taken up by the cell.

The term “cycloalkyl” refers to a saturated or partially unsaturated monocyclic or bicyclic hydrocarbon ring radical derived by the removal of one hydrogen atom from a single ring carbon atom. Typical cycloalkyl radicals include cyclopropyl, cyclobutyl, cyclopentyl, cyclopentenyl, cyclohexyl, cyclohexenyl, cycloheptyl and cyclooctyl. Additional examples include C₃₋₈cycloalkyl, C₅₋₈cycloalkyl, C₃₋₁₂cycloalkyl, C₃₋₂₀cycloalkyl, decahydronaphthalenyl, and 2,3,4,5,6,7-hexahydro-1H-indenyl.

The term “hetero” used as a prefix for a ring system refers to the replacement of at least one ring carbon atom with one or more atoms independently selected from N, S, O or P. Examples include rings wherein 1, 2, 3 or 4 ring members are a nitrogen atom; or, 0, 1, 2 or 3 ring members are nitrogen atoms and 1 member is an oxygen or sulfur atom.

The term “heteroaryl” refers to a radical derived by the removal of one hydrogen atom from a ring carbon atom of a heteroaromatic ring system. Typical heteroaryl radicals include furyl, thienyl, pyrrolyl, oxazolyl, thiazolyl, imidazolyl, pyrazolyl, isoxazolyl, isothiazolyl, oxadiazolyl, triazolyl, thiadiazolyl, pyridinyl, pyridazinyl, pyrimidinyl, pyrazinyl, indolizinyl, indolyl, isoindolyl, benzo[b]furyl, benzo[b]thienyl, indazolyl, benzimidazolyl, benzthiazolyl, purinyl, 4H-quinolizinyl, quinolinyl, isoquinolinyl, cinnolinyl, phthalzinyl, quinazolinyl, quinoxalinyl, 1,8-naphthyridinyl, pteridinyl and the like.

The term “heterocyclyl” refers to a saturated or partially unsaturated monocyclic ring radical derived by the removal of one hydrogen atom from a single carbon or nitrogen ring atom. Typical heterocyclyl radicals include 2H-pyrrolyl, 2-pyrrolinyl, 3-pyrrolinyl, pyrrolidinyl, 1,3-dioxolanyl, 2-imidazolinyl (also referred to as 4,5-dihydro-1H-imidazolyl), imidazolidinyl, 2-pyrazolinyl, pyrazolidinyl, tetrazolyl, piperidinyl, 1,4-dioxanyl, morpholinyl, 1,4-dithianyl, thiomorpholinyl, piperazinyl, azepanyl, hexahydro-1,4-diazepinyl and the like.

The term “protecting groups” refer to those moieties known in the art that are used to mask functional groups; protecting groups may be removed during subsequent synthetic transformations or by metabolic or other in vivo administration conditions. During any of the processes for preparation of the compounds of the present invention, it may be necessary and/or desirable to protect sensitive or reactive groups on any of the molecules concerned. This may be achieved by means of conventional protecting groups, such as those described in Protective Groups in Organic Chemistry, ed. J. F. W. McOmie, Plenum Press, 1973; and T. W. Greene & P. G. M. Wuts, Protective Groups in Organic Synthesis, Third Edition, John Wiley & Sons, 1999. The protecting groups may be removed at a convenient subsequent stage using methods known in the art.

The term “subject” as used herein, refers to an animal, preferably a mammal, most preferably a human, who has been the object of treatment, observation or experiment.

The term “substituted,” refers to a core molecule on which one or more hydrogen atoms have been replaced with one or more functional radical moieties. Substitution is not limited to a core molecule, but may also occur on a substituent radical, whereby the substituent radical becomes a linking group.

The term “independently selected” refers to one or more substituents selected from a group of substituents, wherein the substituents may be the same or different.

The substituent nomenclature used in the disclosure of the present invention was derived by first indicating the atom having the point of attachment, followed by the linking group atoms toward the terminal chain atom from left to right, substantially as in:
(C₁₋₆)alkylC(O)NH(C₁₋₆)alkyl(Ph)
or by first indicating the terminal chain atom, followed by the linking group atoms toward the atom having the point of attachment, substantially as in:
Ph(C₁₋₆)alkylamido(C₁₋₆)alkyl
either of which refers to a radical of the formula:

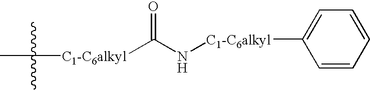

Lines drawn into ring systems from substituents indicate that the bond may be attached to any of the suitable ring atoms.

When any variable (e.g. R₄) occurs more than one time in any embodiment of Formula I, each definition is intended to be independent.

The terms “comprising”, “including”, and “containing” are used herein in their open, non-limited sense.

Nomenclature

Except where indicated, compound names were derived using nomenclature rules well known to those skilled in the art, by either standard IUPAC nomenclature references, such as Nomenclature of Organic Chemistry, Sections A, B, C, D, E, F and H, (Pergamon Press, Oxford, 1979, Copyright 1979 IUPAC) and A Guide to IUPAC Nomenclature of Organic Compounds (Recommendations 1993), (Blackwell Scientific Publications, 1993, Copyright 1993 IUPAC); or commercially available software packages such as Autonom (brand of nomenclature software provided in the ChemDraw Ultra® office suite marketed by CambridgeSoft.com); and ACD/Index Name™ (brand of commercial nomenclature software marketed by Advanced Chemistry Development, Inc., Toronto, Ontario).

Abbreviations

As used herein, the following abbreviations are intended to have the following meanings (additional abbreviations are provided where needed throughout the Specification):

Boc tert-butoxycarbonyl

DMF dimethylformamide

DMSO dimethylsulfoxide

EDCl, or EDC 1-(3-Dimethylaminopropyl)-3-ethylcarbodiimide hydrochloride

EtOAc Ethyl acetate

HOBt 1-hydroxybenzotriazole

MeOH Methyl alcohol

NMR nuclear magnetic resonance

rt room temperature

TFA trifluoroacetic acid

THF tetrahydrofuran

Pharmaceutically Acceptably Salts

The compounds of the present invention may also be present in the form of pharmaceutically acceptable salts.

For use in medicines, the salts of the compounds of this invention refer to non-toxic “pharmaceutically acceptable salts.” FDA approved pharmaceutically acceptable salt forms (Ref. International J. Pharm. 1986, 33, 201-217; J. Pharm. Sci., 1977, January, 66(1), p 1) include pharmaceutically acceptable acidic/anionic or basic/cationic salts.

Pharmaceutically acceptable acidic/anionic salts include, and are not limited to acetate, benzenesulfonate, benzoate, bicarbonate, bitartrate, bromide, calcium edetate, camsylate, carbonate, chloride, citrate, dihydrochloride, edetate, edisylate, estolate, esylate, fumarate, glyceptate, gluconate, glutamate, glycollylarsanilate, hexylresorcinate, hydrabamine, hydrobromide, hydrochloride, hydroxynaphthoate, iodide, isethionate, lactate, lactobionate, malate, maleate, mandelate, mesylate, methylbromide, methylnitrate, methylsulfate, mucate, napsylate, nitrate, pamoate, pantothenate, phosphate/diphosphate, polygalacturonate, salicylate, stearate, subacetate, succinate, sulfate, tannate, tartrate, teoclate, tosylate and triethiodide. Organic or inorganic acids also include, and are not limited to, hydriodic, perchloric, sulfuric, phosphoric, propionic, glycolic, methanesulfonic, hydroxyethanesulfonic, oxalic, 2-naphthalenesulfonic, p-toluenesulfonic, cyclohexanesulfamic, saccharinic or trifluoroacetic acid.

Pharmaceutically acceptable basic/cationic salts include, and are not limited to aluminum, 2-amino-2-hydroxymethyl-propane-1,3-diol (also known as tris(hydroxymethyl)aminomethane, tromethane or “TRIS”), ammonia, benzathine, t-butylamine, calcium, calcium gluconate, calcium hydroxide, chloroprocaine, choline, choline bicarbonate, choline chloride, cyclohexylamine, diethanolamine, ethylenediamine, lithium, LiOMe, L-lysine, magnesium, meglumine, NH₃, NH₄OH, N-methyl-D-glucamine, piperidine, potassium, potassium-t-butoxide, potassium hydroxide (aqueous), procaine, quinine, sodium, sodium carbonate, sodium-2-ethylhexanoate (SEH), sodium hydroxide, triethanolamine (TEA) or zinc.

Prodrugs

The present invention includes within its scope prodrugs of the compounds of the invention. In general, such prodrugs will be functional derivatives of the compounds which are readily convertible in vivo into an active compound. Thus, in the methods of treatment of the present invention, the term “administering” shall encompass the means for treating, ameliorating or preventing a syndrome, disorder or disease described herein with a compound specifically disclosed or a compound, or prodrug thereof, which would obviously be included within the scope of the invention albeit not specifically disclosed for certain of the instant compounds. Conventional procedures for the selection and preparation of suitable prodrug derivatives are described in, for example, “Design of Prodrugs”, ed. H. Bundgaard, Elsevier, 1985.

Stereochemical Isomers

One skilled in the art will recognize that the compounds of Formula I may have one or more asymmetric carbon atoms in their structure. It is intended that the present invention include within its scope: single enantiomer forms of the compounds, diastereomers, racemic mixtures, and mixtures of enantiomers in which an enantiomeric excess is present.

The term “single enantiomer” as used herein defines all the possible homochiral forms which the compounds of Formula I and their N-oxides, addition salts, quaternary amines or physiologically functional derivatives may possess.

Stereochemically pure isomeric forms may be obtained by the application of art known principles. Diastereoisomers may be separated by physical separation methods such as fractional crystallization and chromatographic techniques, and enantiomers may be separated from each other by the selective crystallization of the diastereomeric salts with optically active acids or bases or by chiral chromatography. Pure stereoisomers may also be prepared synthetically from appropriate stereochemically pure starting materials, or by using stereoselective reactions.

The term “isomer” refers to compounds that have the same composition and molecular weight but differ in physical and/or chemical properties. Such substances have the same number and kind of atoms but differ in structure. The structural difference may be in constitution (geometric isomers) or in an ability to rotate the plane of polarized light (enantiomers).

The term “stereoisomer” refers to isomers of identical constitution that differ in the arrangement of their atoms in space. Enantiomers and diastereomers are examples of stereoisomers.

The term “chiral” refers to the structural characteristic of a molecule that makes it impossible to superimpose it on its mirror image.

The term “enantiomer” refers to one of a pair of molecular species that are mirror images of each other and are not superimposable.

The term “diastereomer” refers to stereoisomers that are not mirror images.

The symbols “R” and “S” represent the configuration of substituents around a chiral carbon atom(s).

The term “racemate” or “racemic mixture” refers to a composition composed of equimolar quantities of two enantiomeric species, wherein the composition is devoid of optical activity.

The term “homochiral” refers to a state of enantiomeric purity.

The term “optical activity” refers to the degree to which a homochiral molecule or nonracemic mixture of chiral molecules rotates a plane of polarized light.

The term “geometric isomer” refers to isomers that differ in the orientation of substituent atoms in relationship to a carbon-carbon double bond, to a cycloalkyl ring or to a bridged bicyclic system. Substituent atoms (other than H) on each side of a carbon-carbon double bond may be in an E or Z configuration. In the “E” (opposite sided) configuration, the substituents are on opposite sides in relationship to the carbon-carbon double bond; in the “Z” (same sided) configuration, the substituents are oriented on the same side in relationship to the carbon-carbon double bond. Substituent atoms (other than hydrogen) attached to a carbocyclic ring may be in a cis or trans configuration. In the “cis” configuration, the substituents are on the same side in relationship to the plane of the ring; in the “trans” configuration, the substituents are on opposite sides in relationship to the plane of the ring. Compounds having a mixture of “cis” and “trans” species are designated “cis/trans”.

It is to be understood that the various substituent stereoisomers, geometric isomers and mixtures thereof used to prepare compounds of the present invention are either commercially available, can be prepared synthetically from commercially available starting materials or can be prepared as isomeric mixtures and then obtained as resolved isomers using techniques well-known to those of ordinary skill in the art.

The isomeric descriptors “R,” “S,” “E,” “Z,” “cis,” and “trans” are used as described herein for indicating atom configuration(s) relative to a core molecule and are intended to be used as defined in the literature (IUPAC Recommendations for Fundamental Stereochemistry (Section E), Pure Appl. Chem., 1976, 45:13-30).

The compounds of the present invention may be prepared as individual isomers by either isomer-specific synthesis or resolved from an isomeric mixture. Conventional resolution techniques include forming the free base of each isomer of an isomeric pair using an optically active salt (followed by fractional crystallization and regeneration of the free base), forming an ester or amide of each of the isomers of an isomeric pair (followed by chromatographic separation and removal of the chiral auxiliary) or resolving an isomeric mixture of either a starting material or a final product using preparative TLC (thin layer chromatography) or a chiral HPLC column.

Polymorphs

Furthermore, compounds of the present invention may have one or more polymorph or amorphous crystalline forms and as such are intended to be included in the scope of the invention. In addition, some of the compounds may form solvates with water (i.e., hydrates) or common organic solvents, and such are also intended to be encompassed within the scope of this invention.

Tautomeric Forms

Some of the compounds of Formula I may also exist in their tautomeric forms. Such forms although not explicitly indicated in the present application are intended to be included within the scope of the present invention.

Synthesis

Compounds of the present invention may be made according to traditional organic synthetic methods or by matrix or combinatorial synthetic methods. The representative methods described below are for illustrative purposes only and are in no way meant to limit the invention.

General Guidance

Representative intermediates and compounds of the present invention can be synthesized in accordance with the general synthetic sequences or methods as illustrated in Scheme 1. The invention should not be construed as being limited by the chemical reactions and conditions expressed. The groups R¹, R², R³, R⁴, R⁵, R⁶, R⁷, R⁸, R⁹, and R¹⁰ are as described in Formula I.

In Scheme 1, the R group is an alkyl group, such as methyl, ethyl, tert-butyl, substituted benzyl and the like. The X represents a leaving group such as halogen or a sulfonate ester (e.g. methanesulfonate, toluenesulfonate, or trifluoromethanesulfonate). The group P′ represents a protecting group such as trityl (triphenylmethyl), tert-butyl, substituted benzyl, tetrahydro-2H-pyran-2-yl, or substituted silyl (e.g. trimethylsilyl, or tert-butyldimethylsilyl).

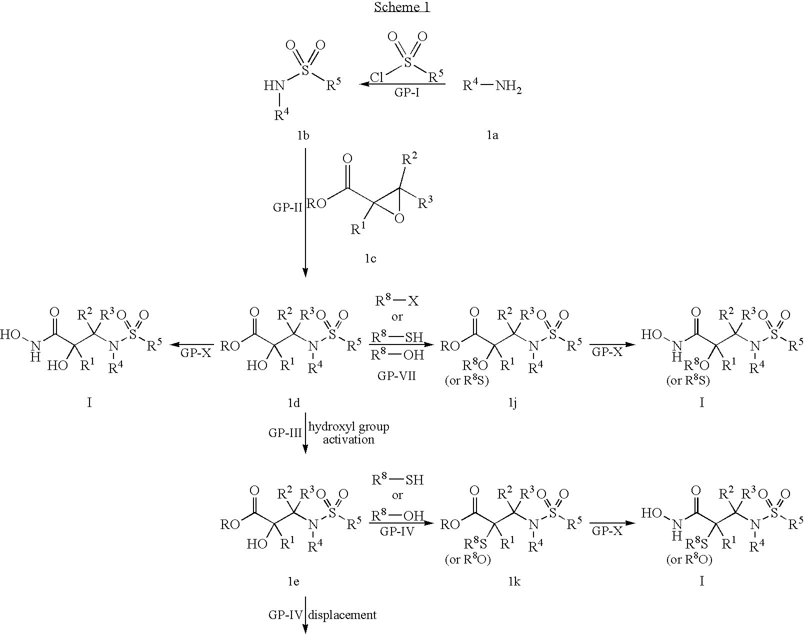

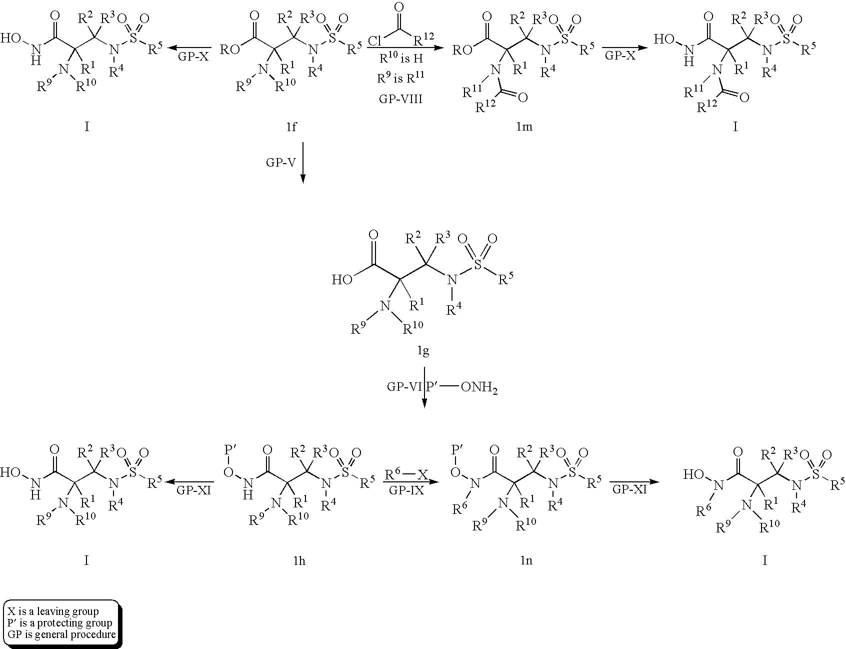

General procedure I (GP-I) describes the formation of sulfonamide intermediate 1b, wherein R⁴ and R⁵ are as described in Formula I. Compound 1a (R⁴—NH₂) is reacted with sulfonyl chloride (R⁵SO₂Cl) in the presence of a base, such as pyridine or triethylamine, in a suitable solvent, such as dichloromethane or chloroform, under an inert atmosphere. Sulfonyl chlorides of the formula (R⁵SO₂Cl) are commercially available. Compounds of formula Ia are either commercially available or they can be prepared by methods described in Scheme 2, Scheme 3, and Scheme 4 herein below.

General procedure II (GP-II) describes the reaction of epoxide 1c with intermediate 1b to provide intermediate 1d, wherein R, R¹, R², R³, R⁴, and R⁵ are as described in Formula I. This reaction can be performed under thermal-heating conditions or under microwave irradiation. For example, intermediate 1d may be obtained by the following thermal heating conditions: a mixture of intermediate 1b, a base (such as metal carbonate, e.g. K₂CO₃, or Cs₂CO₃), and a suitable phase transfer reagent (e.g. benzyltriethylammonium chloride) in a suitable solvent (e.g. 1,4-dioxane) is treated with epoxide 1c (e.g. methyl glycidate, wherein R¹, R², and R³ are the hydrogen and R is methyl). The mixture is sealed and is heated to a suitable temperature, such as 70-90° C. for 1,4-dioxane. (see: Domenico Albanese et al. Ind. Eng. Chem. Res. 2003, 42, 680-686.). Alternatively, intermediate 1d may be obtained by the following microwave irradiation procedure: a mixture of intermediate 1b and a base (such as metal carbonate, e.g. K₂CO₃, or Cs₂CO₃) in a suitable solvent (e.g. DMF or DMSO) is added an epoxide 1c (e.g. methyl glycidate). The mixture is then sealed and is heated at suitable temperature for a suitable period of time (e.g. 80-120° C. for 20-60 min when DMF is used as the solvent) under microwave irradiation.

The epoxide 1c can be either racemic mixture or optical pure material, which are commercially available and/or can be prepared by methods described in Scheme 5 herein below. The intermediate 1d can be converted directly to compounds of Formula I, wherein R⁷ is a hydroxyl group, according to general procedure X (GP-X) as described below.

The activation of the hydroxyl group in 1d is described as general procedure III (GP-III) in Scheme 1. The AO group in intermediate 1e presents the activated hydroxyl group (e.g. A group is trifluoromethanesulfonyl, methanesulfonyl, or 4-toluenesulfonyl). For example, to a solution of intermediate 1d in a suitable solvent (e.g. CH₂Cl₂, or CHCl₃) under an inert gas at a suitable temperature (e.g. −40 to −20° C.) is added a base (e.g. pyridine, or 2,6-lutidine) and an activation reagent such as trifluoromethanesulfonic anhydride (see Timothy P. Kogan et al. Tetrahedron, 1990, 46, 6623.)

The S_{N}2 displacement reaction of intermediate 1e is described as general procedure IV (GP-IV) in Scheme 1. For example, intermediate 1e is treated with nucleophile (e.g. amine (R⁹R¹⁰NH), or R⁸—SH in the presence of a suitable base (e.g. pyridine, or 2,6-lutidine), or R⁸—OH in the presence of a suitable base (e.g. pyridine, or 2,6-lutidine) in a suitable solvent (e.g. CH₂Cl₂, or CHCl₃) at a suitable temperature to provide intermediate 1f or intermediate 1k. The nucleophiles R⁸—SH, R⁸—OH, and R⁹R¹⁰NH, where the R⁸, R⁹, and R¹⁰ are as described herein above, are commercially available or said nucleophiles can be prepared by simple functional transformations that are well known to those skilled in the art. Intermediates 1f and 1k can be converted directly to compounds of Formula I, wherein R⁷ is R⁹R¹⁰N—, R⁸S—, or R⁸O—, using general procedure X (GP-X) as described below.

General procedure V (GP-V) describes the hydrolysis of α-heteroatom substituted alkyl ester 1f into a carboxylic acid 1g. The conditions used are dependent on the nature of R group, wherein R is as described herein above. Typically, basic conditions such as aqueous metal hydroxide (e.g. lithium hydroxide, or sodium hydroxide) together with an organic solvent (e.g. THF, or 1,4-dioxane) is preferred. When R is tert-butyl, acidic conditions, such as trifluoroacetic acid are preferred.

The coupling reaction of carboxylic acid 1g with O-protected hydroxylamine to form intermediate 1h is described as general procedure VI (GP-VI) in Scheme 1. For example, a mixture of intermediate 1g and suitable coupling reagent combination [(e.g. N-(3-dimethylaminopropyl)-N-ethylcarbodiimide hydrochloride (EDCl) and 1-hydroxybenzotriazole (HOBt)] in the presence of a base (e.g. 4-N,N-dimethylaminopyridine) in chlorinated solvent (e.g. CHCl₃) is treated with another base (e.g. triethylamine) under an inert gas. After reaction of the carboxylic acid with the coupling reagents, an O-protected hydroxylamine (e.g. O-tritylhydroxylamine, or O-benzylhydroxylamine) is added to provide intermediate 1h. The protected hydroxylamines (P′-ONH₂), where the P′ group is trityl (triphenylmethyl), benzyl, trimethylsilyl, tert-butyldimethylsilyl, or tetrahydro-2H-pyran-2-yl, are commercially available. The intermediate 1h can be converted directly to compounds of Formula I according to the general procedure XI (GP-XI) as described below.

General procedure VII (GP-VII) describes an alternative method to convert intermediate 1d to intermediate 1j. This reaction may be performed either under base promoted alkylation or under Mitsunobu type conditions. For base promoted alkylation, a mixture of intermediate 1d and an alkylating reagent (R⁸—X, e.g. methyl iodide) in a suitable solvent (e.g. DMF, or DMSO) under inert gas is treated with a base (e.g. sodium hydride) to provide intermediate 1j.

When R¹⁰ is hydrogen and R⁹ is R¹¹, the intermediate 1f can be converted to intermediate 1m, wherein R⁷ is an amide substitutent, according to general procedure VIII (GP-VIII) in Scheme 1. Treatment of intermediate 1f with an acyl chloride [R¹²C(═O)Cl] in the presence of a suitable base (e.g. triethylamine, or 2,6-lutidine) or with the corresponding carboxylic acid [R¹²C(═O)OH] in the presence of coupling reagents [e.g. N-(3-dimethylaminopropyl)-N-ethylcarbodiimide hydrochloride (EDCl) and 1-hydroxybenzotriazole (HOBt)] affords amide 1m. For more specific example, to a solution of intermediate 1f in a solvent (e.g. THF, or CH₂Cl₂) is added a base (e.g. 2,6-lutidine, or triethylamine) followed by the acyl chloride [R¹²C(═O)Cl] to provide intermediate 1m. The acyl chlorides [R¹²C(═O)Cl] are commercially available or they can be prepared from the corresponding carboxylic acid [R¹²C(═O)OH] and SOCl₂.

The alkylation of O-protected hydroxylamide 1h with electrophiles (R⁶—X), wherein R⁶ is as defined in Formula I, to afford intermediate 1n is described as general procedure IX (GP-IX) in Scheme 1. This reaction is typically performed under basic conditions. For example, a mixture of intermediate 1h and a base (such as metal carbonate, e.g. K₂CO₃, or Cs₂CO₃) in a suitable solvent (e.g. DMF, or DMSO) is treated with an electrophile (e.g. 2-methylpropyl iodide) to afford intermediate 1n. The electrophiles (R⁶—X) are commercially available or they can be prepared by activation of corresponding alcohol (R⁶—OH) by methods described very well in the literature. When R⁶ is substituted allyl group, a metal catalyzed substitution can be used. For example, treatment of 1h with a suitable allylating reagent, such as allyl methyl carbonate, and a suitable catalyst, such as Pd(PPh₃)₄, in a suitable solvent, such as MeCN, provides In (see: J. Org. Chem. 2005, 70, 2148; Synlett 2003, 567). The intermediate 1n can be converted directly to compounds of Formula I according to the general procedure XI (GP-XI) as described below.

General procedure X (GP-X) describes the conversion of an ester group to an N-hydroxylamide functionality. The procedure is suitable for conversion of intermediates 1d, 1f, 1j, 1k, or 1m to compounds of Formula I. For example, a mixture of ester (such as intermediate 1d, 1f, 1j, 1k, or 1m) and a suitable hydroxylamine sources (e.g. hydroxylamine hydrochloride salt) in a suitable solvent (e.g. MeOH) is treated with a base (e.g. sodium methoxide, or potassium hydroxide) at a suitable temperature to provide compounds of Formula I.

The deprotection of O-protected N-hydroxylamide 1n or 1h to N-hydroxylamide, such as the compounds of Formula I, is described as general procedure XI (GP-XI) in Scheme 1. The conditions used depend on the nature of protecting group P′. For example, when the protecting group P′ is trityl (triphenylmethyl) group or tetrahydro-2H-pyran-2-yl, acidic conditions (such as trifluoroacetic acid, or BF₃.OEt₂) are preferred. When the protecting group P′ is benzyl, substituted benzyl, and the like, catalytic hydrogenation (e.g. H₂, Pd/C, CH₃OH) can be used as well. When the protecting group P′ is a silyl protecting group, such as trimethylsilyl, tert-bytyldimethylsilyl, and the like, a reagent containing fluoride sources (e.g. tetrabutylammonium fluoride) can be used. All of the conditions for deprotection of P′ group are well documented in the art. (T. W. Greene and P. G. M. Wuts, Protective Groups in Organic Synthesis, 1999, John Wiley & Sons.)

The compounds 1a (R⁴NH₂) with diverse R⁴ groups that described herein above are commercially available and, alternatively, can be synthesized by the methods showed in Scheme 2, Scheme 3, and Scheme 4, wherein the A=B, Y, Z, and R^{a} are as described in Formula I. Scheme 2 illustrates the metal-catalyzed cross-coupling reactions of 2a, where the D is 1, Br or Cl and the G is —NO₂ or —NHBoc (Boc: t-butoxycarbonyl), with suitable boronic acids or boronate esters (Suzuki coupling, where E is —B(OH)₂ or a boronic ester, Chem. Rev. 1995, 95, 2457), or tin reagents (Stille coupling, where E is −Sn(alkyl)₃, Angew. Chem. Int. Ed. 1986, 25, 508; Pure Appl. Chem. 1985, 57.1771) to give compounds 2g-k. The cross-coupling reactions can be performed according to the standard methodology described above, preferably in the presence of a palladium catalyst (such as Pd(PPh₃)₄, or Pd(OAc)₂), a suitable base (such as t-BuOK, KF, NaCO₃ or an organic base such as triethylamine), and a suitable solvent (such as toluene, 1,2-dimethoxyethane, 1,4-dioxane, or DMF) (see: J. Med. Chem. 1997, 40, 3542; Tetrahedron 2005, 61, 7289; J. Org. Chem. 2005, 70, 6122). The reactions can be carried out either under thermal heating conditions or under microwave irradiation. The reduction of nitro group of 2g-k (where G is —NO₂) to the corresponding amino group (—NH₂, compounds 2l-p) can be performed in the presence of SnCl₂ with a suitable solvent, such as ethanol (see: Bioorg. Med. Chem. Lett. 2005, 15, 4985) or using catalytic hydrogenation (e.g. H₂, Pd/C, CH₃OH) (see: Bioorg. Med. Chem. Lett. 2004, 12, 4477). When G is —NHBoc, the removal of Boc group can be achieved under suitable acidic conditions, such as HCl, or trifluoroacetic acid, and a suitable solvent, such as dichloromethane, or ether.

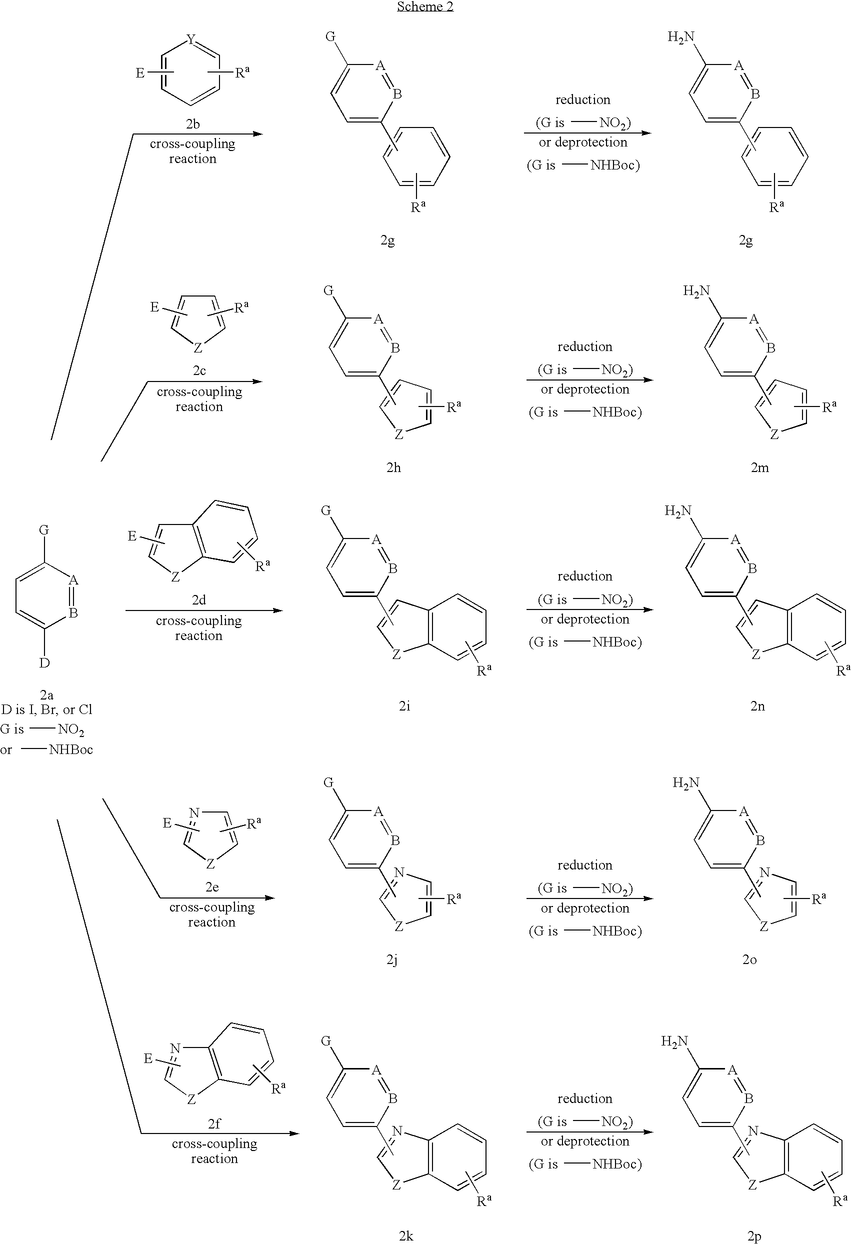

Scheme 3 describes representative methods to synthesize 1a (R⁴NH₂) with an R⁴ group containing a linking group X, where X is O, or S. The Ullmann type cross-coupling of 2a with 3b, where X is O or S, in the presence of suitable catalyst (such as CuI, or Cu₂O), a suitable base (such as Cs₂CO₃), a suitable ligand (such as N,N-dimethylglycine), and a suitable solvent (such as 1,4-dioxane, or acetonitrile) to give compound 3c (see: Org. Lett. 2003, 5, 3799; Org. Lett. 2004, 6, 913; Synlett 2005, 1291). Compound 3c can be, alternatively, synthesized by aromatic nucleophilic substitution reaction (S_{N}Ar reaction) from 3a and 3b in the presence of a suitable base (such as NaH, or Cs₂CO₃) in a suitable solvent (such as DMSO, or DMF) (see: Bioorg. Med. Chem. Lett. 2005, 15, 4985; Bioorg. Med. Chem. Lett. 2004, 12, 4477). The reduction of nitro group of 3c to the corresponding amino group (—NH₂, compound 3d) can be performed according to the similar procedure for synthesis of compounds 2l-p as described in Scheme 2.

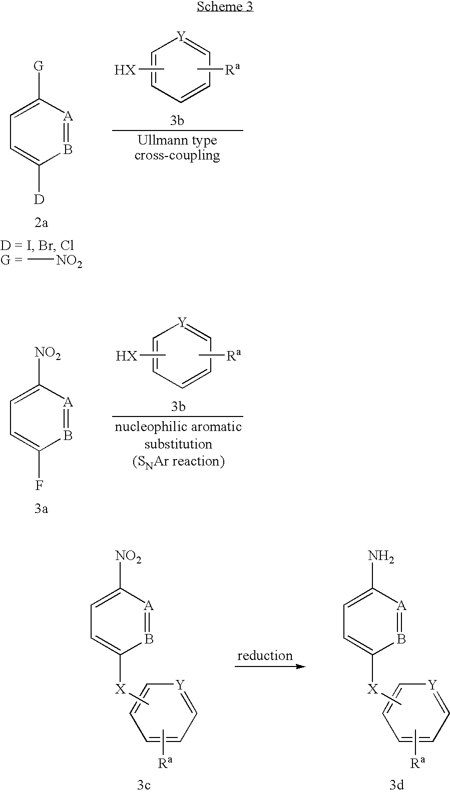

Scheme 4 illustrates the synthesis of 1a (R⁴NH₂) wherein X is an acetylenyl linking group. The Sonogashira coupling of 2a (G is —NHBoc) with 4a-d can be performed in the presence of a suitable catalyst (such as Pd(OAc)₂, PdCl₂, or PdCl₂(PPh₃)₂), a suitable base (such as Cs₂CO₃ or organic base, such as triethylamine, or pyrrolidine), and a suitable solvent (such as acetonitrile, or THF) to give 4f-i (see: J. Org. Chem. 2005, 70, 4393; J. Org. Chem. 2005, 70, 4869; Synthesis 2005, 804; Tetrahedron 2003, 59, 8555; Chem. Commun. 2004, 514; J. Am. Chem. Soc. 2003, 125, 6753). A co-catalyst, such as CuI can be optionally added. The Suzuki coupling or Stille coupling of 2a (G is —NHBoc) with 4e, where E is boronic acid (—B(OH)₂), boronic ester, or —Sn(alkyl)₃, can be performed to give 4j according to the procedure described above for the synthesis of 2g-k in Scheme 2. The removal of the Boc group from 4f-j to give 4k-o can be achieved under a suitable acidic condition, such as HCl, or trifluoroacetic acid, and a suitable solvent, such as dichloromethane, or ether.

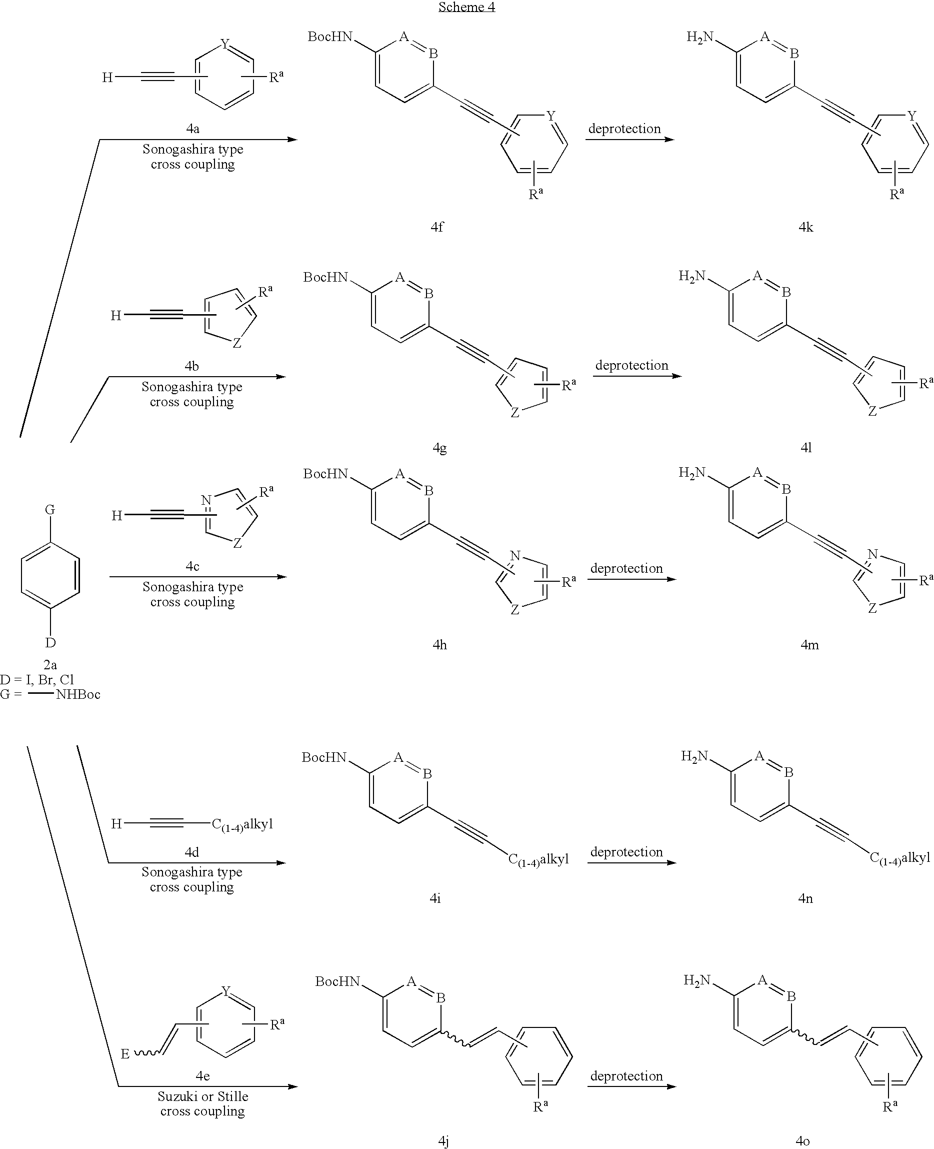

The compound 1c is commercially available or can be, alternatively, synthesized by epoxidation as described in Scheme 5. The epoxidation of 5a can be performed in a suitable solvent (such as methylene chloride) with a suitable oxidative reagent (such as t-BuOOH, m-chlorobenzoic peracid, or dimethyldioxirane) to give 1c (see: Tetrahedron Lett. 2004, 45, 5359; J. Org. Chem. 1995, 60, 3887; Tetrahedron Lett. 1993, 34, 2469; Tetrahedron Lett. 1990, 31, 331). The epoxide 1c can be either racemic mixture or optically pure material prepared by asymmetric epoxidation methods well know in the literature (see: J. Am. Chem. Soc. 2005, 127, 8962; J. Am. Chem. Soc. 2002, 124, 8792).

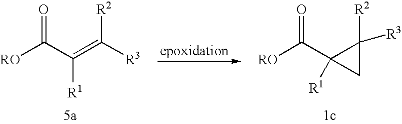

### REFERENCE EXAMPLES

The reference examples described below provide procedures for the formation of intermediates or precursors, which are for the preparation of compounds for the present invention, in accordance with the synthetic sequences appeared in Scheme 1. The reference examples refer to the general procedure I to general procedure IX (GP-I to GP-IX).

### Reference Example for General Procedure I

### GP-I: Formation of Intermediate 1b

### N-[4-(4-trifluoromethylphenoxy)phenyl]methanesulfonamide. (1b-1)

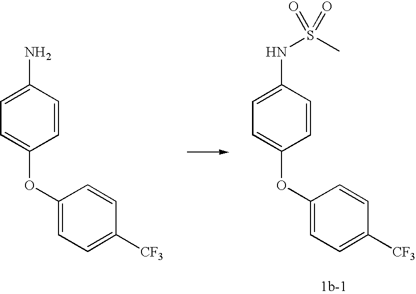

To a solution of 4-(4-trifluoromethylphenoxy)aniline (15.18 g, 60 mmol) and pyridine (7.2 mL, 90 mmol) in dried CH₂Cl₂ (100 mL) under N₂ at 0° C. was added methanesulfonyl chloride (5.22 mL, 66 mmol) dropwise. The mixture was stirred at 0° C. for 1 h and was then poured into CH₂Cl₂/H₂O (100 mL/100 mL). 2N HCl_{(aq)} (30 mL) was added into additional funnel. The organic layer was then washed with H₂O (100 mL), brine (100 mL), dried (Na₂SO₄), and filtered. After removal of solvent, the crude product can be resolidified from Et₂O/hexane to give 18.2 g of N-[4-(4-trifluoromethylphenoxy)phenyl]methanesulfonamide (92%) as a pale brown solid. ¹H NMR (300 MHz, CDCl₃) δ 7.58 (d, J=9.0 Hz, 2H), 7.30 (d, J=9.0 Hz, 2H), 7.04 (d, J=9.0 Hz, 4H), 6.93 (s, 1H, NH), 3.04 (s, 3H); MS (EI, m/z): 331 (M⁺), 330 (M⁺−1, 100).

### Reference Example for General Procedure II

### GP-II: Formation of Intermediate 1d

### Methyl (2R)-2-hydroxy-3-{N-[4-(4-trifluoromethylphenoxy)phenyl],N-methanesulfonylamino}propionate (1d-1 Method A)

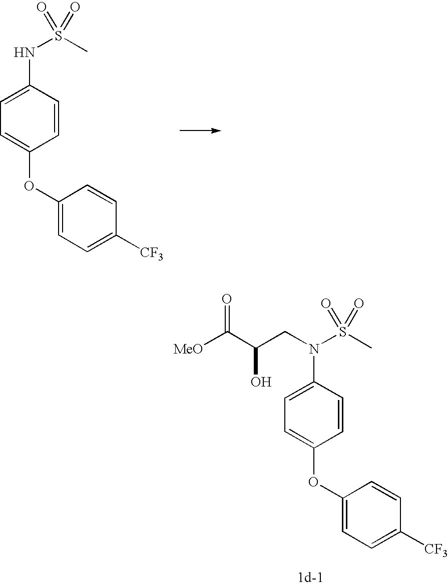

To a mixture of N-[4-(4-trifluoromethylphenoxy)phenyl]methanesulfonamide (16.55 g, 50 mmol), K₂CO₃ (17.3 g, 125 mmol), and benzyltriethylammonium chloride (1.135 g, 5 mmol) in dried 1,4-dioxane (75 mL) was added methyl (R)-glycidate (15.3 g, 150 mmol). The mixture was sealed and was heated to 70° C. for 24 h. The mixture was then poured into Et₂O/H₂O (200 mL/200 mL). The organic layer was washed with brine (200 mL), dried (Na₂SO₄), and filtered. After removal of solvent, the crude product was recrystallized from Et₂O/hexane to give 17.8 g of Methyl (2R)-2-hydroxy-3-{N-[4-(4-trifluoromethylphenoxy)phenyl]-N-methanesulfonylamino}propionate (83%) as a pale brown solid. ¹H NMR (300 MHz, CDCl₃) δ 7.62 (d, J=9.0 Hz, 2H), 7.40 (d, J=9.0 Hz, 2H), 7.10 (d, J=9.0 Hz, 2H), 7.07 (d, J=9.0 Hz, 2H), 4.29 (d, J=6.0 Hz, 1H), 4.03-3.97 (m, 2H), 3.72 (s, 3H), 3.06 (s, 1H, OH), 3.05 (s, 3H); MS (EI, m/z): 456 (M⁺+Na, 100), 434 (M⁺+1). Anal. Calcd for C₁₈H₁₈F₃NO₆S: C, 49.88; H, 4.19; N, 3.23. Found: C, 50.00; H, 4.02; N, 3.22.

### Methyl 2-hydroxy-2-methyl-3-{N-[4-(4-methylphenoxy)phenyl],N-methanesulfonylamino}propionate. (1d-2. Method B).

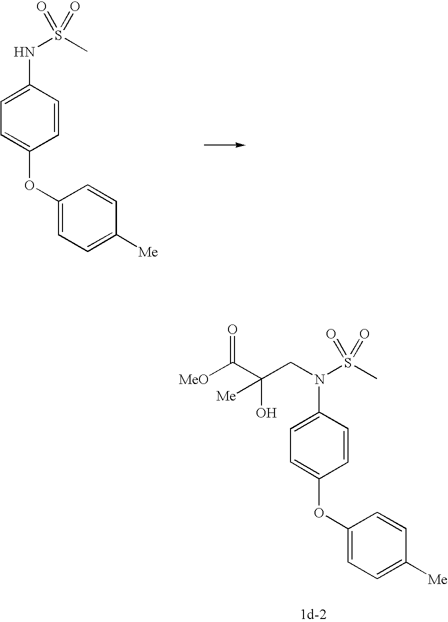

The compound, N-[4-(4-methylphenoxy)phenyl]methanesulfonamide, was prepared from 4-(4-methylphenoxy)aniline in accordance with the general procedure I (GP-I) as described herein above for reference examples.

To a mixture of N-[4-(4-methylphenoxy)phenyl]methanesulfonamide (138 mg, 0.5 mmol) and K₂CO₃ (138 mg, 1.0 mmol) in DMF (1 mL) was added methyl 2-methylglycidate (116 mg, 1.0 mmol). The mixture was sealed and was heated at 120° C. for 20 min under microwave irradiation. The mixture was poured into H₂O/Et₂O (20 mL/20 mL). The organic layer was washed with saturated NH₄Cl_{(aq)} (20 mL), brine (20 mL), dried (Na₂SO₄), and filtered. After removal of solvent, the crude product was purified by silica gel chromatography using acetone/hexane (1/4 to 3/7) as eluent to give 173 mg of Methyl 2-hydroxy-2-methyl-3-{N-[4-(4-methylphenoxy)phenyl]-N-methanesulfonylamino}propionate (88%) as a pale brown solid. ¹H NMR (300 MHz, CDCl₃) δ 7.26-7.21 (m, 2H), 7.17 (d, J=9.0 Hz, 2H), 6.97-6.91 (m, 4H), 4.01 (d, J=12.0 Hz, 1H), 3.94 (d, J=12.0 Hz, 1H), 3.53 (s, 3H), 3.36 (s, 1H, OH), 2.94 (s, 3H), 2.35 (s, 3H), 1.38 (s, 3H); MS (EI, m/z): 416 (M⁺+Na, 100), 393 (M⁺+1).

### Reference Example for General Procedure III

### GP-III: Formation of Intermediate 1e

### Methyl (2R)-2-trifluoromethanesulfonyloxy-3-{N-[4-(4-trifluoromethylphenoxy)phenyl],N-methanesulfonylamino}propionate. (1e-1)

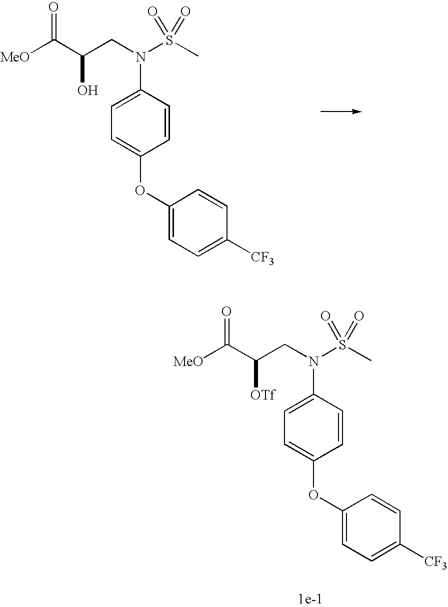

To a solution of Methyl (2R)-2-hydroxy-3-{N-[4-(4-trifluoromethylphenoxy)phenyl]-N-methanesulfonylamino}propionate (24 g, 55.4 mmol) in dried CH₂Cl₂ (100 mL) under N₂ at ca. −20° C. was added 2,6-lutidine (9.6 mL, 83 mmol) and then methanesulfonic anhydride (10.24 mL, 61 mmol) dropwise. The mixture was stirred for 1 h and was then poured into CH₂Cl₂/H₂O (200 mL/200 mL). 2N HCl_{(aq)} (25 mL) was added into the extraction funnel. The organic layer was washed with H₂O (200 mL), dried (Na₂SO₄), and filtered. After removal of solvent, the crude product can be recrystallized from Et₂O/hexane to give 28.2 g of Methyl (2R)-2-trifluoromethanesulfonyloxy-3-{N-[4-(4-trifluoromethylphenoxy)phenyl]-N-methanesulfonylamino}propionate (90%) as a pale brown solid. ¹H NMR (300 MHz, CDCl₃) δ 7.63 (d, J=9.0 Hz, 2H), 7.39 (d, J=9.0 Hz, 2H), 7.12-7.07 (m, 4H), 5.31 (dd, J=6.0, 3.0 Hz, 1H), 4.34-4.20 (m, 2H), 3.82 (s, 3H), 2.98 (s, 3H).

### Reference Example for General Procedure IV

### GP-IV: Formation of Intermediate 1f

### Methyl (2S)-2-(morpholin-4-yl)-3-{N-[4-(4-trifluoromethylphenoxy)phenyl],N-methanesulfonylamino}propionate. (1f-1)

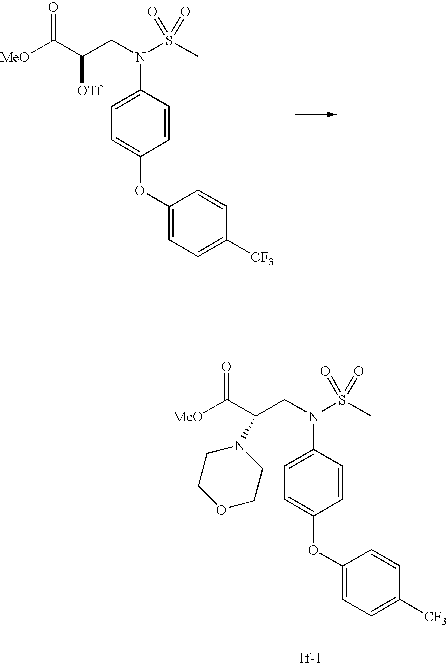

To a solution of Methyl (2R)-2-trifluoromethanesulfonyloxy-3-{N-[4-(4-trifluoromethylphenoxy)phenyl]-N-methanesulfonylamino}propionate (14.12 g, 25 mmol) in dried CH₂Cl₂ (50 mL) at 0° C. under N₂ was added morpholine (6.52 mL, 75 mmol) dropwise. The mixture was stirred for 1 h and was allowed to warm to room temperature. The mixture was poured into CH₂Cl₂/H₂O (50 mL/100 mL). The organic layer was washed with H₂O (100 mL×2), dried (Na₂SO₄), and filtered. After removal of solvent, the mixture was purified by a short path of silica gel using EtOAc/hexane (3/7 to 2/3) as the eluent to give 12 g of Methyl (2S)-2-(morpholin-4-yl)-3-{N-[4-(4-trifluoromethylphenoxy)phenyl]-N-methanesulfonylamino}propionate (95%) as a sticky oil. ¹H NMR (300 MHz, CDCl₃) δ 7.62 (d, J=9.0 Hz, 2H), 7.7.35 (d, J=9.0 Hz, 2H), 7.11-7.04 (m, 4H), 4.05 (dd, J=15.0, 9.0 Hz, 1H), 4.84 (dd, J=15.0, 6.0 Hz, 1H), 3.70 (s, 3H), 3.68-3.54 (m, 4H), 3.36 (dd, J=9.0, 6.0 Hz, 1H), 2.96 (s, 3H), 2.77-2.70 (m, 2H), 2.48-2.41 (m, 2H); MS (EI, m/z): 525 (M⁺+Na), 503 (M⁺+1, 100).

### Reference Example for General Procedure V

### GP-V: Formation of Intermediate 1g

### (2S)-2-(Morpholin-4-yl)-3-{N-[4-(4-trifluoromethylphenoxy)phenyl],N-methanesulfonylamino}propionic acid. (1g-1)

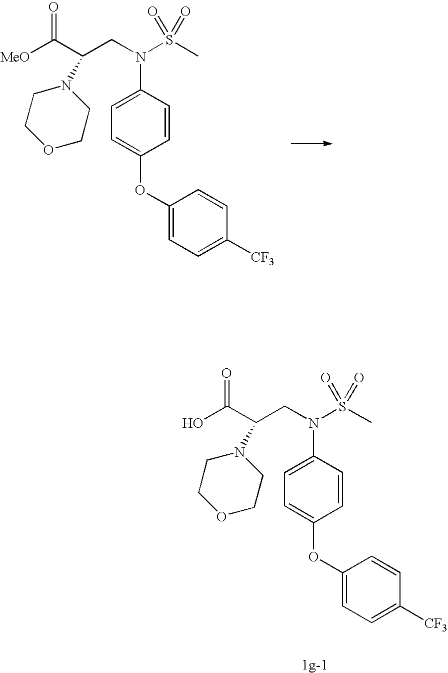

To a mixture of Methyl (2S)-2-(morpholin-4-yl)-3-{N-[4-(4-trifluoromethylphenoxy)phenyl]-N-methanesulfonylamino}propionate (11.55 g, 23 mmol) in THF (46 mL) was added lithium hydroxyl (1.0 M in H₂O, 46 mL, 46 mmol) at room temperature. The mixture was stirred at rt for 2 h. Then, HCl_{(aq)} (2 N, 23 mL, 46 mmol) and hexane (450 mL) were added sequentially. The resulting solid was filtered and was washed with H₂O (50 mL) and hexane (100 mL). The solid was then dried to give 11.0 g of (2S)-2-(Morpholin-4-yl)-3-{N-[4-(4-trifluoromethylphenoxy)phenyl]-N-methanesulfonylamino}propionic acid (98%) as a pale brown solid. ¹H NMR (300 MHz, d₆-acetone) δ 7.76 (d, J=9.0 Hz, 2H), 7.61 (d, J=9.0 Hz, 2H), 7.24-7.15 (m, 4H), 4.10 (dd, J=12.0, 9.0 Hz, 1H), 3.95 (dd, J=12.0, 6.0 Hz, 1H), 3.63-3.48 (m, 4H), 3.38 (dd, J=9.0, 6.0 Hz, 1H), 3.08 (s, 3H), 2.80-2.73 (m, 2H), 2.54-2.47 (m, 2H); MS (EI, m/z): 489 (M⁺+1, 100).

### Reference Example for General Procedure VI

### GP-VI: Formation of Intermediate 1h

### N-Triphenylmethoxy-(2S)-2-(morpholin-4-yl)-3-{N-[4-(4-trifluoromethylphenoxy)phenyl],N-methanesulfonylamino}propionamide. (1h-1)

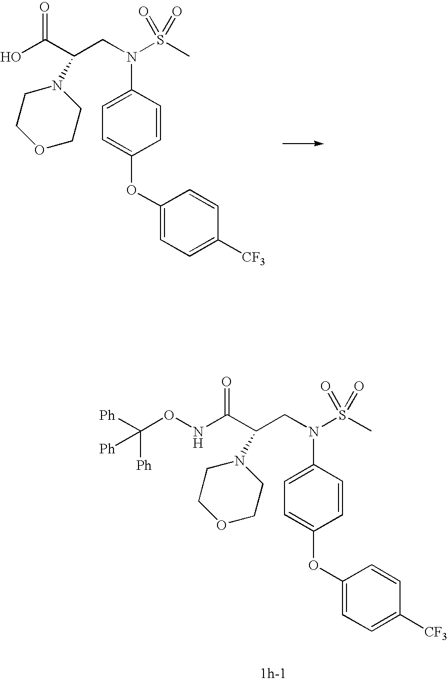

To a mixture of (2S)-2-(Morpholin-4-yl)-3-{N-[4-(4-trifluoromethylphenoxy)phenyl]-N-methanesulfonylamino}propionic acid (10.74 g, 22 mmol), EDC (6.3 g, 33 mmol), HOBt (4.46 g, 33 mmol), and 4-N,N-dimethylaminopyridine (4.03 g, 33 mmol) in CHCl₃ (100 mL) was added Et₃N (4.6 mL, 33 mmol) under N₂ at room temperature. After 1 h stirring, O-tritylhydroxylamine (7.56 g, 27.5 mmol) was added and was stirred for 24 h at rt. The mixture was then poured into CH₂Cl₂/H₂O (50 mL/100 mL). The organic layer was washed with H₂O (100 mL×2), dried (Na₂SO₄), and filtered. After removal of solvent, the crude mixture was purified by silica gel chromatography using EtOAc/hexane (2/3 to 7/3) as eluent to give 12 g of N-Triphenylmethoxy-(2S)-2-(morpholin-4-yl)-3-{N-[4-(4-trifluoromethylphenoxy)phenyl]-N-methanesulfonylamino}propionamide (73%) as a sticky oil. ¹H NMR (300 MHz, CDCl₃) δ 8.82 (s, 1H), 7.61 (d, J=9.0 Hz, 2H), 7.47-7.19 (m, 17H), 7.08 (d, J=9.0 Hz, 2H), 7.02 (d, J=9.0 Hz, 2H), 4.03-4.93 (m, 2H), 3.38-3.27 (m, 4H), 3.16 (dd, J=9.0, 6.0 Hz, 1H), 2.85 (s, 3H), 2.53-2.37 (m, 4H); MS (EI, m/z): 768 (M⁺+Na), 745 (M⁺+1), 243 (100).

### Reference Example for General Procedure VII

### GP-VII: Formation of Intermediate 1j

### Methyl (2S)-2-methoxy-3-{N-[4-(4-methylphenoxy)phenyl],N-methanesulfonylamino}propionate. (1j-1)

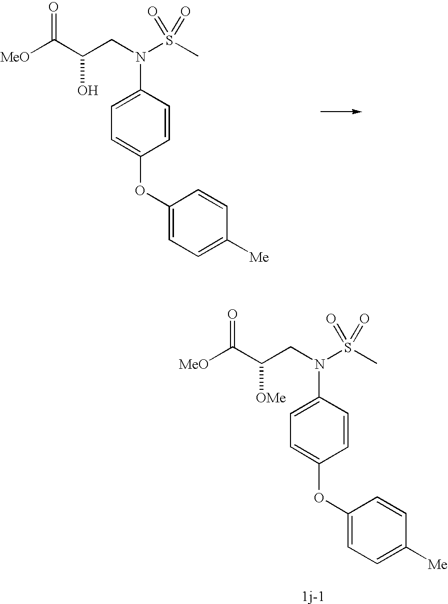

The compound, Methyl (2S)-2-hydroxy-3-{N-[4-(4-methylphenoxy)phenyl]-N-methanesulfonylamino}propionate, was prepared from 4-(4-methylphenoxy)aniline in accordance with the sequence of general procedure I and II (GP-I and GP-II) as described herein above for reference examples.

To a mixture of Methyl (2S)-2-hydroxy-3-{N-[4-(4-methylphenoxy)phenyl]-N-methanesulfonylamino}propionate (180 mg, 0.5 mmol) and methyl iodide (355 mg, 2.5 mmol) in dried DMF (5 mL) under N₂ at room temperature was added sodium hydride (60% in mineral oil, 100 mg, 2.5 mmol). The mixture was stirred at rt for 30 min and was poured into Et₂O/H₂O (100 mL/100 mL). The organic layer was washed with saturated NH₄Cl_{(aq)} (100 mL), brine (100 mL), dride (Na₂SO₄), and filtered. After removal of solvent, the crude product was purified by silica gel chromatography using EtOAc/hexane (1/4 to 1/1) as the eluent to give 108 mg of Methyl (2S)-2-methoxy-3-{N-[4-(4-methylphenoxy)phenyl]-N-methanesulfonylamino}propionate (55%). ¹H NMR (400 MHz, CDCl₃) δ 7.25 (d, J=8.0 Hz, 2H), 7.17 (d, J=8.0 Hz, 2H), 6.98-6.93 (m, 4H), 3.98-3.88 (m, 3H), 3.69 (s, 3H), 3.42 (s, 3H), 2.99 (s, 3H), 2.35 (s, 3H); MS (EI, m/z): 416 (M⁺+Na), 394 (M⁺+1).

### Reference Example for General Procedure VIII

### GP-VIII: Formation of Intermediate 1m

### Methyl (2S)-2-[(tetrahydropyran-4-yl)carbonylamino]-3-{N-[4-(4-trifluoromethylphenoxy)phenyl],N-methanesulfonylamino}propionate. (1m-1)

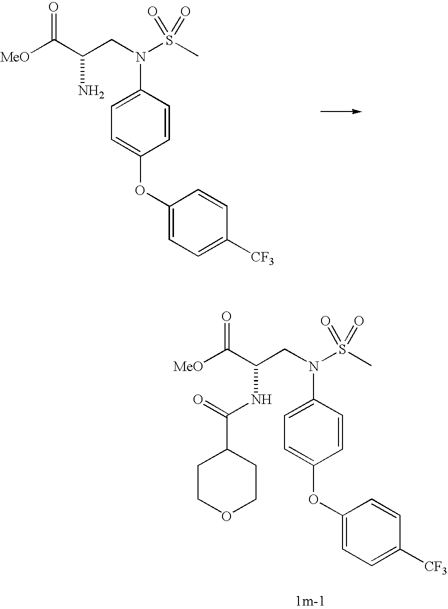

The compound, Methyl (2S)-2-amino-3-{N-[4-(4-trifluoromethylphenoxy)phenyl]-N-methanesulfonylamino}propionate, was prepared from 4-(4-trifluoromethylphenoxy)aniline in accordance with the sequence of general procedure I, II, III, and IV (GP-I, GP-II, GP-III, and GP-IV) as described herein above for reference examples.

To a solution of Methyl (2S)-2-amino-3-{N-[4-(4-trifluoromethylphenoxy)phenyl]-N-methanesulfonylamino}propionate (86 mg, 0.2 mmol) in CH₂Cl₂/THF (1.5 mL/1.5 mL) was added 2,6-lutidine (0.1 mL, 1.2 mmol) and then 4-tetropyranecarboxyl chloride (119 mg, 0.8 mmol) at 0° C. The mixture was allowed to warm to rt and stirred for 1 h. The mixture was poured into Et₂O/H₂O (100 mL/100 mL) and HCl_{(aq)} (2 N, 5 mL) was added. The organic was washed with NaOH_{(aq)} (10%, 10 mL), H₂O (40 mL), brine (50 mL), dried (Na₂SO₄), and filtered. After removal of solvent, the product was dried in vacuo to give 102 mg of Methyl (2S)-2-[(tetrahydropyran-4-yl)carbonylamino]-3-{N-[4-(4-trifluoromethylphenoxy)phenyl]-N-methanesulfonylamino}propionate (94%) as a white solid. ¹H NMR (300 MHz, CDCl₃) δ 7.62 (d, J=9.0 Hz, 2H), 7.33 (d, J=9.0 Hz, 2H), 7.12-7.05 (m, 4H), 6.45 (d, J=9.0 Hz, 1H), 4.68-4.62 (m, 1H), 4.17-3.98 (m, 4H), 3.59 (s, 3H), 3.47-3.38 (m, 2H), 2.90 (s, 3H), 2.42-2.35 (m, 1H), 1.80-1.76 (m, 4H); MS (EI, m/z): 545 (M⁺+1, 100).

### Reference Example for General Procedure IX

### GP-IX: Formation of Intermediate 1n

### N-(2-Methylpropyl),N-triphenylmethoxy(2S)-2-(morpholin-4-yl)-3-{N-[4-(4-trifluoromethylphenoxy)phenyl],N-methanesulfonylamino}propionamide. (1n-1)

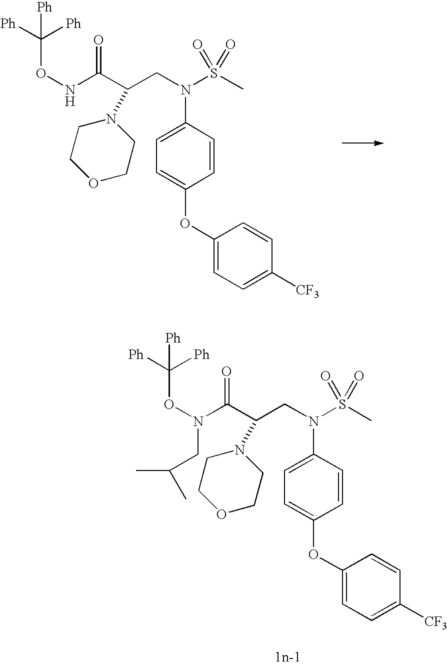

To a mixture of N-Triphenylmethoxy (2S)-2-(morpholin-4-yl)-3-{N-[4-(4-trifluoromethylphenoxy)phenyl],N-methanesulfonylamino}propionamide (149 mg, 0.2 mmol) and Cs₂CO₃ (130 mg, 0.4 mmol) in MeCN/DMF (1 mL/1 mL) was added 2-methyl-1-propyl iodide (110 mg, 0.6 mmol) at room temperature. The mixture was stirred at rt for 5 h and was poured into Et₂O/H₂O (50 mL/50 mL). The organic layer was washed with H₂O (50 mL), brine (50 mL), dried (Na₂SO4), and filtered. After removal of solvent, the crude product was purified by silica gel chromatography using EtOAc/hexane (1/4 to 1/1) as the eluent to give 120 mg of N-(2-Methylpropyl),N-triphenylmethoxy (2S)-2-(morpholin-4-yl)-3-{N-[4-(4-trifluoromethylphenoxy)phenyl],N-methanesulfonylamino}propionamide (75%) as a white solid. ¹H NMR (300 MHz, CDCl₃) δ 7.60 (d, J=9.0 Hz, 2H), 7.36-7.19 (m, 15H), 7.14 (d, J=9.0 Hz, 2H), 7.11 (d, J=9.0 Hz, 2H), 6.94 (d, J=9.0 Hz, 2H), 4.38 (dd, J=9.0, 6.0 Hz, 1H), 4.23 (dd, J=9.0, 6.0 Hz, 1H), 3.68-3.41 (m, 6H), 3.06 (t, J=6.0 Hz, 1H), 2.81 (s, 3H), 2.39-2.20 (m, 4H), 1.92 (quint, J=6.0 Hz, 1H), 0.94 (d, J=6.0 Hz, 3H), 0.93 (d, J=6.0 Hz, 3H); MS (EI, m/z): 824 (M⁺+Na), 802 (M⁺+1, 100).

### EXAMPLES

The detail preparation procedures of examples of this invention were provided below. All of intermediates and precursors for formation of examples of Formula (I) were prepared in a similar manner or similar synthetic sequences as described herein above for reference examples.

### Example 1

### N-Hydroxy (2S)-2-hydroxy-3-{N-[4-(4-chlorophenoxy)phenyl],N-methanesulfonylamino}propionamide

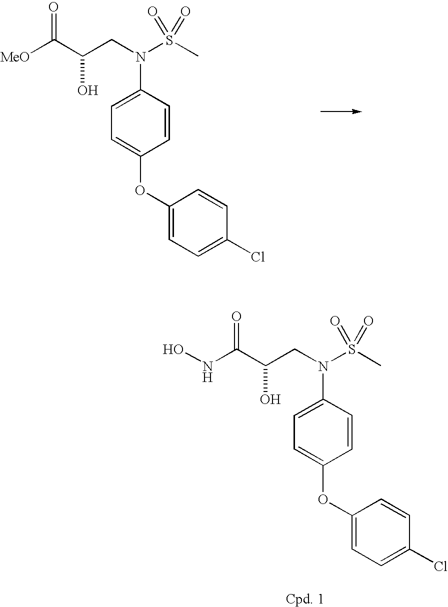

The compound, Methyl (2S)-2-hydroxy-3-{N-[4-(4-chlorophenoxy)phenyl],N-methanesulfonylamino}propionate, was prepared from 4-(4-chlorophenoxy)aniline in accordance with the sequence of general procedure I and II (GP-I and GP-II) as described herein above for reference examples.

To a mixture of Methyl (2S)-2-hydroxy-3-{N-[4-(4-chlorophenoxy)phenyl],N-methanesulfonylamino}propionate (100 mg, 0.25 mmol) and hydroxylamine hydrochloride (69.5 mg, 1.0 mmol) in dried MeOH (1 mL) under N₂ at room temperature was added sodium methoxide (0.5 M in MeOH, 3.0 mL, 1.5 mmol). The mixture was stirred at room temperature for 1 h and was poured into EtOAc/H₂O (100 mL/100 mL) and HCl_{(aq)} (2.0 N, 5.0 mL) was added. The organic layer was washed with H₂O (100 mL), dried (Na₂SO₄) and filtered. After removal of solvent, the product was resolidified from CH₂Cl₂/acetone/hexane (1/1/8) to give 90 mg of N-Hydroxy (2S)-2-hydroxy-3-{N-[4-(4-chlorophenoxy)phenyl],N-methanesulfonylamino}propionamide (90%) as a white solid. ¹H NMR (300 MHz, d₆-DMSO) δ 10.60 (s, 1H), 8.76 (s, 1H), 7.46 (d, J=9.0 Hz, 2H), 7.43 (d, J=9.0 Hz, 2H), 7.08 (d, J=9.0 Hz, 2H), 7.04 (d, J=9.0 Hz, 2H), 5.72 (d, J=6.0 Hz, 1H), 3.86-3.65 (m, 3H), 3.02 (s, 3H); MS (EI, m/z): 423 (M⁺+Na), 399 (M⁺−1, 100). Anal. Calcd for C₁₆H₁₇ClN₂O₆S: C, 47.94; H, 4.27; N, 6.99. Found: C, 47.80; H, 4.19; N, 6.78.

### Example 2

### N-Hydroxy (2S)-2-methoxy-3-{N-[4-(4-methylphenoxy)phenyl],N-methanesulfonylamino}propionamide

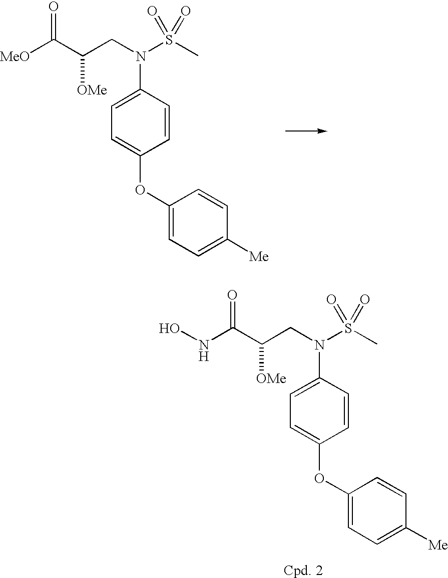

The compound, Methyl (2S)-2-methoxy-3-{N-[4-(4-chlorophenoxy)phenyl],N-methanesulfonylamino}propionate, was prepared from 4-(4-methylphenoxy)aniline in accordance with the sequence of general procedure I, II, and VII (GP-I, GP-II, and GP-VII) as described herein above for reference examples.

To a mixture of Methyl (2S)-2-methoxy-3-{N-[4-(4-chlorophenoxy)phenyl],N-methanesulfonylamino}propionate (98.3 mg, 0.25 mmol) and hydroxylamine hydrochloride (69.5 mg, 1.0 mmol) in dried MeOH (2 mL) under N₂ at room temperature was added sodium methoxide (0.5 M in MeOH, 3.0 mL, 1.5 mmol). The mixture was stirred at room temperature for 1 h and was poured into EtOAc/H₂O (100 mL/100 mL) and HCl (aq) (2.0 N, 6.0 mL) was added. The aqueous layer was extracted with EtOAc (50 mL). The combined organic layers was washed with brine (100 mL), dried (Na₂SO₄) and filtered. After removal of solvent, the product was resolidified from CH₂Cl₂/hexane (2/8) to give 84 mg of N-Hydroxy (2S)-2-methoxy-3-{N-[4-(4-methylphenoxy)phenyl],N-methanesulfonylamino}propionamide (85%) as a white solid. ¹H NMR (400 MHz, d₆-DMSO) δ 10.83 (d, J=4.0 Hz, 1H), 8.94 (d, J=4.0 Hz, 1H), 7.36 (d, J=8.0 Hz, 2H), 7.23 (d, J=8.0 Hz, 2H), 6.99-6.96 (m, 4H), 3.80 (dd, J=12.0, 8.0 Hz, 1H), 3.72 (dd, J=12.0, 4.0 Hz, 1H), 3.51 (dd, J=8.0, 4.0 Hz, 1H), 3.19 (s, 3H), 3.00 (s, 3H), 2.31 (s, 3H); MS (EI, m/z): 417 (M⁺+Na), 395 (M⁺+1, 100). Anal. Calcd for C₁₈H₂₂N₂O₆S: C, 54.81; H, 5.62; N, 7.10. Found: C, 54.89; H, 5.62; N, 7.02.

### Example 3

### N-Hydroxy (2S)-2-(morpholin-4-yl)-3-{N-[4-(4-trifluoromethylphenoxy)phenyl],N-methanesulfonylamino}propionamide

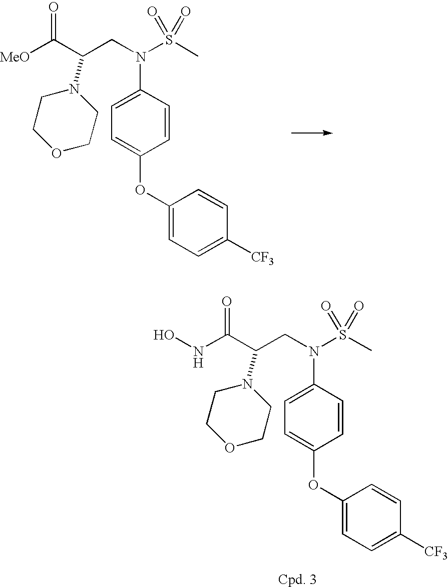

The compound, Methyl (2S)-2-(morpholin-4-yl)-3-{N-[4-(4-trifluoromethylphenoxy)phenyl],N-methanesulfonylamino}propionate, was prepared from 4-(4-trifluoromethylphenoxy)aniline in accordance with the sequence of general procedure I, II, III and IV (GP-I, GP-II, GP-III, and GP-IV) as described herein above for reference examples.

To a mixture of Methyl (2S)-2-(morpholin-4-yl)-3-{N-[4-(4-trifluoromethylphenoxy)phenyl],N-methanesulfonylamino}propionate (936 mg, 1.86 mmol) and hydroxylamine hydrochloride salt (518 mg, 7.45 mmol) in dried MeOH (3 mL) was added sodium methoxide (0.5 M in MeOH, 22.3 mL, 11.2 mmol) at room temperature. The mixture was stirred at rt for 30 h and was poured into EtOAc/H₂O (100 mL/100 mL). The aqueous layer was extracted with EtOAc (50 mL×3). The combined organic layers were dried (Na₂SO₄) and filtered. After removal of solvent, the crude product was dissolved in THF and trifluoroacetic acid (6 mmol) was added. The mixture was stirred for 10 min and the solvent was removed to give a crude mixture, which was purified by reverse HPLC to give 490 mg of N-Hydroxy (2S)-2-(morpholin-4-yl)-3-{N-[4-(4-trifluoromethylphenoxy)phenyl],N-methanesulfonylamino}propionamide (43%) as a white trifluoroacetic acid salt. ¹H NMR (300 MHz, d₆-DMSO) δ 10.80 (br s, 1H), 7.77 (d, J=6.0 Hz, 2H), 7.46 (d, J=9.0 Hz, 2H), 7.19 (d, J=9.0 Hz, 2H), 7.16 (d, J=9.0 Hz, 2H), 4.10-4.00 (m, 1H), 3.87-3.80 (m, 1H), 3.65-3.45 (m, 4H), 3.02 (s, 3H), 3.16-3.10 (m, 1H), 2.75-2.60 (m, 4H); MS (EI, m/z): 526 (M⁺+Na), 504 (M⁺+1, 100). Anal. Calcd for C₂₁H₂₄F₃N₃O₆S·1.0 TFA: C, 44.74; H, 4.08; N, 6.80. Found: C, 45.59; H, 4.18; N, 7.10.

### Example 4

### N-Hydroxy (2S)-3-{N-[4-(4-methylphenoxy)phenyl],N-methanesulfonylamino}-2-(2-propylcarbonylamino)propionamide

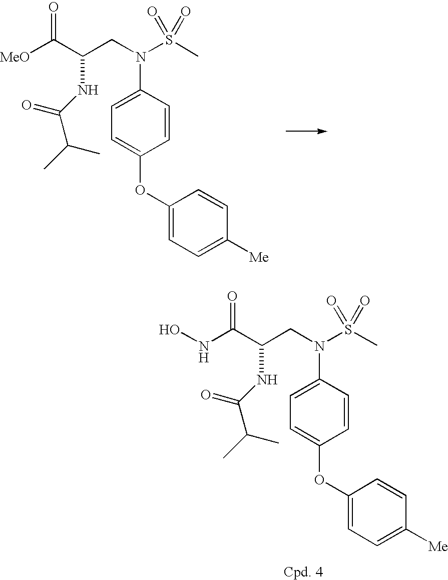

The compound, Methyl (2S)-3-{N-[4-(4-methylphenoxy)phenyl],N-methanesulfonylamino}-2-(2-propylcarbonylamino)propionate, was prepared from 4-(4-methylphenoxy)aniline in accordance with the sequence of general procedure I, II, III, IV and VIII (GP-I, GP-II, GP-III, GP-IV, and GP-VIII) as described herein above for reference examples.

To a mixture of Methyl (2S)-3-{N-[4-(4-methylphenoxy)phenyl],N-methanesulfonylamino}-2-(2-propylcarbonylamino)propionate (112 mg, 0.25 mmol) and hydroxylamine hydrochloride (69.5 mg, 1.0 mmol) in dried MeOH (1 mL) under N₂ at room temperature was added sodium methoxide (0.5 M in MeOH, 3.0 mL, 1.5 mmol). The mixture was stirred at room temperature for 2 h and was poured into EtOAc/H₂O (100 mL/100 mL) and HCl_{(aq)} (2.0 N, 2.0 mL) was added. The aqueous layer was extracted with EtOAc (50 mL). The combined organic layers was dried (Na₂SO₄) and filtered. After removal of solvent, the product was resolidified from CH₂Cl₂/Et₂O/hexane (1/2/7) to give 100 mg of N-Hydroxy (2S)-3-{N-[4-(4-methylphenoxy)phenyl],N-methanesulfonylamino}-2-(2-propylcarbonylamino)propionamide (89%) as a white solid. ¹H NMR (300 MHz, d₆-DMSO) δ 10.70 (s, 1H), 8.89 (s, 1H), 7.80 (d, J=9.0 Hz, 1H), 7.31 (d, J=9.0 Hz, 2H), 7.22 (d, J=9.0 Hz, 2H), 6.98-6.93 (m, 4H), 4.20 (dd, J=15.0, 6.0 Hz, 1H), 3.86-3.72 (m, 2H), 2.97 (s, 3H), 2.40 (quint, J=6.0 Hz, 1H), 2.30 (s, 3H), 0.99 (d, J=6.0 Hz, 3H), 0.94 (d, J=6.0 Hz, 3H); MS (EI, m/z): 472 (M⁺+Na, 100), 450 (M⁺+1). Anal. Calcd for C₂₁H₂₇N₃O₆S·0.5H₂O: C, 55.01; H, 615; N, 9.16. Found: C, 54.53; H, 5.98; N, 8.80.

### Example 5

### N-Hydroxy,N-(2-methylpropyl)(2S)-2-(morpholin-4-yl)-3-{N-[4-(4-trifluoromethylphenoxy)phenyl],N-methanesulfonylamino}propionamide

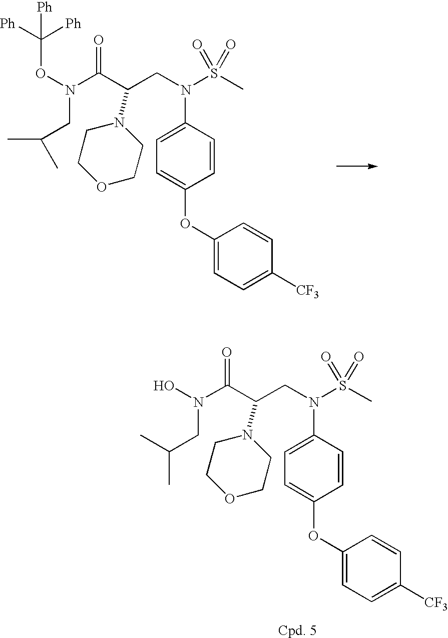

The compound, N-(2-Methylpropyl),N-triphenylmethoxy(2S)-2-(morpholin-4-yl)-3-{N-[4-(4-trifluoromethylphenoxy)phenyl],N-methanesulfonylamino}propionamide, was prepared from 4-(4-trifluoromethylphenoxy)aniline in accordance with the sequence of general procedure I, II, III, IV, V, VI, and IX (GP-I, GP-II, GP-VIII, GP-IV, GP-V, GP-VI, and GP-IX) as described herein above for reference examples.

To a solution of N-(2-Methylpropyl),N-triphenylmethoxy(2S)-2-(morpholin-4-yl)-3-{N-[4-(4-trifluoromethylphenoxy)phenyl],N-methanesulfonylamino}propionamide (80 mg, 0.1 mmol) in Et₂O (0.2 mL) was added trifluoroacetic acid (1 mL) at room temperature. The mixture was stirred at rt for 1 h. Pure H₂O (1 mL) and Et₂O (1 mL) were added sequentially. The mixture was poured into Et₂O/H₂O (20 mL/50 mL). Hexane (30 mL) was added into the extraction funnel. The organic layer was extracted with pure H₂O (50 mL×3). The combined aqueous layers was lyophilized to give 50 mg of N-Hydroxy,N-(2-methylpropyl)(2S)-2-(morpholin-4-yl)-3-{N-[4-(4-trifluoromethylphenoxy)phenyl],N-methanesulfonylamino}propionamide (75%) as a white trifluoroacetic acid salt. ¹H NMR (300 MHz, d₆-DMSO) δ 10.20 (br s, 1H), 7.77 (d, J=6.0 Hz, 2H), 7.46 (d, J=9.0 Hz, 2H), 7.19 (d, J=9.0 Hz, 2H), 7.18 (d, J=9.0 Hz, 2H), 4.20-4.10 (m, 1H), 3.95-3.85 (m, 1H), 3.70-3.50 (m, 6H), 3.03 (s, 3H), 2.85-2.60 (m, 4H), 1.77 (quint, J=6.0 Hz, 1H), 0.86-0.77 (m, 6H); MS (EI, m/z): 582 (M⁺+Na), 560 (M⁺+1, 100). Anal. Calcd for C₂₅H₃₂F₃N₃O₆S·1.0 TFA: C, 48.14; H, 4.94; N, 6.24. Found: C, 47.96; H, 4.66; N, 6.12.

### Example 6

### N-Hydroxy 2-hydroxy-2-methyl-3-{N-[4-(4-methylphenoxy)phenyl],N-methanesulfonylamino}propionamide

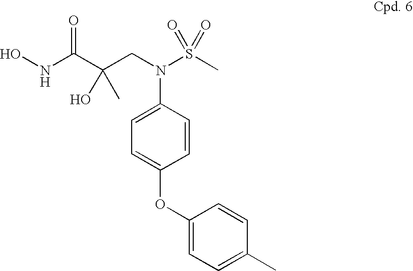

The title compound was prepared form the 4-(4-methylphenoxy)aniline in a similar manner as described herein above for Example 1. MS (EI, m/z): 417 (M⁺+Na), 395 (M⁺+1).

### Example 7

### N-Hydroxy (2R)-2-hydroxy-3-{N-[4-(4-methylphenoxy)phenyl],N-methanesulfonylamino}propionamide

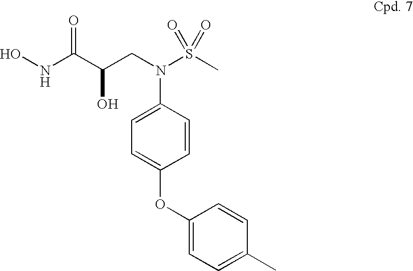

The title compound was prepared form the 4-(4-methylphenoxy)aniline in a similar manner as described herein above for Example 1. MS (EI, m/z): 379 (M⁺−1, 100).

### Example 8

### N-Hydroxy (2S)-2-hydroxy-3-{N-[4-(4-methylphenoxy)phenyl],N-methanesulfonylamino}propionamide

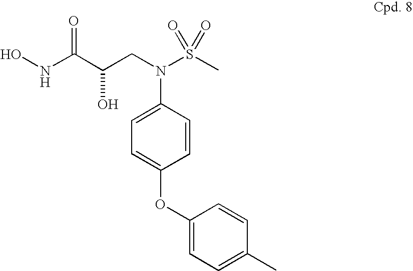

The title compound was prepared form the 4-(4-methylphenoxy)aniline in a similar manner as described herein above for Example 1. MS (EI, m/z): 403 (M⁺+Na, 100).

### Example 9

### N-Hydroxy (2R)-2-hydroxy-3-[N-(4-phenyl)phenyl,N-methanesulfonylamino]propionamide

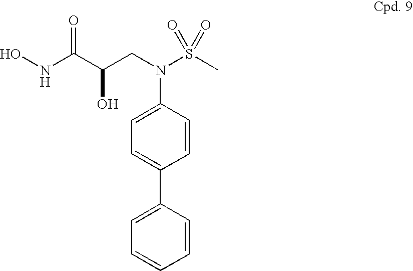

The title compound was prepared form the 4-phenylaniline in a similar manner as described herein above for Example 1. MS (EI, m/z): 349 (M⁺−1, 100).

### Example 10

### N-Hydroxy (2S)-2-hydroxy-3-[N-(4-phenyl)phenyl,N-methanesulfonylamino]propionamide

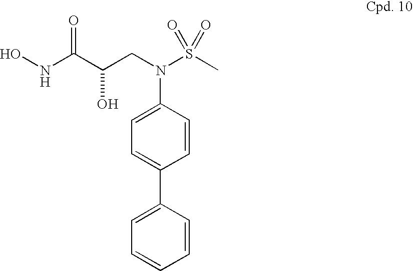

The title compound was prepared form the 4-phenylaniline in a similar manner as described herein above for Example 1. MS (EI, m/z): 373 (M⁺+Na, 100), 351 (M⁺+1).

### Example 11

### N-Hydroxy (2R)-2-hydroxy-3-[N-(4-phenoxy)phenyl,N-methanesulfonylamino]propionamide

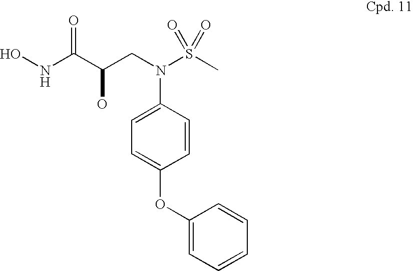

The title compound was prepared form the 4-phenoxyaniline in a similar manner as described herein above for Example 1. MS (EI, m/z): 365 (M⁺−1, 100).

### Example 12

### N-Hydroxy (2S)-2-hydroxy-3-[N-(4-phenoxy)phenyl,N-methanesulfonylamino]propionamide

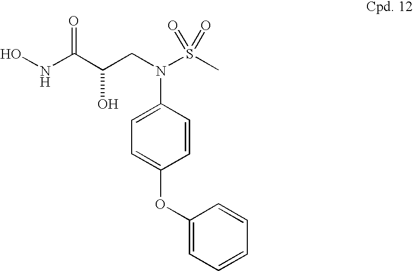

The title compound was prepared form the 4-phenoxyaniline in a similar manner as described herein above for Example 1. MS (EI, m/z): 365 (M⁺−1, 100).

### Example 13

### N-Hydroxy (2R)-2-hydroxy-3-{N-[4-(4-chlorophenoxy)phenyl],N-methanesulfonylamino}propionamide

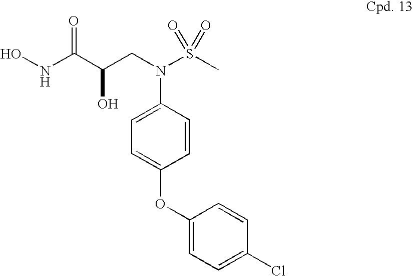

The title compound was prepared form the 4-(4-chlorophenoxy)aniline in a similar manner as described herein above for Example 1. MS (EI, m/z): 423 (M⁺+Na, 100), 401 (M⁺+1).

### Example 14

### N-Hydroxy (2S)-2-hydroxy-3-{N-[4-(4-methylphenoxy)phenyl],N-phenylmethylsulfonylamino}propionamide

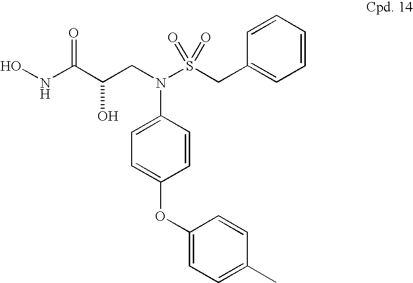

The title compound was prepared form the 4-(4-methylphenoxy)aniline in a similar manner as described herein above for Example 1. MS (EI, m/z): 455 (M⁺−1, 100).

### Example 15

### N-Hydroxy (2S)-2-hydroxy-3-{N-[4-(4-methylphenoxy)phenyl],N-(1-propyl)sulfonylamino}propionamide

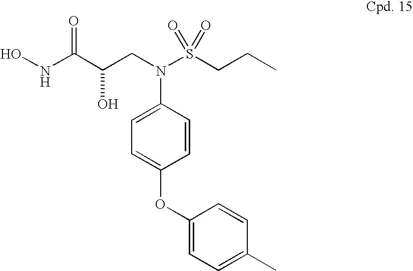

The title compound was prepared form the 4-(4-methylphenoxy)aniline in a similar manner as described herein above for Example 1. MS (EI, m/z): 407 (M⁺−1, 100).

### Example 16

### N-Hydroxy (2S)-2-hydroxy-3-{N-[4-(4-methylphenoxy)phenyl],N-(2-propyl)sulfonylamino}propionamide

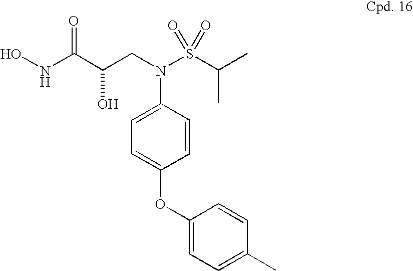

The title compound was prepared form the 4-(4-methylphenoxy)aniline in a similar manner as described herein above for Example 1. MS (EI, m/z): 407 (M⁺−1, 100).

### Example 17

### N-Hydroxy (2R)-2-methoxy-3-{N-[4-(4-methylphenoxy)phenyl],N-methanesulfonylamino}propionamide

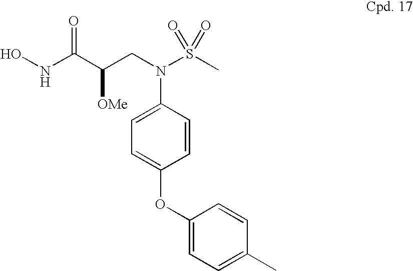

The title compound was prepared form the 4-(4-methylphenoxy)aniline in a similar manner as described herein above for Example 2. MS (EI, m/z): 417 (M⁺+Na), 395 (M⁺+1).

### Example 18

### N-Hydroxy (2S)-3-{N-[4-(4-methylphenoxy)phenyl],N-methanesulfonylamino}-2-[(2-propyl)amino]propionamide

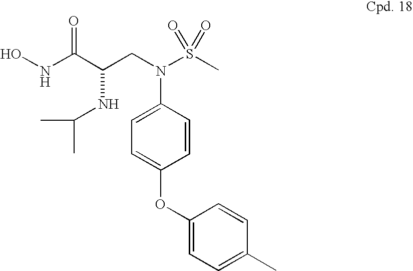

The title compound was prepared form the 4-(4-methylphenoxy)aniline in a similar manner as described herein above for Example 3. MS (EI, m/z): 444 (M⁺+Na), 422 (M⁺+1, 100).

### Example 19

### N-Hydroxy (2S)-2-hydroxy-3-{N-[4-(4-methylphenoxy)phenyl],N-(dimethylamino)sulfonylamino}propionamide

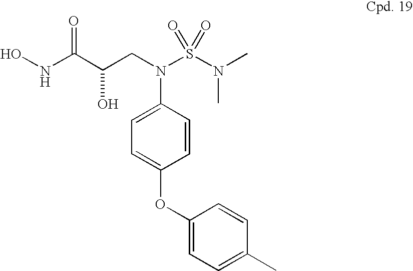

The title compound was prepared form the 4-(4-methylphenoxy)aniline in a similar manner as described herein above for Example 1. MS (EI, m/z): 432 (M⁺+Na), 410 (M⁺+1, 100).

### Example 20

### N-Hydroxy (2S)-2-hydroxy-3-{N-[4-(4-methylphenoxy)phenyl],N-ethylsulfonylamino}propionamide

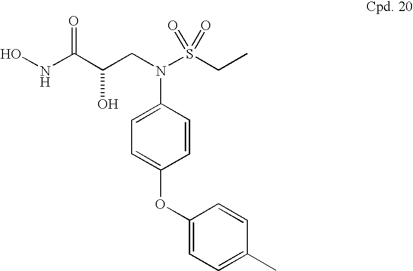

The title compound was prepared form the 4-(4-methylphenoxy)aniline in a similar manner as described herein above for Example 1. MS (EI, m/z): 417 (M⁺+Na, 100).

### Example 21

### N-Hydroxy (2S)-2-hydroxy-3-{N-[4-(4-methylphenoxy)phenyl],N-phenylsulfonylamino}propionamide

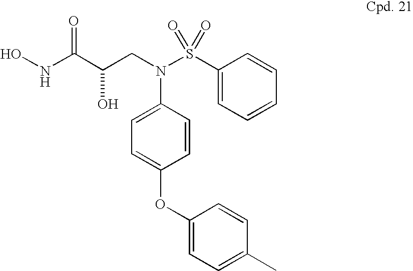

The title compound was prepared form the 4-(4-methylphenoxy)aniline in a similar manner as described herein above for Example 1. MS (EI, m/z): 465 (M⁺+Na), 443 (M⁺+1).

### Example 22

### N-Hydroxy (2R)-2-benzyloxy-3-{N-[4-(4-methylphenoxy)phenyl],N-methanesulfonylamino}propionamide

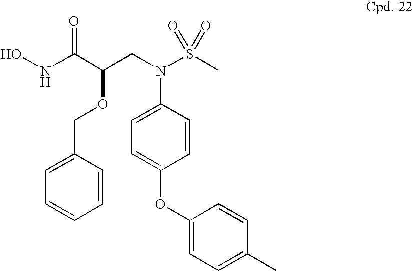

The title compound was prepared form the 4-(4-methylphenoxy)aniline in a similar manner as described herein above for Example 2. MS (EI, m/z): 469 (M⁺−1, 100).

### Example 23

### N-Hydroxy (2S)-2-benzyloxy-3-{N-[4-(4-methylphenoxy)phenyl],N-methanesulfonylamino}propionamide

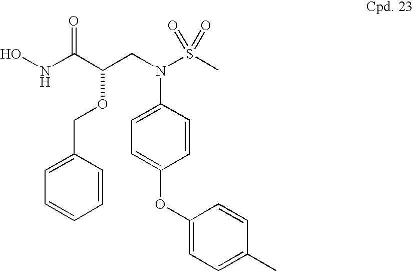

The title compound was prepared form the 4-(4-methylphenoxy)aniline in a similar manner as described herein above for Example 2. MS (EI, m/z): 469 (M⁺−1, 100).

### Example 24

### N-Hydroxy (2R)-3-{N-[4-(4-methylphenoxy)phenyl],N-methanesulfonylamino}-2-(2-propyl)aminopropionamide

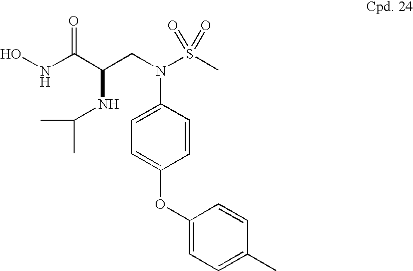

The title compound was prepared form the 4-(4-methylphenoxy)aniline in a similar manner as described herein above for Example 3. ¹H NMR (400 MHz, d₆-DMSO) δ 10.50 (br s, 1H), 8.85 (br s, 1H), 7.36 (d, J=8.0 Hz, 2H), 7.23 (d, J=8.0 Hz, 2H), 6.97 (d, J=8.0 Hz, 2H), 6.96 (d, J=8.0, 2H), 3.71-3.51 (m, 2H), 3.10-2.95 (m, 1H), 3.02 (s, 3H), 2.59 (quint, J=4.0 Hz, 1H), 2.31 (s, 3H), 0.91 (d, J=4.0 Hz, 3H), 0.86 (d, J=4.0 Hz, 3H); MS (EI, m/z): 444 (M⁺+Na), 422 (M⁺+1, 100).

### Example 25

### N-Hydroxy (2S)-2-benzylamino-3-{N-[4-(4-methylphenoxy)phenyl],N-methanesulfonylamino}propionamide

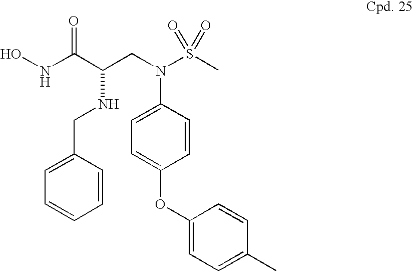

The title compound was prepared form the 4-(4-methylphenoxy)aniline in a similar manner as described herein above for Example 3. MS (EI, m/z): 492 (M⁺+Na), 470 (M⁺+1, 100).

### Example 26

### N-Hydroxy (2R)-2-benzylamino-3-{N-[4-(4-methylphenoxy)phenyl],N-methanesulfonylamino}propionamide

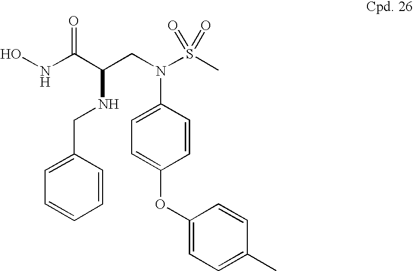

The title compound was prepared form the 4-(4-methylphenoxy)aniline in a similar manner as described herein above for Example 3. MS (EI, m/z): 492 (M⁺+Na), 470 (M⁺+1, 100).

### Example 27

### N-Hydroxy (2S)-3-{N-[4-(4-methylphenoxy)phenyl],N-methanesulfonylamino}-2-(morpholin-4-yl)propionamide

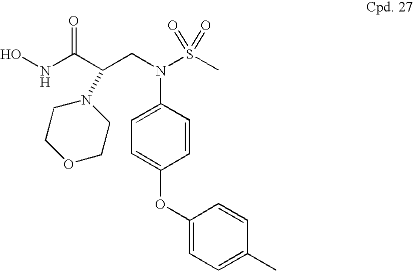

The title compound was prepared form the 4-(4-methylphenoxy)aniline in a similar manner as described herein above for Example 3. MS (EI, m/z): 472 (M⁺+Na), 450 (M⁺+1, 100).

### Example 28

### N-Hydroxy (2R)-3-{N-[4-(4-methylphenoxy)phenyl],N-methanesulfonylamino}-2-(morpholin-4-yl)propionamide

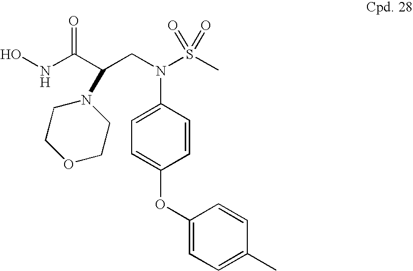

The title compound was prepared form the 4-(4-methylphenoxy)aniline in a similar manner as described herein above for Example 3. MS (EI, m/z): 450 (M⁺+1, 100).

### Example 29

### N-Hydroxy (2S)-2-amino-3-{N-[4-(4-methylphenoxy)phenyl],N-methanesulfonylamino}propionamide

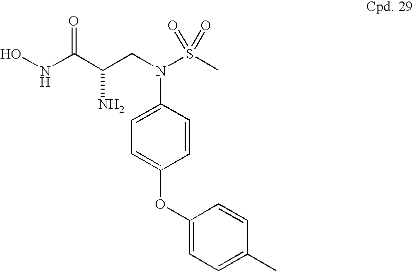

The title compound was prepared form the 4-(4-methylphenoxy)aniline in a similar manner as described herein above for Example 3. ¹H NMR (300 MHz, d₆-DMSO) δ 7.39 (d, J=9.0 Hz, 2H), 7.22 (d, J=9.0 Hz, 2H), 6.99-6.95 (m, 4H), 3.63-3.30 (m, 3H), 3.00 (s, 3H), 2.31 (s, 3H); MS (EI, m/z): 378 (M⁺−1, 100).

### Example 30

### N-Hydroxy (2S)-3-{N-[4-(4-methylphenoxy)phenyl],N-methanesulfonylamino}-2-phenylaminopropionamide

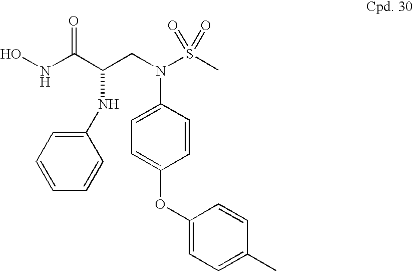

The title compound was prepared form the 4-(4-methylphenoxy)aniline in a similar manner as described herein above for Example 3. MS (EI, m/z): 478 (M⁺+Na).

### Example 31

### N-Hydroxy (2S)-2-hydroxy-3-{N-[4-(4-methylphenoxy)phenyl],N-(pryidin-4-yl)methylsulfonylamino}propionamide

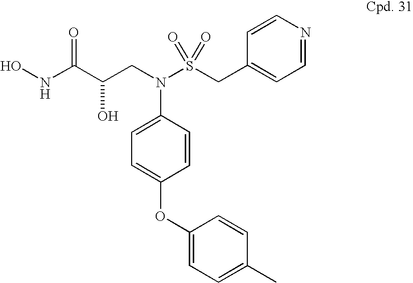

The title compound was prepared form the 4-(4-methylphenoxy)aniline in a similar manner as described herein above for Example 1. MS (EI, m/z): 458 (M⁺+1, 100).

### Example 32

### N-Hydroxy (2S)-2-hydroxy-3-{N-[4-(4-methylphenoxy)phenyl],N-(pryidin-3-yl)methylsulfonylamino}propionamide

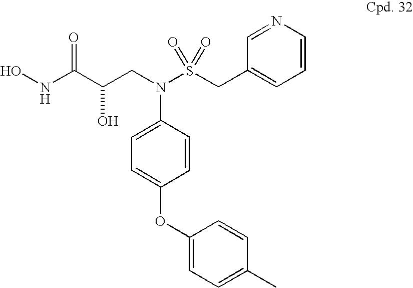

The title compound was prepared form the 4-(4-methylphenoxy)aniline in a similar manner as described herein above for Example 1. MS (EI, m/z): 480 (M⁺+Na), 458 (M⁺+1, 100).

### Example 33

### N-Hydroxy (2S)-3-{N-[4-(4-methylphenoxy)phenyl],N-methanesulfonylamino}-2-(pyridine-4-yl)methylaminopropionamide

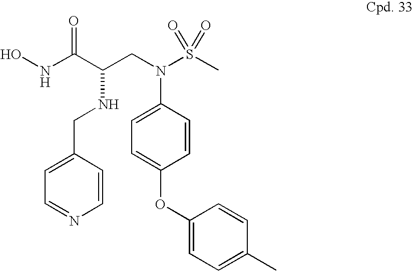

The title compound was prepared form the 4-(4-methylphenoxy)aniline in a similar manner as described herein above for Example 3. MS (EI, m/z): 493 (M⁺+Na), 471 (M⁺+1).

### Example 34

### N-Hydroxy (2S)-3-{N-[4-(4-methylphenoxy)phenyl],N-methanesulfonylamino}-2-(pyridin2-yl)methylaminopropionamide

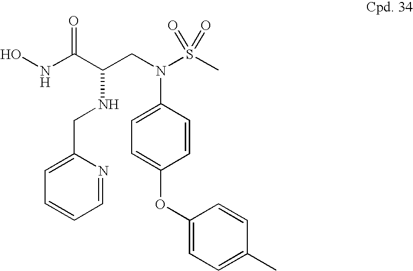

The title compound was prepared form the 4-(4-methylphenoxy)aniline in a similar manner as described herein above for Example 3. MS (EI, m/z): 493 (M⁺+Na), 471 (M⁺+1, 100).

### Example 35

### N-Hydroxy (2S)-3-{N-[4-(4-methylphenoxy)phenyl],N-methanesulfonylamino}-2-(pyridine-3-yl)methylaminopropionamide

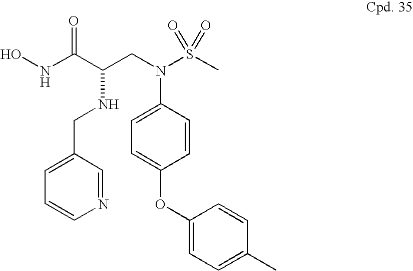

The title compound was prepared form the 4-(4-methylphenoxy)aniline in a similar manner as described herein above for Example 3. ¹H NMR (300 MHz, d₆-DMSO) δ 10.60 (br s, 1H), 8.90 (br s, 1H), 8.43 (m, 2H), 7.67 (d, J=6.0 Hz, 1H), 7.33-7.22 (m, 5H), 6.96 (d, J=9.0 Hz, 2H), 6.90 (d, J=9.0 Hz, 2H), 3.80-3.60 (m, 3H), 3.50-3.40 (m, 1H), 2.99 (s, 3H), 2.99-2.90 (m, 1H), 2.31 (s, 3H); MS (EI, m/z): 471 (M⁺+1, 100).

### Example 36

### N-Hydroxy (2S)-3-{N-[4-(4-methylphenoxy)phenyl],N-methanesulfonylamino}-2-(2-methylpropyl)aminopropionamide

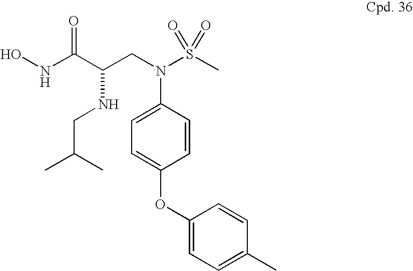

The title compound was prepared form the 4-(4-methylphenoxy)aniline in a similar manner as described herein above for Example 3. MS (EI, m/z): 458 (M⁺+Na), 436 (M⁺+1, 100).

### Example 37

### N-Hydroxy (2S)-2-cyclopentylamino-3-{N-[4-(4-methylphenoxy)phenyl],N-methanesulfonylamino}propionamide

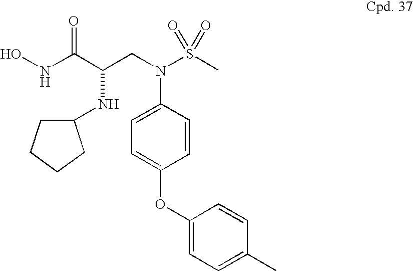

The title compound was prepared form the 4-(4-methylphenoxy)aniline in a similar manner as described herein above for Example 3. MS (EI, m/z): 448 (M⁺+1, 100).

### Example 38

### N-Hydroxy (2S)-2-(4-fluorophenyl)amino-3-{N-[4-(4-methylphenoxy)phenyl],N-methanesulfonylamino}propionamide

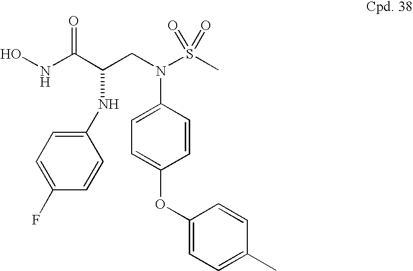

The title compound was prepared form the 4-(4-methylphenoxy)aniline in a similar manner as described herein above for Example 3. MS (EI, m/z): 496 (M⁺+Na), 474 (M⁺+1, 100).

### Example 39

### N-Hydroxy (2S)-2-(3-fluorophenyl)amino-3-{N-[4-(4-methylphenoxy)phenyl],N-methanesulfonylamino}propionamide

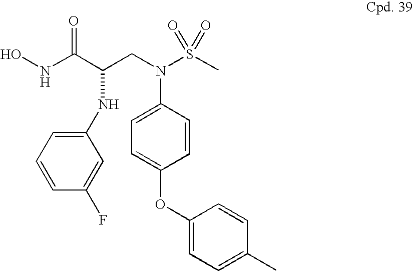

The title compound was prepared form the 4-(4-methylphenoxy)aniline in a similar manner as described herein above for Example 3. ¹H NMR (300 MHz, d₆-DMSO) δ 10.80 (br s, 1H), 8.85 (br s, 1H), 7.29 (d, J=9.0 Hz, 2H), 7.22 (d, J=9.0 Hz, 2H), 7.03-6.92 (m, 5H), 6.42-6.30 (m, 3H), 6.00 (d, J=9.0 Hz, 1H), 3.82-3.74 (m, 3H), 3.01 (s, 3H), 2.31 (s, 3H); MS (EI, m/z): 496 (M⁺+Na, 100), 474 (M⁺+1). Anal. Calcd for C₂₃H₂₄FN₃O₅S·1.0 Na: C, 56.57; H, 4.95; N, 8.25. Found: C, 56.62; H, 5.04; N, 8.34.

### Example 40

### N-Hydroxy (2S)-2-(indol-5-yl)amino-3-{N-[4-(4-methylphenoxy)phenyl],N-methanesulfonylamino}propionamide

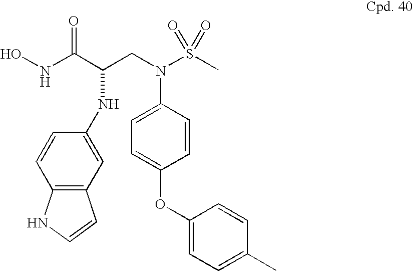

The title compound was prepared form the 4-(4-methylphenoxy)aniline in a similar manner as described herein above for Example 3. MS (EI, m/z): 495 (M⁺+1, 100).

### Example 41

### N-Hydroxy (2S)-2-(3,4-difluorophenyl)amino-3-{N-[4-(4-methylphenoxy)phenyl],N-methanesulfonylamino}propionamide

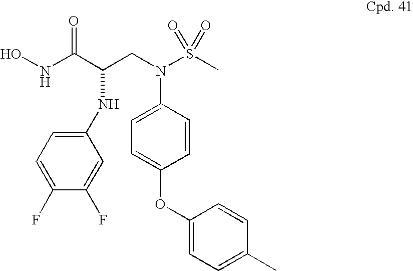

The title compound was prepared form the 4-(4-methylphenoxy)aniline in a similar manner as described herein above for Example 3. MS (EI, m/z): 490 (M⁺−1, 100).

### Example 42

### N-Hydroxy (2S)-2-(3-chloro-4-fluorophenyl)amino-3-{N-[4-(4-methylphenoxy)phenyl],N-methanesulfonylamino}propionamide

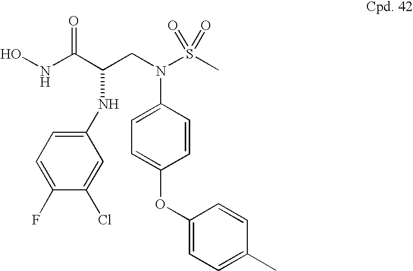

The title compound was prepared form the 4-(4-methylphenoxy)aniline in a similar manner as described herein above for Example 3. MS (EI, m/z): 530 (M⁺+Na), 508 (M⁺+1, 100).

### Example 43

### N-Hydroxy (2S)-2-(4-chlorophenyl)amino-3-{N-[4-(4-methylphenoxy)phenyl],N-methanesulfonylamino}propionamide

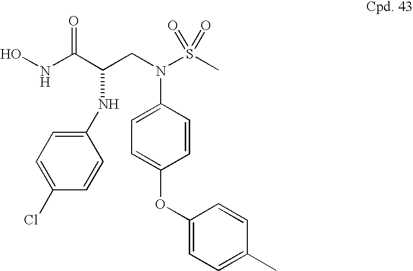

The title compound was prepared form the 4-(4-methylphenoxy)aniline in a similar manner as described herein above for Example 3. MS (EI, m/z): 512 (M⁺+Na), 590 (M⁺+1, 100).

### Example 44

### N-Hydroxy (2S)-2-(3-chlorophenyl)amino-3-{N-[4-(4-methylphenoxy)phenyl],N-methanesulfonylamino}propionamide

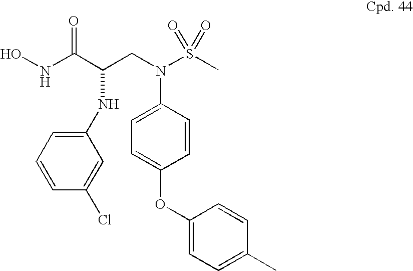

The title compound was prepared form the 4-(4-methylphenoxy)aniline in a similar manner as described herein above for Example 3. MS (EI, m/z): 512 (M⁺+Na), 590 (M⁺+1, 100).

### Example 45

### N-Hydroxy (2S)-3-{N-[4-(4-methylphenoxy)phenyl],N-methanesulfonylamino}-2-(3-trifluorormethylphenyl)aminopropionamide

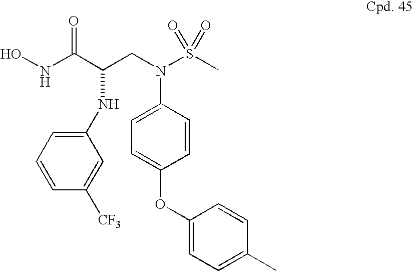

The title compound was prepared form the 4-(4-methylphenoxy)aniline in a similar manner as described herein above for Example 3. MS (EI, m/z): 546 (M⁺+Na), 524 (M⁺+1, 100).

### Example 46

### N-Hydroxy (2S)-3-{N-[4-(4-methylphenoxy)phenyl],N-methanesulfonylamino}-2-[(1R)-1-phenyl-1-ethyl]aminopropionamide

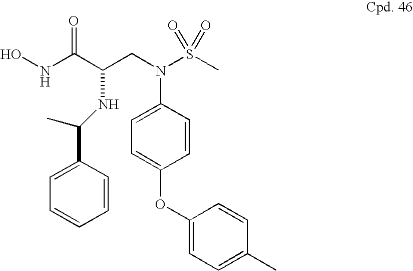

The title compound was prepared form the 4-(4-methylphenoxy)aniline in a similar manner as described herein above for Example 3. MS (EI, m/z): 506 (M⁺+Na), 484 (M⁺+1, 100).

### Example 47

### N-Hydroxy (2S)-3-{N-[4-(4-methylphenoxy)phenyl],N-methanesulfonylamino}-2-[(1S)-1-phenyl-1-ethyl]aminopropionamide

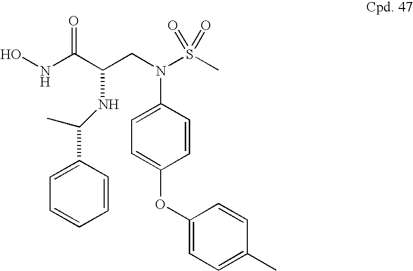

The title compound was prepared form the 4-(4-methylphenoxy)aniline in a similar manner as described herein above for Example 3. MS (EI, m/z): 506 (M⁺+Na), 484 (M⁺+1, 100).

### Example 48

### N-Hydroxy (2S)-2-(4-methoxyphenyl)amino-3-{N-[4-(4-methylphenoxy)phenyl],N-methanesulfonylamino}propionamide

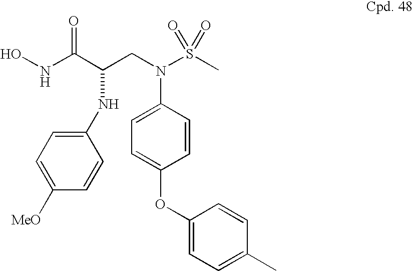

The title compound was prepared form the 4-(4-methylphenoxy)aniline in a similar manner as described herein above for Example 3. MS (EI, m/z): 508 (M⁺+Na, 100), 486 (M⁺+1).

### Example 49

### N-Hydroxy (2S)-2-hydroxy-3-{N-[4-(4-methylthiophenoxy)phenyl],N-methanesulfonylamino}propionamide

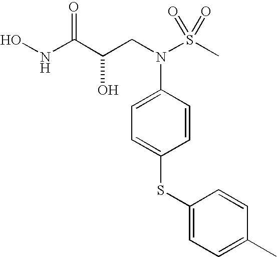

The title compound was prepared form the 4-(4-methylthiophenoxy)aniline in a similar manner as described herein above for Example 1. MS (EI, m/z): 419 (M⁺+Na, 100), 397 (M⁺+1).

### Example 50

### N-Hydroxy (2S)-3-{N-[4-(4-methylphenoxy)phenyl],N-methanesulfonylamino}-2-(4-trifluoromethoxyphenyl)aminopropionamide

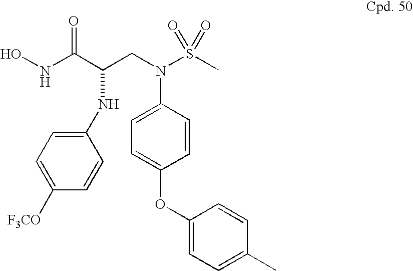

The title compound was prepared form the 4-(4-methylphenoxy)aniline in a similar manner as described herein above for Example 3. MS (EI, m/z): 561 (M⁺+Na), 540 (M⁺+1, 100).

### Example 51

### N-Hydroxy (2S)-2-(3-methoxyphenyl)amino-3-{N-[4-(4-methylphenoxy)phenyl],N-methanesulfonylamino}propionamide

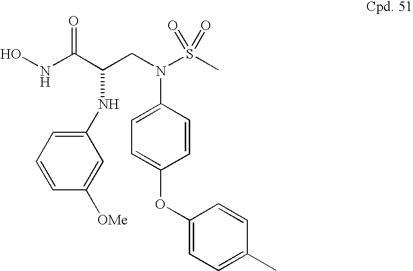

The title compound was prepared form the 4-(4-methylphenoxy)aniline in a similar manner as described herein above for Example 3. MS (EI, m/z): 508 (M⁺+Na), 486 (M⁺+1, 100).

### Example 52

### N-Hydroxy (2S)-3-{N-[4-(4-methylphenoxy)phenyl],N-methanesulfonylamino}-2-(3-trifluoromethoxyphenyl)aminopropionamide

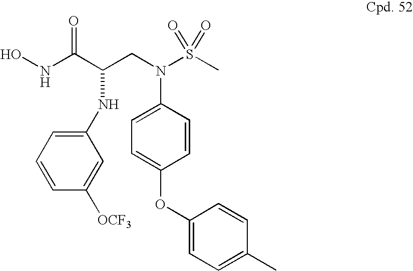

The title compound was prepared form the 4-(4-methylphenoxy)aniline in a similar manner as described herein above for Example 3. MS (EI, m/z): 540 (M⁺+1, 100).

### Example 53

### N-Hydroxy (2S)-2-hydroxy-3-{N-{4-[(5-trifluoromethylpyridin-2-yl)oxy]phenyl},N-methanesulfonylamino}propionamide

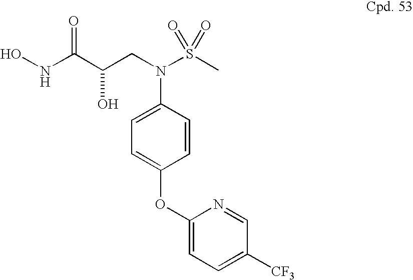

The title compound was prepared form the 4-[(5-trifluoromethylpyridin-2-yl)oxy]aniline in a similar manner as described herein above for Example 1. MS (EI, m/z): 458 (M⁺+Na, 100), 436 (M⁺+1).

### Example 54

### N-Hydroxy (2S)-2-hydroxy-3-{N-[4-(4-trifluoromethylphenoxy)phenyl],N-methanesulfonylamino}propionamide

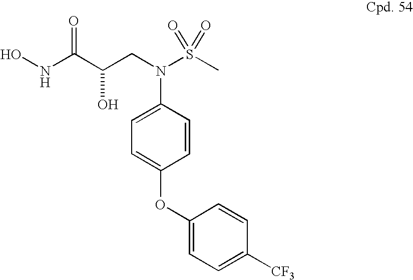

The title compound was prepared form the 4-(4-trifluoromethylphenoxyaniline in a similar manner as described herein above for Example 1. MS (EI, m/z): 457 (M⁺+Na), 435 (M⁺+1).

### Example 55

### N-Hydroxy (2S)-2-(3-fluorophenyl)amino-3-{N-{4-[(5-trifluoromethylpyridin-2-yl)oxy]phenyl},N-methanesulfonylamino}propionamide

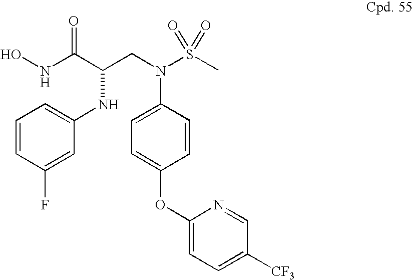

The title compound was prepared form the 4-[(5-trifluoromethylpyridin-2-yl)oxy]aniline in a similar manner as described herein above for Example 3. MS (EI, m/z): 551 (M⁺+Na), 529 (M⁺+1, 100).

### Example 56

### N-Hydroxy (2S)-2-(2-propyl)amino-3-{N-{4-[(5-trifluoromethylpyridin-2-yl)oxyl]phenyl},N-methanesulfonylamino}propionamide

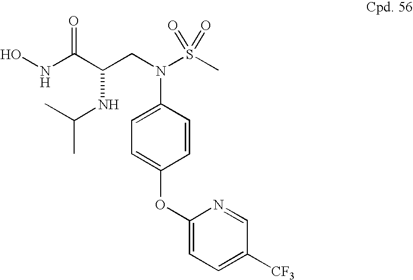

The title compound was prepared form the 4-[(5-trifluoromethylpyridin-2-yl)oxy]aniline in a similar manner as described herein above for Example 3. MS (EI, m/z): 499 (M⁺+Na), 477 (M⁺+1, 100).

### Example 57

### N-Hydroxy (2S)-2-(morpholin-4-yl)-3-{N-{4-[(5-trifluoromethylpyridin-2-yl)oxy]phenyl},N-methanesulfonylamino}propionamide

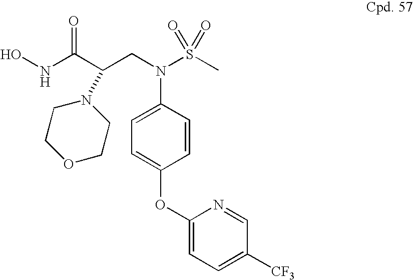

The title compound was prepared form the 4-[(5-trifluoromethylpyridin-2-yl)oxy]aniline in a similar manner as described herein above for Example 3. MS (EI, m/z): 527 (M⁺+Na), 505 (M⁺+1, 100).

### Example 58

### N-Hydroxy (2S)-2-(2-propyl)amino-3-{N-[4-(4-trifluoromethylphenoxy)phenyl],N-methanesulfonylamino}propionamide

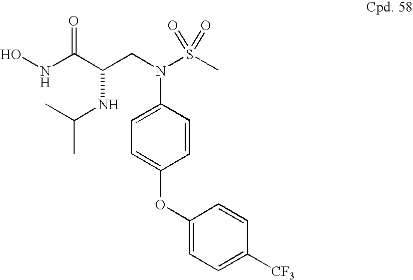

The title compound was prepared form the 4-(4-trifluoromethylphenoxy)aniline in a similar manner as described herein above for Example 3. MS (EI, m/z): 498 (M⁺+Na), 476 (M⁺+1, 100).

### Example 59

### N-Hydroxy (2S)-2-(3-fluorophenyl)amino-3-{N-[4-(4-trifluoromethylphenoxy)phenyl],N-methanesulfonylamino}propionamide

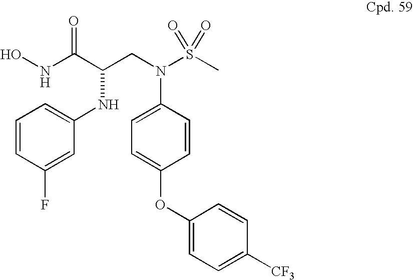

The title compound was prepared form the 4-(4-trifluoromethylphenoxy)aniline in a similar manner as described herein above for Example 3. MS (EI, m/z): 550 (M⁺+Na), 528 (M⁺+1).

### Example 60

### N-Hydroxy (2S)-3-{N-[4-(4-methylphenoxy)phenyl],N-methanesulfonylamino}-2-(naphthylen-2-yl)aminopropionamide

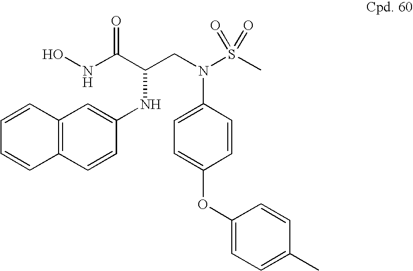

The title compound was prepared form the 4-(4-methylphenoxy)aniline in a similar manner as described herein above for Example 3. MS (EI, m/z): 528 (M⁺+Na), 506 (M⁺+1, 100).

### Example 61

### N-Hydroxy (2S)-2-hydroxy-3-{N-[4-(4-methoxyphenylacetylene)phenyl],N-methanesulfonylamino}propionamide

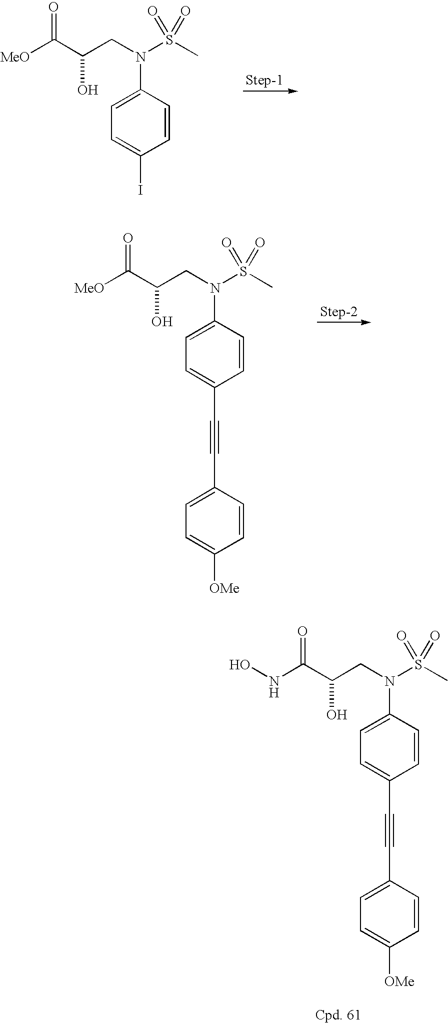

The compound, Methyl (2S)-2-hydroxy-3-[N-(4-iodophenyl),N-methanesulfonylamino]propionate, was prepared from 4-iodoaniline in accordance with the sequence of general procedure I and II (GP-I and GP-II) as described herein above for reference examples.

Step-1:

In a 2-neck flask was placed Methyl (2S)-2-hydroxy-3-[N-(4-iodophenyl),N-methanesulfonylamino]propionate (200 mg, 0.5 mmol), dichloro-bistriphenylphosphinepalladium (35 mg, 0.05 mmol), and CuI (9.5 mg, 0.05 mmol). The air was removed and was refilled with N₂ for three times. Then, THF (1 mL), 4-methoxyphenylacetylene (0.13 mL, 1.0 mmol), and Et₃N (0.14 mL, 1.0 mmol) were added sequentially. The mixture was stirred at room temperature for 2 h and was then poured into EtOAc/H₂O (50 mL/50 mL). The organic layer was dried (Na₂SO₄) and filtered. After removal of solvent, the crude product was purified by silica gel chromatography using EtOAc/hexane (1/4 to 1/1) as the eluent to give 163 mg of Methyl (2S)-2-hydroxy-3-{N-[4-(4-methoxyphenylacetylene)phenyl],N-methanesulfonylamino}propionate (81%) as a pale brown solid. ¹H NMR (400 MHz, CDCl₃) δ 7.54 (d, J=8.0 Hz, 2H), 7.46 (d, J=9.0 Hz, 2H), 7.35 (d, J=8.0 Hz, 2H), 6.89 (d, J=8.0 Hz, 2H), 4.28 (dd, J=12.0, 8.0 Hz, 1H), 4.05 (d, J=8.0 Hz, 2H), 3.84 (s, 3H), 3.66 (s, 3H), 3.06 (d, J=8.0 Hz, 1H), 3.01 (s, 3H); MS (EI, m/z): 426 (M⁺+Na, 100).

Step-2:

To a mixture of Methyl (2S)-2-hydroxy-3-{N-[4-(4-methoxyphenylacetylene)phenyl],N-methanesulfonylamino}propionate (101 mg, 0.25 mmol) and hydroxylamine hydrochloride (69.5 mg, 1.0 mmol) in dried MeOH (1 mL) under N₂ at room temperature was added sodium methoxide (0.5 M in MeOH, 3.0 mL, 1.5 mmol). The mixture was stirred at room temperature for 30 min and was poured into EtOAc/H₂O (50 mL/50 mL) and HCl_{(aq)} (2 N, 3 mL) was added. The organic layer was dried (Na₂SO₄) and filtered. After removal of solvent, the product was dried to give 94 mg of N-Hydroxy (2S)-2-hydroxy-3-{N-[4-(4-methoxyphenylacetylene)phenyl],N-methanesulfonylamino}propionamide (93%) as a white solid. ¹H NMR (300 MHz, d₆-DMSO) δ 10.61 (s, 1H), 8.76 (s, 1 H), 7.56 (d, J=9.0 Hz, 2H), 7.50 (d, J=9.0 Hz, 2H), 7.44 (d, J=9.0 Hz, 2H), 6.99 (d, J=9.0 Hz, 2H), 5.73 (d, J=6.0 Hz, 1H), 3.89-3.71 (m, 3H), 3.80 (s, 3H), 3.06 (s, 3H); MS (EI, m/z): 403 (M⁺−1, 100). Anal. Calcd for C₁₉H₂₀N₂O₆S: C, 56.42; H, 4.98; N, 6.93. Found: C, 56.41; H, 4.88; N, 6.83.

### Example 62

### N-Hydroxy (2S)-2-hydroxy-3-{N-[4-(4-chlorophenylacetylene)phenyl],N-methanesulfonylamino}propionamide

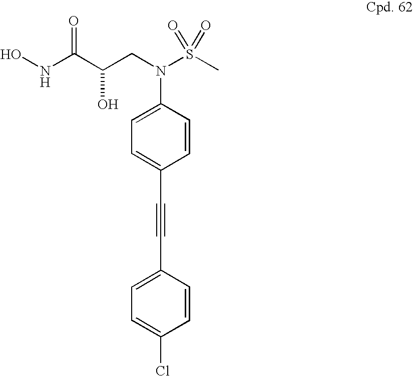

The title compound was prepared from Methyl (2S)-2-hydroxy-3-[N-(4-iodophenyl),N-methanesulfonylamino]propionate in a similar manner as described for Example 61, except using the 4-chlorophenylacetylene as the cross-coupling reagent in Step-1. MS (EI, m/z): 407 (M⁺−1, 100).

### Example 63

### N-Hydroxy (2S)-2-hydroxy-3-{N-[4-(4-methylphenylacetylene)phenyl],N-methanesulfonylamino}propionamide

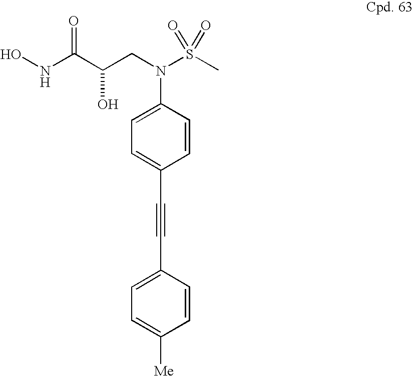

The title compound was prepared from Methyl (2S)-2-hydroxy-3-[N-(4-iodophenyl),N-methanesulfonylamino]propionate in a similar manner as described for Example 61, except using the 4-methylphenylacetylene as the cross-coupling reagent in Step-1. MS (EI, m/z): 387 (M⁺−1, 100).

### Example 64

### N-Hydroxy (2S)-2-hydroxy-3-{N-[4-(4-thiophen-2-ylacetylene)phenyl],N-methanesulfonylamino}propionamide

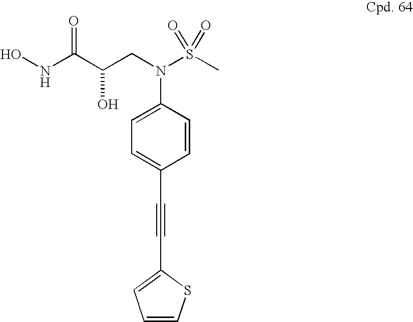

The title compound was prepared from Methyl (2S)-2-hydroxy-3-[N-(4-iodophenyl),N-methanesulfonylamino]propionate in a similar manner as described for Example 61, except using the 2-acetylenethiophene as the cross-coupling reagent in Step-1. MS (EI, m/z): 379 (M⁺−1, 100).

### Example 65

### N-Hydroxy (2S)-2-[4-(4-bromophenyl)-4-hydroxypiperidin-1-yl]-3-{N-[4-(4-methylphenoxy)phenyl],N-methanesulfonylamino}propionamide

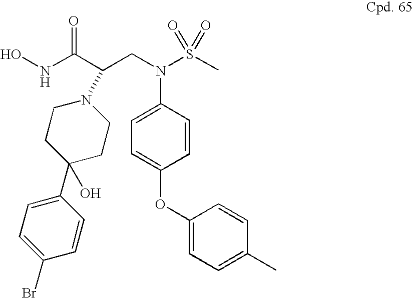

The title compound was prepared form the 4-(4-methylphenoxy)aniline in a similar manner as described herein above for Example 3. MS (EI, m/z): 619 (M⁺+, 100), 617 (M⁺+1).

### Example 66

### N-Hydroxy (2S)-2-[(2-(indol-3-yl)ethylamino]-3-{N-[4-(4-methylphenoxy)phenyl],N-methanesulfonylamino}propionamide

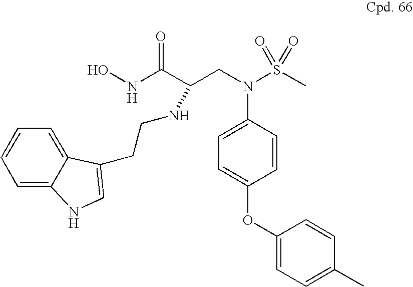

The title compound was prepared form the 4-(4-methylphenoxy)aniline in a similar manner as described herein above for Example 3. MS (EI, m/z): 523 (M⁺+1, 100).

### Example 67

### N-Hydroxy (2S)-2-[(4-benzyloxycarbonyl)piperazin-1-yl]-3-{N-[4-(4-trifluoromethylphenoxy)phenyl],N-methanesulfonylamino}propionamide

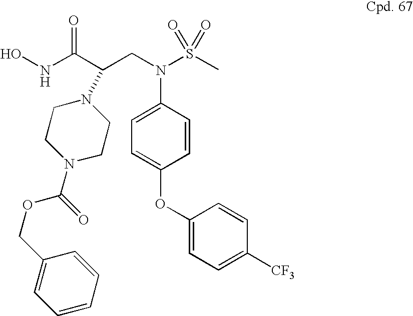

The title compound was prepared form the 4-(4-trifluoremethylphenoxy)aniline in a similar manner as described herein above for Example 3. MS (EI, m/z): 659 (M⁺+Na), 637 (M⁺+1, 100).

### Example 68

### N-Hydroxy (2S)-2-{N-(2-propyl),N-[2-benzyloxycarbonylamino)ethyl]amino}-3-{N-[4-(4-trifluoromethylphenoxy)phenyl],N-methanesulfonylamino}propionamide

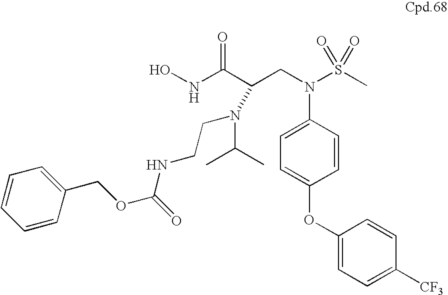

The title compound was prepared form the 4-(4-trifluoremethylphenoxy)aniline in a similar manner as described herein above for Example 3. MS (EI, m/z): 653 (M⁺+1, 100).

### Example 69

### N-Hydroxy (2S)-2-[N-benzyl-N-(2-propyl)amino]-3-{N-[4-(4-trifluoromethylphenoxy)phenyl],N-methanesulfonylamino}propionamide

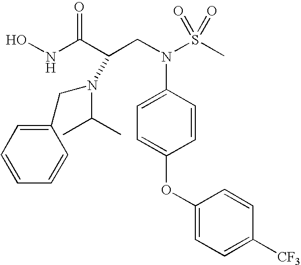

The title compound was prepared form the 4-(4-trifluoremethylphenoxy)aniline in a similar manner as described herein above for Example 3. MS (EI, m/z): 566 (M⁺+1, 100).

### Example 70

### N-Hydroxy (2S)-2-[1,1-bis(oxo)thiomorpholin-4-yl]-3-{N-[4-(4-trifluoromethylphenoxy)phenyl],N-methanesulfonylamino}propionamide

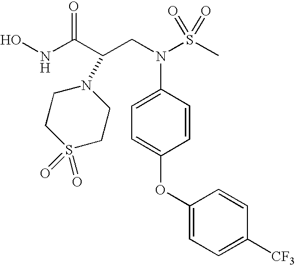

The title compound was prepared form the 4-(4-trifluoremethylphenoxy)aniline in a similar manner as described herein above for Example 3. MS (EI, m/z): 552 (M⁺+1, 100).

### Example 71

### N-Hydroxy (2S)-2-(4-hydroxypiperidin-1-yl)-3-{N-[4-(4-trifluoromethylphenoxy)phenyl],N-methanesulfonylamino}propionamide

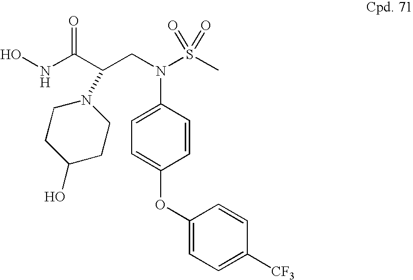

The title compound was prepared form the 4-(4-trifluoremethylphenoxy)aniline in a similar manner as described herein above for Example 3. MS (EI, m/z): 540 (M⁺+Na), 518 (M⁺+1, 100).

### Example 72

### N-Hydroxy (2S)-2-(isoindolin-2-yl)-3-{N-[4-(4-trifluoromethylphenoxy)phenyl],N-methanesulfonylamino}propionamide

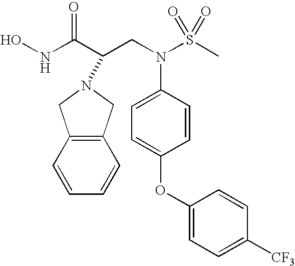

The title compound was prepared form the 4-(4-trifluoremethylphenoxy)aniline in a similar manner as described herein above for Example 3. MS (EI, m/z): 536 (M⁺+1, 100).

### Example 73

### N-Hydroxy (2S)-2-(1,2,3,4-tetrahydroisoquinolin-2-yl)-3-{N-[4-(4-trifluoromethylphenoxy)phenyl],N-methanesulfonylamino}propionamide

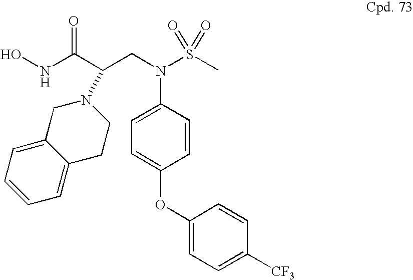

The title compound was prepared form the 4-(4-trifluoremethylphenoxy)aniline in a similar manner as described herein above for Example 3. MS (EI, m/z): 550 (M⁺+1, 100).

### Example 74

### N-Hydroxy (2S)-2-[(3S)-3-N,N-dimethylaminopyrrolidin-1-yl]-3-{N-[4-(4-trifluoromethylphenoxy)phenyl],N-methanesulfonylamino}propionamide

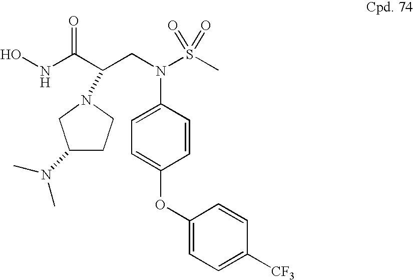

The title compound was prepared form the 4-(4-trifluoremethylphenoxy)aniline in a similar manner as described herein above for Example 3. MS (EI, m/z): 553 (M⁺+Na), 531 (M⁺+1, 100).

### Example 75

### N-Hydroxy (2S)-2-[(3R)-3-N,N-dimethylaminopyrrolidin-1-yl]-3-{N-[4-(4-trifluoromethylphenoxy)phenyl],N-methanesulfonylamino}propionamide

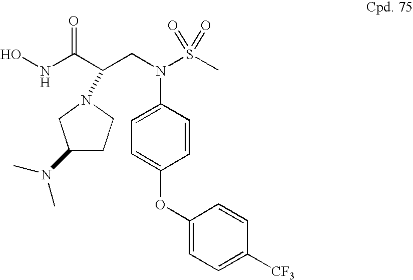

The title compound was prepared form the 4-(4-trifluoremethylphenoxy)aniline in a similar manner as described herein above for Example 3. MS (EI, m/z): 531 (M⁺+1, 100).

### Example 76

### N-Hydroxy (2S)-3-{N-[4-(4-trifluoromethylphenoxy)phenyl],N-methanesulfonylamino}-2-(4-phenylpiperidin-1-yl)propionamide

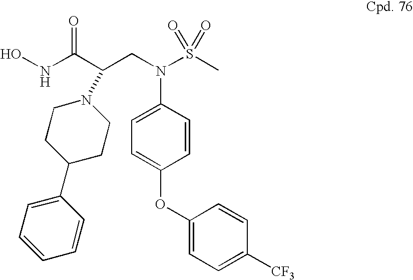

The title compound was prepared form the 4-(4-trifluoremethylphenoxy)aniline in a similar manner as described herein above for Example 3. MS (EI, m/z): 578 (M⁺+1, 100).

### Example 77

### N-Hydroxy (2S)-2-[4-(4-chlorophenyl)piperazin-1-yl]-3-{N-[4-(4-trifluoromethylphenoxy)phenyl],N-methanesulfonylamino}propionamide

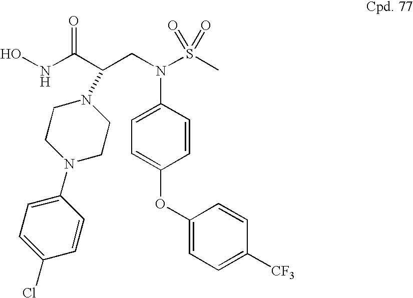

The title compound was prepared form the 4-(4-trifluoremethylphenoxy)aniline in a similar manner as described herein above for Example 3. MS (EI, m/z): 613 (M⁺+1, 100).

### Example 78

### N-Hydroxy (2S)-2-[(2-(indol-3-yl)ethylamino]-3-{N-[4-(4-trifluoromethylphenoxy)phenyl],N-methanesulfonylamino}propionamide

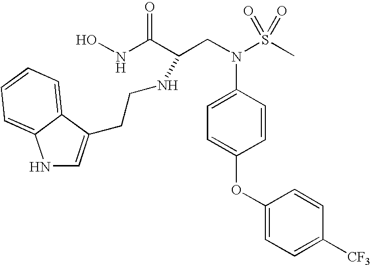

The title compound was prepared form the 4-(4-trifluoremethylphenoxy)aniline in a similar manner as described herein above for Example 3. MS (EI, m/z): 577 (M⁺+1).

### Example 79

### N-Hydroxy (2S)-2-[4-(4-bromophenyl)-4-hydroxypiperidin-1-yl]-3-{N-[4-(4-trifluoromethylphenoxy)phenyl],N-methanesulfonylamino}propionamide

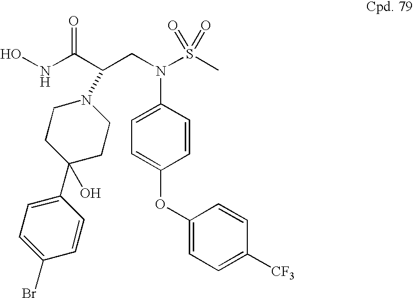

The title compound was prepared form the 4-(4-trifluoremethylphenoxy)aniline in a similar manner as described herein above for Example 3. MS (EI, m/z): 674 (M⁺+1, 100), 672 (M⁺+1).

### Example 80

### N-Hydroxy (2S)-2-{N-(2-propyl),N-[2-(1,5,5-trimethylhydantoin-3-yl)ethyl]amino}-3-{N-[4-(4-trifluoromethylphenoxy)phenyl]-N-methanesulfonylamino}propionamide

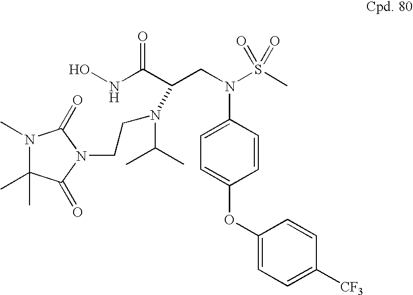

The title compound was prepared form the 4-(4-trifluoremethylphenoxy)aniline in a similar manner as described herein above for Example 3. MS (EI, m/z): 666 (M⁺+Na), 644 (M⁺+1, 100).

### Example 81

### N-Hydroxy (2S)-2-[(2R)-2-methoxymethylpyrrolidin-1-yl]-3-{N-[4-(4-trifluoromethylphenoxy)phenyl],N-methanesulfonylamino}propionamide

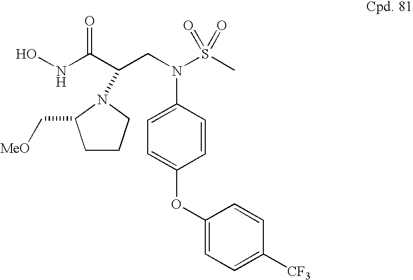

The title compound was prepared form the 4-(4-trifluoremethylphenoxy)aniline in a similar manner as described herein above for Example 3. MS (EI, m/z): 532 (M⁺+1, 100).

### Example 82

### N-Hydroxy (2S)-2-[(2S)-2-methoxymethylpyrrolidin-1-yl]-3-{N-[4-(4-trifluoromethylphenoxy)phenyl],N-methanesulfonylamino}propionamide

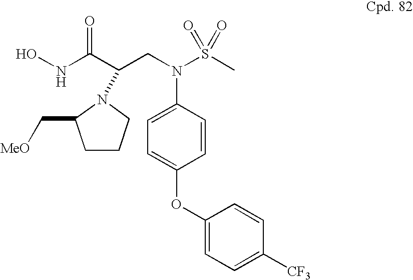

The title compound was prepared form the 4-(4-trifluoremethylphenoxy)aniline in a similar manner as described herein above for Example 3. MS (EI, m/z): 532 (M⁺+1, 100).

### Example 83

### N-Hydroxy (2S)-2-(4-methanesulfonylpiperazin-1-yl)-3-{N-[4-(4-trifluoromethylphenoxy)phenyl],N-methanesulfonylamino}propionamide

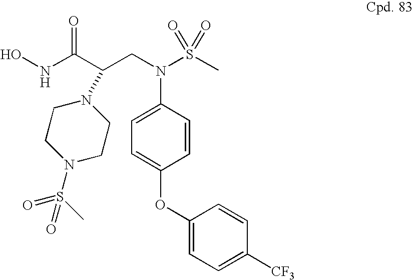

The title compound was prepared form the 4-(4-trifluoremethylphenoxy)aniline in a similar manner as described herein above for Example 3. MS (EI, m/z): 581 (M⁺+1, 100).

### Example 84

### N-Hydroxy (2S)-2-(4-acetylpiperazin-1-yl)-3-{N-[4-(4-trifluoromethylphenoxy)phenyl],N-methanesulfonylamino}propionamide

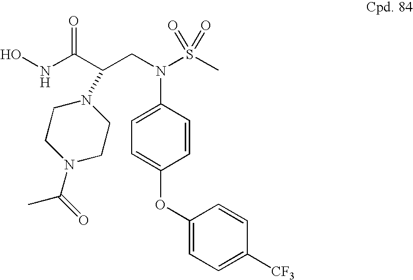

The title compound was prepared form the 4-(4-trifluoremethylphenoxy)aniline in a similar manner as described herein above for Example 3. MS (EI, m/z): 567 (M⁺+Na), 545 (M⁺+1, 100).

### Example 85

### N-Hydroxy (2S)-2-[4-(N-methylaminocarbonyl)piperidin-1-yl]-3-{N-[4-(4-trifluoromethylphenoxy)phenyl],N-methanesulfonylamino}propionamide

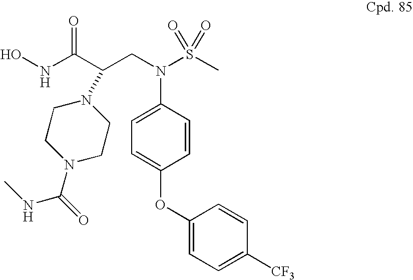

The title compound was prepared form the 4-(4-trifluoremethylphenoxy)aniline in a similar manner as described herein above for Example 3. MS (EI, m/z): 581 (M⁺+Na), 559 (M⁺+1, 100).

### Example 86

### N-Hydroxy (2S)-2-(cis-2,6-dimethylmorpholin-4-yl)-3-{N-[4-(4-trifluoromethylphenoxy)phenyl],N-methanesulfonylamino}propionamide

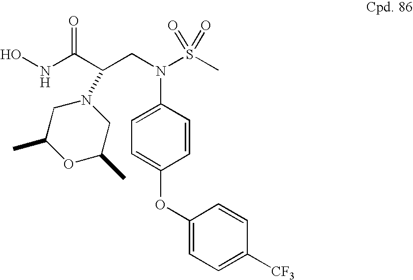

The title compound was prepared form the 4-(4-trifluoremethylphenoxy)aniline in a similar manner as described herein above for Example 3. MS (EI, m/z): 532 (M⁺+1, 100).

### Example 87

### N-Hydroxy (2S)-2-(3-oxopiperazin-1-yl)-3-{N-[4-(4-trifluoromethylphenoxy)phenyl],N-methanesulfonylamino}propionamide

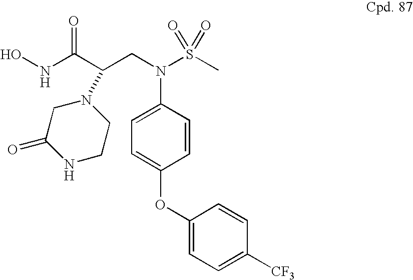

The title compound was prepared form the 4-(4-trifluoremethylphenoxy)aniline in a similar manner as described herein above for Example 3. MS (EI, m/z): 539 (M⁺+Na), 517 (M⁺+1, 100).

### Example 88

### N-Hydroxy (2S)-3-{N-[4-(4-chlorophenoxy)phenyl],N-methanesulfonylamino}-2-(4-hydroxypiperidin-1-yl)propionamide

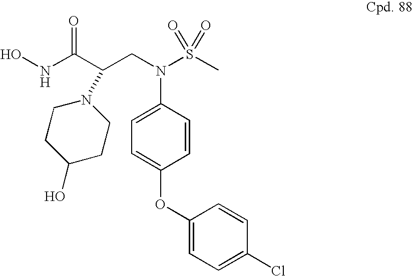

The title compound was prepared form the 4-(4-chlorophenoxy)aniline in a similar manner as described herein above for Example 3. MS (EI, m/z): 485 (M⁺+1), 483 (M⁺+1, 100).

### Example 89

### N-Hydroxy (2S)-2-(morpholin-4-yl)-3-{N-[4-(4-chlorophenoxy)phenyl],N-methanesulfonylamino}propionamide

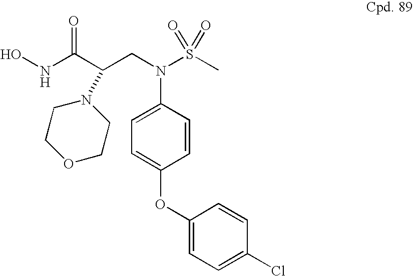

The title compound was prepared form the corresponding methyl ester in a similar manner as described herein above for Example 3. MS (EI, m/z): 471 (M⁺+1), 469 (M⁺+1, 100).

### Example 90

### N-Hydroxy (2S)-2-hydroxy-3-{N-[4-(4-fluorothiophenoxy)phenyl],N-methanesulfonylamino}propionamide

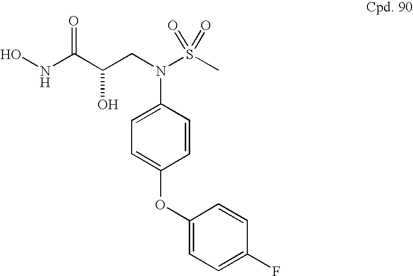

The title compound was prepared form the 4-(4-fluorothiophenoxy)aniline in a similar manner as described herein above for Example 1. MS (EI, m/z): 423 (M⁺+Na, 100), 401 (M⁺+1).

### Example 91

### N-Hydroxy (2S)-3-{N-[4-(4-chlorophenoxy)phenyl],N-methanesulfonylamino}-2-[4-(2-propyl)piperazin-1-yl]Propionamide

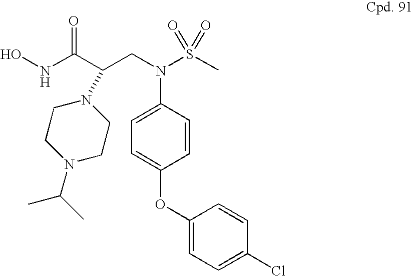

The title compound was prepared form the 4-(4-chlorophenoxy)aniline in a similar manner as described herein above for Example 3. MS (EI, m/z): 511 (M⁺+1, 100).

### Example 92

### N-Hydroxy (2S)-3-{N-[4-(4-chlorophenoxy)phenyl],N-methanesulfonylamino}-2-(4-methylpiperazin-1-yl)propionamide

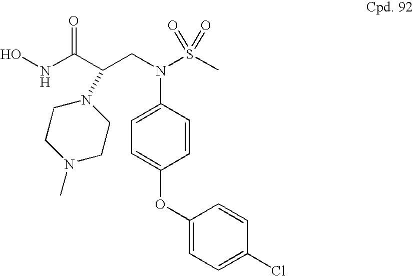

The title compound was prepared form the 4-(4-chlorophenoxy)aniline in a similar manner as described herein above for Example 3. MS (EI, m/z): 505 (M⁺+Na), 583 (M⁺+1, 100).

### Example 93

### N-Hydroxy (2S)-2-[(tetrahydropyran-4-yl)carbonylamino]-3-{N-[4-(4-trifluoromethylphenoxy)phenyl],N-methanesulfonylamino}propionamide

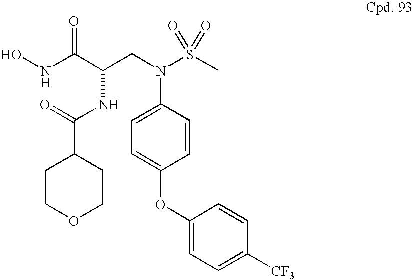

The title compound was prepared form the 4-(4-trifluoremethylphenoxy)aniline in a similar manner as described herein above for Example 4. ¹H NMR (300 MHz, d₆-DMSO) δ 10.72 (s, 1H), 8.90 (s, 1H), 7.89 (d, J=9.0 Hz, 1H), 7.77 (d, J=9.0 Hz, 2H), 7.40 (d, J=9.0 Hz, 2H), 7.21 (d, J=9.0 Hz, 2H), 7.15 (d, J=9.0 Hz, 2H), 4.30-4.20 (m, 1H), 3.90-3.75 (m, 4H), 3.31-3.22 (m, 2H), 3.00 (s, 3H), 2.45-2.35 (m, 1H), 1.60-1.45 (m, 4H); MS (EI, m/z): 568 (M⁺+Na), 546 (M⁺+1). Anal. Calcd for C₂₃H₂₆F₃N₃O₇S: C, 50.64; H, 4.80; N, 7.70. Found: C, 50.23; H, 4.68; N, 7.23.

### Example 94

### N-Hydroxy (2S)-3-{N-[4-(4-methylpentyn-1-yl)phenyl],N-methanesulfonylamino}-2-(morpholin-4-yl)propionamide

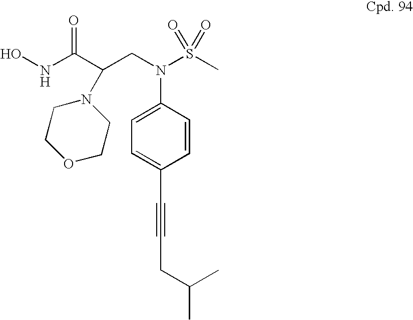

The title compound was prepared from Methyl (2S)-3-[N-(4-iodophenyl),N-methanesulfonylamino]-2-(morpholin-4-yl)propionate in a similar manner as described for Example 61, except using the 4-methyl-1-pentyne as the cross-coupling reagent in Step-1. ¹H NMR (300 MHz, d₆-DMSO) δ 10.75 (br s, 1H), 7.43 (d, J=9.0 Hz, 2H), 7.36 (d, J=9.0 Hz, 2H), 4.06 (dd, J=15.0, 6.0 Hz, 1H), 3.84 (dd, J=15.0, 6.0 Hz, 1H), 3.65-3.45 (m, 4H), 3.15-3.05 (m, 1H), 3.00 (s, 3H), 2.70-2.60 (m, 4H), 2.34 (d, J=6.0, 2H), 1.86 (quint, J=6.0 Hz, 1H), 1.01 (d, J=6.0 Hz, 6H); MS (EI, m/z): 424 (M⁺+1, 100).

### Example 95

### N-Hydroxy (2S)-3-{N-[4-(4-chlorophenoxy)phenyl],N-methanesulfonylamino}-2-(cis-2,6-dimethylmorpholin-4-yl)propionamide

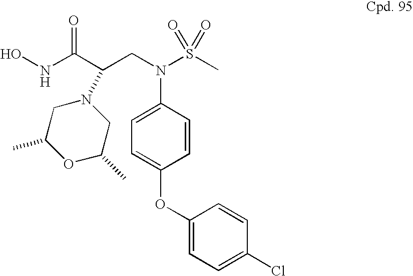

The title compound was prepared form the 4-(4-chlorophenoxy)aniline in a similar manner as described herein above for Example 3. MS (EI, m/z): 500 (M⁺+1), 498 (M⁺+1, 100).

### Example 96

### N-Hydroxy (2S)-2-(piperidin-1-yl)-3-{N-[4-(4-trifluoromethylphenoxy)phenyl],N-methanesulfonylamino}propionamide

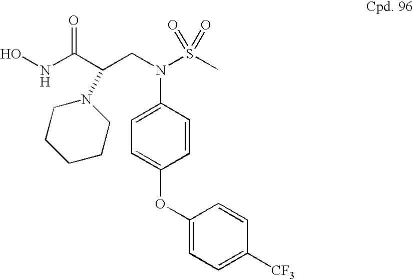

The title compound was prepared form the 4-(4-trifluoromethylphenoxy)aniline in a similar manner as described herein above for Example 3. MS (EI, m/z): 502 (M⁺+1, 100).

### Example 97

### N-Hydroxy (2S)-2-[N-methyl,N-(2-prolyl)amino]-3-{N-[4-(4-trifluoromethylphenoxy)phenyl],N-methanesulfonylamino}propionamide

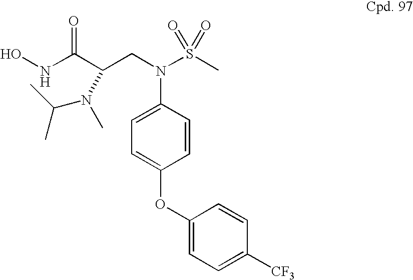

The title compound was prepared form the 4-(4-trifluoromethylphenoxy)aniline in a similar manner as described herein above for Example 3. MS (EI, m/z): 490 (M⁺+1, 100).

### Example 98

### N-Hydroxy,N-methyl (2S)-2-(morpholin-4-yl)-3-{N-[4-(4-trifluoromethylphenoxy)phenyl],N-methanesulfonylamino}propionamide

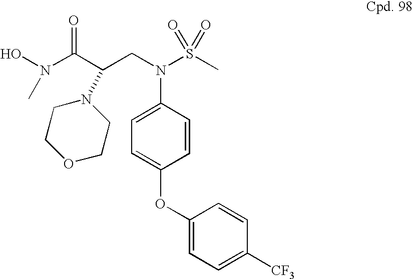

The title compound was prepared from the 4-(4-trifluoromethylphenoxy)aniline in a similar manner as described herein above for Example 5. MS (EI, m/z): 518 (M⁺+1, 100).

### Example 99

### N-Hydroxy (2S)-2-(2-propoxy)-3-{N-[4-(4-trifluoromethyl phenoxy)phenyl],N-methanesulfonylamino}propionamide

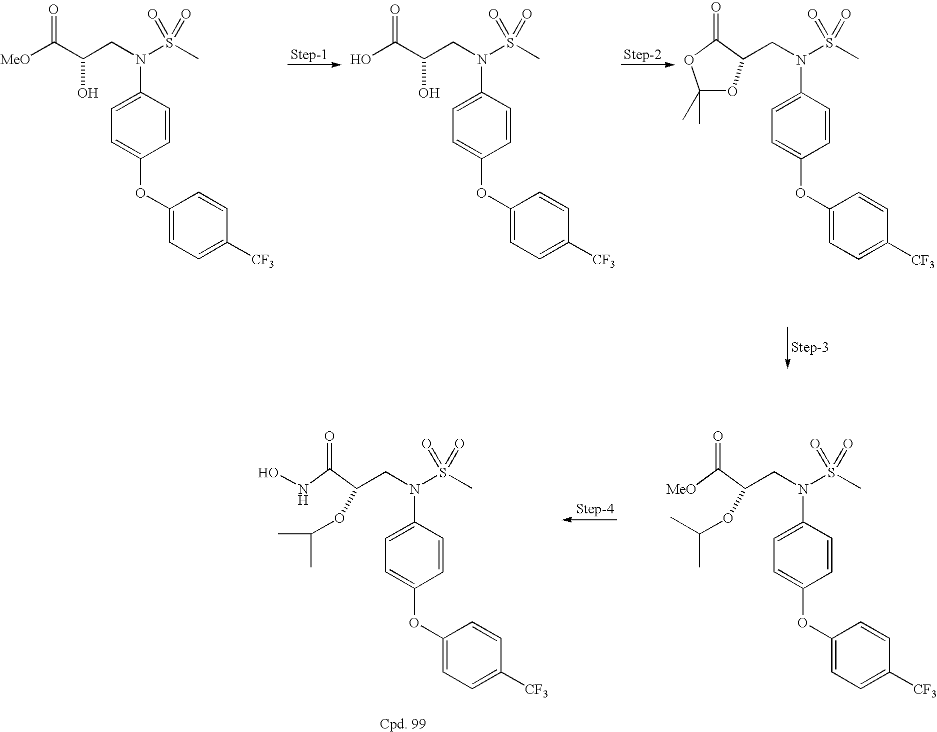

The compound, Methyl (2S)-2-hydroxy-3-{N-[4-(4-trifluoromethylphenoxy)phenyl],N-methanesulfonylamino}propionate, was prepared from 4-(4-trifluoromethylphenoxy)aniline in accordance with the sequence of general procedure I and II (GP-I and GP-II) as described herein above for reference examples.

Step-1:

To a solution of Methyl (2S)-2-hydroxy-3-{N-[4-(4-trifluoromethylphenoxy)phenyl],N-methanesulfonylamino}propionate (866 mg, 2.0 mmol) in THF (10 mL) was added LiOH_{(aq)} (1 N, 4 mL, 4 mmol) at room temperature. The mixture was stirred at rt for 1 h and was poured into EtOAc/H₂O (100 mL/100 mL). HCl_{(aq)} (2 N, 10 mL) was added and the organic layer was washed with H₂O (100 mL), brine (100 mL), dried (Na₂SO₄), and filtered. After removal of solvent, the product was dried to give 825 mg of (2S)-2-Hydroxy-3-{N-[4-(4-trifluoromethylphenoxy)phenyl],N-methanesulfonylamino}propionic acid (98%) as a white solid. ¹H NMR (300 MHz, d₆-DMSO) δ 12.68 (br s, 1H), 7.76 (d, J=9.0 Hz, 2H), 7.68 (d, J=9.0 Hz, 2H), 7.11 (d, J=9.0 Hz, 2H), 7.16 (d, J=9.0 Hz, 2H), 5.62 (br s, 1H), 3.95-3.85 (m, 2H), 3.75 (dd, J=15.0, 9.0 Hz, 1H), 3.05 (s, 3H); MS (EI, m/z): 418 (M⁺−1, 100).

Step-2:

To a suspension of (2S)-2-Hydroxy-3-{N-[4-(4-trifluoromethylphenoxy)phenyl],N-methanesulfonylamino}propionic acid (825 g, ca. 2.0 mmol) in 2,2-dimethoxypropane (6 mL) was added p-toluenesulfonic acid mnonhydrate (3.8 mg, 0.02 mmol) at room temperature. The mixture was stirred at rt for 6 h and was poured into Et₂O/H₂O (100 mL/100 mL). Saturated NaHCO_{3(aq)} (20 mL) was added. The organic layer was washed with brine (100 mL), dried (Na₂SO₄), and filtered. After removal of solvent, the crude product was purified by silica gel chromatography using EtOAc/hexane (1/4 to 2/3) as the eluent to give 650 mg of N-{[(5S)-2,2-dimethyl-4-oxo-1,3-dioxolan-5-yl]methyl},N-[4-(4-trifluoromethylphenoxy)phenyl]methanesulfonamide (71%) as a white solid. ¹H NMR (300 MHz, CDCl₃) δ 7.62 (d, J=9.0 Hz, 2H), 7.38 (d, J=9.0 Hz, 2H), 7.11 (d, J=9.0 Hz, 2H), 7.07 (d, J=9.0 Hz, 2H), 4.49 (dd, J=6.0, 3.0 Hz, 1H), 4.14-4.03 (m, 2H), 3.02 (s, 3H), 1.64 (s, 3H), 1.56 (s, 3H).

Step-3:

To a solution of N-{[(5S)-2,2-dimethyl-4-oxo-1,3-dioxolan-5-yl]methyl},N-[4-(4-trifluoromethylphenoxy)phenyl]methanesulfonamide (459 mg, 1.0 mmol) in CH₂Cl₂ (10 mL) at −78° C. under N₂ was added triethylsilane (0.48 mL, 3.0 mmol) and then TiCl₄ (1.0 M in CH₂Cl₂, 2.0 mL, 2.0 mmol). The mixture was stirred at −78° C. for 30 min and methanol (10 mL) was added. The mixture was allowed to warm to rt and was stirred for 16 h. The mixture was poured into Et₂O/H₂O (100 mL/100 mL). The organic layer was washed with H₂O (100 mL), brine (100 mL), dried (Na₂SO₄), and filtered. After removal of solvent, the crude product was purified by silica gel chromatography using EtOAc/hexane (1/4 to 3/7) as the eluent to give 409 mg of Methyl (2S)-2-(2-propoxy)-3-{N-[4-(4-trifluoromethylphenoxy)phenyl],N-methanesulfonylamino}propionate (86%) as a white solid. ¹H NMR (300 MHz, CDCl₃) δ 7.60 (d, J=9.0 Hz, 2H), 7.35 (dd, J=9.0, 3.0 Hz, 2H), 7.08 (d, J=9.0 Hz, 2H), 7.04 (dd, J=9.0, 3.0 Hz, 2H), 4.15 (dd, J=6.0, 3.0 Hz, 1H), 4.01 (dd, J=15.0, 3.0 Hz, 1H), 3.92 (dd, J=15.0, 6.0 Hz, 1H), 3.73-3.65 (m, 1H), 3.68 (s, 3H), 3.00 (s, 3H), 1.17 (d, J=6.0 Hz, 3H), 1.13 (d, J=6.0 Hz, 3H).

Step-4:

To a mixture of Methyl (2S)-2-(2-propoxy)-3-{N-[4-(4-trifluoromethylphenoxy)phenyl],N-methanesulfonylamino}propionate (238 mg, 0.5 mmol) and hydroxylamine hydrochloride (140 mg, 2.0 mmol) in dried MeOH (1 mL) under N₂ at room temperature was added sodium methoxide (0.5 M in MeOH, 6.0 mL, 3.0 mmol). The mixture was stirred at room temperature for 2 h and was poured into EtOAc/H₂O (100 mL/100 mL) and HCl_{(aq)} (2 N, 5 mL) was added. The aqueous layer was extracted with EtOAc (50 mL). The combined organic layers was dried (Na₂SO₄) and filtered. After removal of solvent, the product was slightly purified by silica gel chromatography using EtOAc/hexane (2/3 to 4/1 to 1/0) as the eluent to give 169 mg of N-Hydroxy (2S)-2-(2-propoxy)-3-{N-[4-(4-trifluoromethylphenoxy)phenyl],N-methanesulfonylamino}propionamide (71%) as a white solid. ¹H NMR (300 MHz, CDCl₃) δ 8.99 (br s, 1H), 7.95 (br s, 1H), 7.61 (d, J=9.0 Hz, 2H), 7.38 (dd, J=9.0, 3.0 Hz, 2H), 7.11-7.03 (m, 4H), 4.13-4.06 (m, 2H), 3.96 (dd, J=15.0, 6.0 Hz, 1H), 3.76 (quint, J=6.0 Hz, 1H), 2.94 (s, 3H), 1.7 (d, J=6.0 Hz, 3H), 1.5 (d, J=6.0 Hz, 3H); MS (EI, m/z): 499 (M⁺+Na), 477 (M⁺+1, 100).

### Example 100

### N-Hydroxy (2S)-2-(cis-2,6-dimethylmorpholin-4-yl)-3-{N-[4-(4-methylphenoxy)phenyl],N-methanesulfonylamino}propionamide

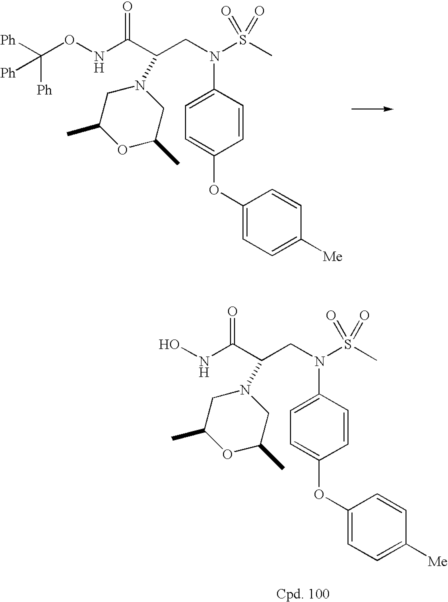

The compound, N-Triphenylmethoxy (2S)-2-(cis-2,6-dimethylmorpholin-4-yl)-3-{N-[4-(4-methylphenoxy)phenyl],N-methanesulfonylamino}propionamide, was prepared from 4-(4-methylphenoxy)aniline in accordance with the sequence of general procedure I, II, III, IV, V, and VI (GP-I, GP-II, GP-VIII, GP-IV, GP-V, and GP-VI) as described herein above for reference examples.

To a suspension of N-Triphenylmethoxy (2S)-2-(cis-2,6-dimethylmorpholin-4-yl)-3-{N-[4-(4-methylphenoxy)phenyl],N-methanesulfonylamino}propionamide (8.98 g, 13 mmol) in Et₂O (10 mL) at room temperature was added trifluoroacetic acid (10 mL) slowly. After 10 min stirring, pure H₂O (10 mL) was added and was stirred for another 1 h. The mixture was poured into vigorously stirring H₂O/Et₂O/hexane (100 mL/50 mL/50 mL). The white suspension was filtered and was washed with Et₂O/hexane (1/1, 100 mL). The white solid was recrystallized from Et₂O/hexane to give 5.81 g of product. The aqueous layer was lyophilized and the product was recrystallized from Et₂O/hexane to give 0.31 g of product. Overall, 6.12 g of N-Hydroxy (2S)-2-(cis-2,6-dimethylmorpholin-4-yl)-3-{N-[4-(4-methylphenoxy)phenyl],N-methanesulfonylamino}propionamide (84%) was obtained as a white trifluoroacetic acid salt. MS (EI, m/z): 478 (M⁺+1, 100)

### Example 101

### N-Hydroxy,N-[2-(morpholin-4-yl)ethyl](2S)-2-hydroxy-3-{N-[4-(4-chlorophenoxy)phenyl],N-methanesulfonylamino}propionamide

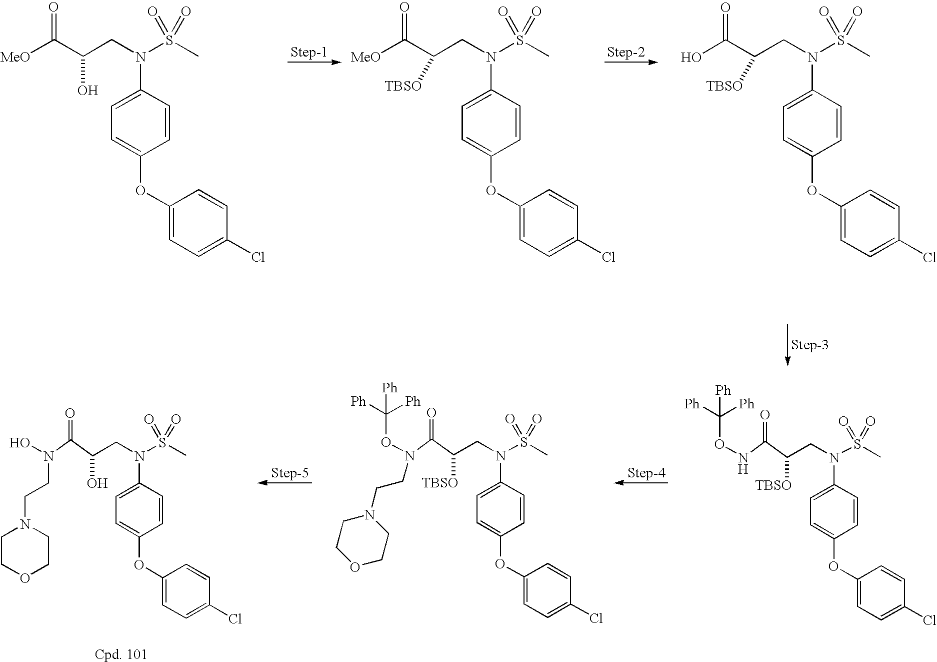

The compound, Methyl (2S)-2-hydroxy-3-{N-[4-(4-chlorophenoxy)phenyl],N-methanesulfonylamino}propionate, was prepared from 4-(4-chlorophenoxy)aniline in accordance with the sequence of general procedure I and II (GP-I and GP-II) as described herein above for reference examples.

Step-1:

To a solution of Methyl (2S)-2-hydroxy-3-{N-[4-(4-chlorophenoxy)phenyl],N-methanesulfonylamino}propionate (2.0 g, 5.0 mmol) in DMF (2 mL) was added pyridine (2 mL) and then tert-butyldimethylsilyl chloride (900 mg, 6.0 mmol) at room temperature. The mixture was stirred at rt for 2 days. The mixture was poured into Et₂O/H₂O (150 mL/150 mL) and HCl_{(aq)} (2 N, 25 mL) was added. The organic layer was washed with H₂O (150 mL), brine (150 mL), dried (Na₂SO₄), and filtered. After removal of solvent, the product was dried in vacuo to give 2.15 g of Methyl (2S)-2-(tert-butyl-dimethylsilyloxy)-3-{N-[4-(4-chlorophenoxy)phenyl],N-methanesulfonylamino}propionate (84%), which was used without further purification. ¹H NMR (300 MHz, CDCl₃) δ 7.30-7.27 (m, 4H), 6.95-6.91 (m, 4H), 4.33 (d, J=6.0 Hz, 1H), 4.98 (dd, J=12.0, 6.0 Hz, 1H), 3.89 (dd, J=12.0, 6.0 Hz, 1H), 3.61 (s, 3H), 2.90 (s, 3H), 0.83 (s, 9H), 0.01 (s, 3H), 0.00 (s, 3H).

Step-2:

To a solution of Methyl (2S)-2-(tert-butyl-dimethylsilyloxy)-3-{N-[4-(4-chlorophenoxy)phenyl],N-methanesulfonylamino}propionate (2.1 g, 4.1 mmol) in THF (4 mL) was added LiOH_{(aq)} (1 N, 8.2 mL, 8.2 mmol) at room temperature. The mixture was stirred at rt for 3 h and was poured into EtOAc/H₂O (150 mL/150 mL). HCl_{(aq)} (2 N, 10 mL) was added. The organic layer was washed with H₂O (100 mL), dried (Na₂SO₄), and filtered. After removal of solvent, the product was resolidified from Et2O/hexane and removal of Et₂O slowly to give 1.7 g of (2S)-2-(tert-butyl-dimethylsilyloxy)-3-{N-[4-(4-chlorophenoxy)phenyl],N-methanesulfonylamino}propionic acid (83%) as a white solid. ¹H NMR (300 MHz, CDCl₃) δ 7.25-7.22 (m, 4H), 6.89-6.86 (m, 4H), 4.86 (t, J=6.0 Hz, 1H), 3.96 (dd, J=12.0, 3.0 Hz, 1H), 3.89 (dd, J=12.0, 6.0 Hz, 1H), 2.83 (s, 3H), 0.79 (s, 9H), 0.02 (s, 3H), 0.00 (s, 3H); MS (EI, m/z): 500 (M⁺−1), 499 (M⁺−1, 100).

Step-3:

To a mixture of (2S)-2-(tert-butyl-dimethylsilyloxy)-3-{N-[4-(4-chlorophenoxy)phenyl],N-methanesulfonylamino}propionic acid (998 mg, 2.0 mmol), EDC (573 mg, 3.0 mmol), HOBt (405 mg, 3.0 mmol), and 4-N,N-dimethylaminopyridine (366 mg, 3.0 mmol) in CHCl₃ (10 mL) was added Et₃N (0.42 mL, 3.0 mmol) under N₂ at room temperature. After 1 h stirring, O-tritylhydroxylamine (825 mg, 3.0 mmol) was added and was stirred for 5 h at rt. The mixture was then poured into Et₂O/H₂O (100 mL/100 mL). The organic layer was washed with H₂O (100 mL), brine (100 mL), dried (Na₂SO₄), and filtered. After removal of solvent, the crude mixture was purified by silica gel chromatography using EtOAc/hexane (3/7 to 3/2) as eluent to give 785 mg of N-Triphenyl methoxy (2S)-2-(tert-butyl-dimethylsilyloxy)-3-{N-[4-(4-chlorophenoxy)phenyl],N-methanesulfonylamino}propionamide (52%) as a white solid. ¹H NMR (300 MHz, CDCl₃) δ 8.34 (s, 1H), 7.43-7.23 (m, 19H), 6.96-6.91 (m, 4H), 4.06 (t, J=6.0 Hz, 1H), 3.78 (d, J=6.0 Hz, 2H), 2.84 (s, 3H), 0.67 (s, 9H), 0.00 (s, 3H), −0.02 (s, 3H); MS (EI, m/z): 757 (M⁺−1), 755 (M⁺−1, 100).

Step-4:

The mixture of 4-(2-chloroethyl)morpholine (74 mg, 0.4 mmol) and sodium iodide (60 mg, 0.4 mmol) in DMF (2 mL) was stirred at room temperature for 15 min. Then, N-Triphenylmethoxy (2S)-2-(tert-butyl-dimethylsilyloxy)-3-{N-[4-(4-chlorophenoxy)phenyl],N-methanesulfonylamino}propionamide (152 mg, 0.2 mmol) and Cs₂CO₃ (260 mg, 0.8 mmol) were added sequentially. The mixture was stirred at rt for 1 h and was then heated at 60° C. for 24 h. The mixture was poured into Et₂O/H₂O (50 mL/50 mL). The organic layer was washed with brine (50 mL), dried (Na₂SO₄), and filtered. After removal of solvent, the product was purified by silica gel chromatography using EtOAc/hexane (3/7 to 1/1) as the eluent to give 110 mg of N-(Morpholin-4-yl)ethyl,N-triphenylmethoxy (2S)-2-(tert-butyl-dimethylsilyloxy)-3-{N-[4-(4-chlorophenoxy)phenyl],N-methanesulfonylamino}propionamide (63%) as a sticky oil. ¹H NMR (300 MHz, CDCl₃) δ 7.37-7.23 (m, 17H), 6.95-6.78 (m, 6H), 4.70-4.60 (m, 1H), 4.55-4.45 (m, 1H), 3.97-3.91 (m, 2H), 3.69-3.66 (m, 4H), 3.48 (dd, J=12.0, 3.0 Hz, 1H), 2.72 (s, 3H), 2.63 (t, J=6.0 Hz, 1H), 2.51-2.48 (m, 4H), 0.76 (s, 9H), −0.18 (s, 6H); MS (EI, m/z): 872 (M⁺+1), 869 (M⁺+1, 100).

Step-5:

To a solution of N-(Morpholin-4-yl)ethyl,N-triphenylmethoxy (2S)-2-(tert-butyl-dimethylsilyloxy)-3-{N-[4-(4-chlorophenoxy)phenyl],N-methanesulfonylamino}propionamide (100 mg, 0.12 mmol) in THF (1 mL) at room temperature was added tetrabutylammonium fluoride (1.0 M in THF, 0.55 mL, 0.55 mmol). The mixture was stirred at rt for 15 min and was then poured into Et₂O/H₂O (50 mL/50 mL). The organic layer was washed with brine (50 mL), dried (Na₂SO₄), and filtered. After removal of solvent, the product was purified by silica gel chromatography using EtOAc/hexane (2/3 to 4/1) as the eluent to give 65 mg of product (75%) as a sticky oil. ¹H NMR (300 MHz, CDCl₃) δ 7.36-7.22 (m, 17H), 7.11 (d, J=9.0 Hz, 2H), 6.95 (d, J=9.0 Hz, 2H), 6.84 (d, J=9.0 Hz, 2H), 6.73 (br s, 1H), 4.65-4.58 (m, 1H), 4.44-4.37 (m, 1H), 3.94 (t, J=6.0 Hz, 1H), 3.77 (dd, J=12.0, 6.0 Hz, 1H), 3.66 (dd, J=12.0, 6.0 Hz, 1H), 3.57-3.54 (m, 4H), 2.93 (s, 3H), 2.53-2.23 (m, 6H); MS (EI, m/z): 758 (M⁺+1), 756 (M⁺+1, 100). This product (60 mg, 0.079 mmol) was dissolved in Et₂O (1 mL) and then trifluoroacetic acid (1 mL) was added. After 5 min stirring at room temperature, pure H₂O (1 mL) was added and the mixture was stirred at rt for 1 h. The mixture was poured into Et₂O/H₂O (25 mL/50 mL) and hexane (25 mL) was added. The organic layer was extracted with pure water (50 mL). The aqueous layers were combined and lyophilized to give 38 mg of N-Hydroxy,N-(morpholin-4-yl)ethyl (2S)-2-hydroxy-3-{N-[4-(4-chlorophenoxy)phenyl], N-methanesulfonylamino}propionamide (77%) as a trifluoroacetic acid salt. ¹H NMR (300 MHz, CDCl₃) δ 7.35 (d, J=9.0 Hz, 2H), 7.31 (d, J=9.0 Hz, 2H), 7.01-6.96 (m, 4H), 4.52 (br m, 2H), 4.30 (br m, 1H), 4.15-3.90 (m, 10H), 3.45-3.35 (m, 2H), 2.96 (s, 3H).

### Example 102

### N-(3-Allyl),N-hydroxy (2S)-2-(cis-2,6-dimethylmorpholin-4-yl)-3-{N-[4-(4-methylphenoxy)phenyl],N-methanesulfonylamino}propionamide

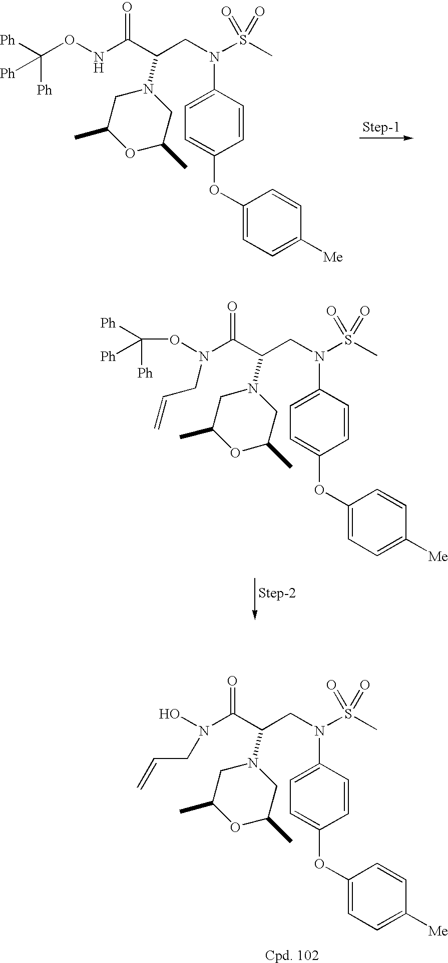 Step-1:

In a three-neck flask was placed N-Triphenylmethoxy (2S)-2-(cis-2,6-dimethylmorpholin-4-yl)-3-{N-[4-(4-methylphenoxy)phenyl],N-methanesulfonylamino}propionamide (216 mg, 0.3 mmol). The air was removed under vacuum and was refilled with N₂. Then, MeCN (2 mL), allyl methyl carbonate (0.068 mL, 0.6 mmol), and Pd(PPh₃)₄ (7 mg, 0.006 mmol) were added sequentially. The mixture was stirred at room temperature for 30 min and was poured into EtOAc/H₂O (50 mL/50 mL). The organic layer was dried (Na₂SO₄) and filtered. After removal of solvent, the product was purified by silica gel chromatography using EtOAc/hexane (1/4 to 2/3) as the eluent to give 199 mg of N-(3-Allyl),N-triphenylmethoxy (2S)-2-(cis-2,6-dimethylmorpholin-4-yl)-3-{N-[4-(4-methylphenoxy)phenyl],N-methanesulfonylamino}propionamide (87%).

Step-2:

To a suspension of N-(3-Allyl),N-triphenylmethoxy (2S)-2-(cis-2,6-dimethylmorpholin-4-yl)-3-{N-[4-(4-methylphenoxy)phenyl],N-methanesulfonylamino}propionamide (152 mg, 0.2 mmol) in CH₂Cl₂/MeOH (0.8 mL/0.2 mL) at room temperature was added BF₃.OEt₂ (0.075 mL, 0.6 mmol). After 10 min stirring, the mixture was poured into EtOAc/H₂O (50 mL/50 mL). The organic layer was washed with brine (50 mL), dried (Na₂SO₄), and filtered. After removal of solvent, Et₂O (5 mL) and then hexane (25 mL) were added sequentially. The solid was filtered and was washed with Et₂O/hexane (3/17, 10 mL) to give 66.5 mg of N-(3-Allyl),N-hydroxy (2S)-2-(cis-2,6-dimethylmorpholin-4-yl)-3-{N-[4-(4-methylphenoxy)phenyl],N-methanesulfonylamino}propionamide (64%) as a white solid. MS (EI, m/z): 518 (M⁺+1, 100)

MMP Dose Response Assay for Determining IC₅₀

The following method was used for determining IC₅₀ values of different inhibitor compounds against multiple MMP enzymes. Frozen stocks of recombinantly produced MMPs were prepared in assay buffer (50 mM HEPES pH 7.4, 10 mM CaCl₂, 0.05% Brij-35). The following domains were used to produce catalytically active version of the indicated MMP enzymes, human MMP-1 (amino acids 100-262), human MMP-2 (amino acids 1-660), human MMP-3 (amino acids 100-265), human MMP-9 (amino acids 107-446), human MMP-13 (amino acids 103-268), and rat MMP-9 (amino acids 108-446). Enzyme concentrations in the assay were determined empirically and set such that proteolytic activity was maintained in a linear range over the time-course of the experiment. Diluted enzymes were mixed with various test compounds that had been pre-diluted into assay buffer to establish concentration curves ranging from 10⁻⁵ M to 10⁻¹² M. Compounds were diluted into assay buffer that contained an appropriate concentration of DMSO to maintain this at a constant in all wells. Each concentration for each test compound was tested in triplicate. MMP enzymes were equilibrated with compounds for 1 hour at room temperature, then a fluorescent-quench substrate (Acetyl-Cys(Eu)-Pro-Leu-Gly-Leu-Lys-(QSY7)-Ala-Arg-amide) was added to a final concentration of 100 nM. Following a 15 minute room temperature incubation, the assay plate was read on an EnVision plate reader using the following parameters: excitation wavelength 340 nM, emission wavelength 615 nM, number of flashes 100, delay before reading 300 milliseconds. Data was analyzed by first calculating average background value from wells that contained only substrate, and this value was subtracted from the entire plate. The 100%, or maximum value, was determined by averaging control wells that contained enzyme, substrate and DMSO. For each well, the percent of control was calculated by dividing the well value by the average DMSO value obtained above. IC₅₀ values were calculated by fitting percent of control values for each compound to a curve established by nonlinear regression with parameters of a sigmoidal dose-response curve having a variable slope with curves constrained from 0% to 100% of control.

### BIOLOGICAL EXAMPLES

Compounds listed in Table II below were tested in the above assay(s):

TABLE II

| Cpd. # | MMP-1 (nM) | MMP-2 (nM) | MMP-9 (nM) |
|---|---|---|---|
| 1 | >10000 | 39.5 | 5.04 |
| | | 30.3 | 4.35 |
| 2 | >10000 | 43.6 | 3.73 |
| | | 28.8 | 4.15 |
| 3 | | 49.8 | 18.4 |
| | | 17.1 | 7.46 |
| | | 13.5 | 4.06 |
| | | 9.33 | 3.53 |
| | | 7.57 | 2.37 |
| | | 8.99 | 2.76 |
| | | 2.02 | 7.64 |
| | | 6.17 | 2.89 |
| | | 8.19 | 2.08 |
| | | 11.5 | 2.79 |
| 4 | >10000 | 53.0 | 4.89 |
| | | 86.7 | 6.00 |
| 5 | | 261.2 | 87.9 |
| 6 | >10000 | 19.1 | 7.40 |
| | 5571.9 | 22.6 | 7.76 |
| | | 29.7 | 7.44 |
| | | 16.6 | 4.82 |
| | | 18.9 | 2.17 |
| | | 14.5 | 3.57 |
| 7 | >10000 | 81.7 | 20.6 |
| | | 52.0 | 3.13 |
| 8 | >10000 | 88.5 | 10.5 |
| | >10000 | 67.8 | 11.1 |
| | | 98.2 | 10.1 |
| | | 65.0 | 3.37 |
| | | 51.9 | 4.69 |
| | | 71.5 | 7.57 |
| 9 | | 83.6% | 78.7% |
| 10 | | 87.0% | 79.0% |
| 11 | >10000 | 472.0 | 105.7 |
| 12 | >10000 | 306.2 | 49.7 |
| | | 188.8 | 29.2 |
| 13 | 9682.8 | 24.1 | 5.57 |
| | 5432.5 | 44.9 | 7.16 |
| 14 | >10000 | 582.1 | 92.9 |
| 15 | >10000 | 509.3 | 123.9 |
| 16 | >10000 | 1233.1 | 286.4 |
| 17 | >10000 | 101.4 | 12.7 |
| 18 | >10000 | 3.63 | 0.61 |
| | 9564.0 | 2.51 | 0.75 |
| | 4168.7 | 3.25 | 0.30 |
| | 8394.6 | 5.11 | 0.41 |
| | 6950.2 | 5.46 | 0.61 |
| | | 4.21 | 0.45 |
| 19 | >10000 | 1056.8 | 243.2 |
| 20 | >10000 | 115.4 | 21.4 |
| | | 169.0 | 33.6 |
| 21 | >10000 | 2382.3 | 343.6 |
| 22 | >10000 | 210.4 | 10.8 |
| | | 342.0 | 41.6 |
| 23 | >10000 | 56.2 | 2.60 |
| | | 90.4 | 9.22 |
| 24 | >10000 | 187.1 | 37.8 |
| | >10000 | 167.9 | 24.9 |
| 25 | >10000 | 201.8 | 7.16 |
| 26 | >10000 | 54.8 | 93.3 |
| 27 | >10000 | 3.21 | 1.52 |
| | >10000 | 4.98 | 2.56 |
| | | 4.19 | 0.93 |
| | | 10.7 | 4.05 |
| | | 8.28 | 2.49 |
| 28 | >10000 | 1318.2 | 295.1 |
| 29 | >10000 | 669.9 | 120.5 |
| 30 | >10000 | 18.6 | 0.46 |
| | >10000 | 15.4 | 0.70 |
| | >10000 | 15.3 | 1.09 |
| | 6223.0 | 5.27 | 0.05 |
| | 8729.7 | 9.50 | 0.28 |
| | 7277.8 | 9.90 | 0.30 |
| | 7464.5 | 7.96 | 0.08 |
| | | 9.50 | 0.28 |
| 31 | >10000 | 344.4 | 54.2 |
| 32 | >10000 | 400.8 | 68.7 |
| 33 | >10000 | 9.53 | 1.77 |
| 34 | >10000 | 83.4 | 22.9 |
| 35 | >10000 | 16.8 | 2.84 |
| 36 | 9216.9 | 11.4 | 1.27 |
| 37 | >10000 | 9.98 | 1.69 |
| 38 | >10000 | 64.4 | 1.32 |
| 39 | >10000 | 34.7 | 0.46 |
| | >10000 | 50.6 | 1.18 |
| | >10000 | 73.1 | 1.68 |
| | >10000 | 53.0 | 0.25 |
| 40 | 6412.1 | 30.8 | 1.00 |
| | 8830.8 | 14.7 | 0.64 |
| 41 | >10000 | 126.8 | 2.56 |
| 42 | >10000 | 430.5 | 19.9 |
| 43 | >10000 | 335.7 | 22.0 |
| 44 | >10000 | 332.7 | 19.7 |
| 45 | 5997.9 | 682.3 | 58.3 |
| 46 | >10000 | 24.1 | 1.54 |
| 47 | 6792.0 | 11.7 | 0.54 |
| 48 | | 18.5 | 0.69 |
| 49 | | 274.2 | 62.8 |
| | | 320.6 | 44.5 |
| 50 | | 877.0 | 73.6 |
| 51 | | 26.8 | 0.62 |
| 52 | | 1076.5 | 164.8 |
| 53 | | 2535.1 | 472.1 |
| 54 | | 175.0 | 17.2 |
| | | 102.3 | 10.7 |
| 55 | | 1559.6 | 410.2 |
| 56 | | 396.3 | 143.9 |
| 57 | | 406.4 | 227.0 |
| 58 | | 9.16 | 2.37 |
| 59 | | 465.6 | 31.9 |
| 60 | 5984.1 | 1442.1 | 86.5 |
| | >10000 | 875.0 | 93.8 |
| 61 | >10000 | 31.6 | 91.0 |
| 62 | >10000 | 56.2 | 47.3 |
| 63 | >10000 | 28.2 | 5.21 |
| | | 65.9 | 83.2 |
| 64 | >10000 | 103.3 | 774.5 |
| 65 | >10000 | 0.37 | 0.65 |
| 66 | >10000 | 41.2 | 2.88 |
| 67 | >10000 | 4.76 | 6.52 |
| | >10000 | 4.37 | 8.51 |
| 68 | >10000 | 261.8 | 28.8 |
| 69 | >10000 | 281.2 | 67.5 |
| 70 | >10000 | 4.65 | 1.90 |
| | >10000 | 4.99 | 1.52 |
| | | 15.8 | 6.29 |
| 71 | 8550.6 | 1.57 | 0.66 |
| | >10000 | 4.93 | 1.45 |
| | | 10.71 | 3.56 |
| 72 | >10000 | 3.76 | 0.72 |
| | >10000 | 1.02 | 0.17 |
| 73 | >10000 | 1.10 | 0.02 |
| | >10000 | 5.65 | 0.23 |
| | >10000 | 28.8 | 6.73 |
| | | 5.25 | 0.23 |
| 74 | >10000 | 17.7 | 3.86 |
| 75 | >10000 | 15.2 | 2.18 |
| | >10000 | 21.5 | 3.16 |
| 76 | >10000 | 47.2 | 17.0 |
| 77 | >10000 | 118.9 | 256.4 |
| 78 | >10000 | 317.7 | 115.6 |
| 79 | >10000 | 38.9 | 37.1 |
| 80 | >10000 | 24.3 | 2.67 |
| 81 | >10000 | 14.2 | 3.16 |
| 82 | >10000 | 11.3 | 7.32 |
| 83 | >10000 | 6.21 | 1.94 |
| | | 13.5 | 4.50 |
| 84 | >10000 | 8.55 | 4.81 |
| 85 | >10000 | 5.79 | 3.48 |
| | | 5.16 | 1.51 |
| 86 | 8035.3 | 2.69 | 0.48 |
| | | 4.57 | 0.86 |
| | | 9.44 | 2.08 |
| | | 8.32 | 2.71 |
| | | 3.04 | 0.35 |
| 87 | >10000 | 19.8 | 7.94 |
| 88 | | 3.13 | 0.90 |
| 89 | | 5.52 | 2.00 |
| 90 | | 311.9 | 64.4 |
| | | 253.5 | 39.2 |
| 91 | | 8.77 | 3.11 |
| 92 | | 6.50 | 2.39 |
| 93 | | 835.6 | 54.1 |
| 94 | | 105.7 | 112.5 |
| 95 | | 2.54 | 0.57 |
| | | 3.69 | 0.64 |
| | | 1.28 | 0.17 |
| 96 | | 7.55 | 2.34 |
| 97 | | 17.4 | 5.48 |
| | | 29.9 | 12.2 |
| 98 | | 446.6 | 159.5 |
| 99 | | 50.3 | 8.18 |
| 100 | | 2.83 | 0.68 |
| 101 | | 1396.4 | 206.1 |
| 102 | | 23.0 | 6.53 |
| | | 24.8 | 6.01 |

% indicate the % inhibition at 10 μM

Additionally, compounds 27, 3, 65, 66, and 97 were tested in vitro for MMP-3, MMP-9 (rat), and MMP-13 antagonism. The results are shown in Table III

TABLE III

| | | MMP-3 | MMP-9 | MMP-13 |
|---|---|---|---|---|
| Cpd. # | JNJ-# | (nM) | (rat) (nM) | (nM) |
| 27 | | 136.5 | 25.8 | 3.53 |
| 3 | | 63.7 | | 30.5 |
| | | 104.5 | | 56.1 |
| 65 | | 492.0 | | 411.5 |
| 66 | | 822.2 | | 847.2 |
| 97 | | 217.6 | 174.8 | 12.4 |

The compounds of the present invention can be administered to a subject systemically, for example, intravenously, orally, subcutaneously, intramuscular, intradermal, or parenterally. The compounds of the present invention can also be administered to a subject locally. Non-limiting examples of local delivery systems include the use of intraluminal medical devices that include intravascular drug delivery catheters, wires, pharmacological stents and endoluminal paving. The compounds of the present invention can further be administered to a subject in combination with a targeting agent to achieve high local concentration of the compound at the target site. In addition, the compounds of the present invention may be formulated for fast-release or slow-release with the objective of maintaining the drugs or agents in contact with target tissues for a period ranging from hours to weeks.

The present invention also provides a pharmaceutical composition comprising a compound of Formula I in association with a pharmaceutically acceptable carrier. The pharmaceutical composition may contain between about 0.1 mg and 1000 mg, preferably about 100 to 500 mg, of the compound, and may be constituted into any form suitable for the mode of administration selected.

The phrase “pharmaceutically acceptable” refers to molecular entities and compositions that do not produce an adverse, allergic or other untoward reaction when administered to an animal, or a human, as appropriate. Veterinary uses are equally included within the invention and “pharmaceutically acceptable” formulations include formulations for both clinical and/or veterinary use.

Carriers include necessary and inert pharmaceutical excipients, including, but not limited to, binders, suspending agents, lubricants, flavorants, sweeteners, preservatives, dyes, and coatings. Compositions suitable for oral administration include solid forms, such as pills, tablets, caplets, capsules (each including immediate release, timed release and sustained release formulations), granules, and powders, and liquid forms, such as solutions, syrups, elixirs, emulsions, and suspensions. Forms useful for parenteral administration include sterile solutions, emulsions and suspensions.

The pharmaceutical composition of the present invention also includes a pharmaceutical composition for slow release of a compound of the present invention. The composition includes a slow release carrier (typically, a polymeric carrier) and a compound of the present invention.

Slow release biodegradable carriers are well known in the art. These are materials that may form particles that capture therein an active compound(s) and slowly degrade/dissolve under a suitable environment (e.g., aqueous, acidic, basic, etc) and thereby degrade/dissolve in body fluids and release the active compound(s) therein. The particles are preferably nanoparticles (i.e., in the range of about 1 to 500 nm in diameter, preferably about 50-200 nm in diameter, and most preferably about 100 nm in diameter).

The present invention also provides methods to prepare the pharmaceutical compositions of this invention. The compound of Formula I, as the active ingredient, is intimately admixed with a pharmaceutical carrier according to conventional pharmaceutical compounding techniques, which carrier may take a wide variety of forms depending on the form of preparation desired for administration, e.g., oral or parenteral such as intramuscular. In preparing the compositions in oral dosage form, any of the usual pharmaceutical media may be employed. Thus, for liquid oral preparations, such as for example, suspensions, elixirs and solutions, suitable carriers and additives include water, glycols, oils, alcohols, flavoring agents, preservatives, coloring agents and the like; for solid oral preparations such as, for example, powders, capsules, caplets, gelcaps and tablets, suitable carriers and additives include starches, sugars, diluents, granulating agents, lubricants, binders, disintegrating agents and the like. Because of their ease in administration, tablets and capsules represent the most advantageous oral dosage unit form, in which case solid pharmaceutical carriers are obviously employed. If desired, tablets may be sugar coated or enteric coated by standard techniques. For parenterals, the carrier will usually comprise sterile water, though other ingredients, for example, for purposes such as aiding solubility or for preservation, may be included. Injectable suspensions may also be prepared, in which case appropriate liquid carriers, suspending agents and the like may be employed. In preparation for slow release, a slow release carrier, typically a polymeric carrier, and a compound of the present invention are first dissolved or dispersed in an organic solvent. The obtained organic solution is then added into an aqueous solution to obtain an oil-in-water-type emulsion. Preferably, the aqueous solution includes surface-active agent(s). Subsequently, the organic solvent is evaporated from the oil-in-water-type emulsion to obtain a colloidal suspension of particles containing the slow release carrier and the compound of the present invention.

The pharmaceutical compositions herein will contain, per dosage unit, e.g., tablet, capsule, powder, injection, teaspoonful and the like, an amount of the active ingredient necessary to deliver an effective dose as described above. The pharmaceutical compositions herein will contain, per unit dosage unit, e.g., tablet, capsule, powder, injection, suppository, teaspoonful and the like, from about 0.01 mg to 200 mg/kg of body weight per day. Preferably, the range is from about 0.03 to about 100 mg/kg of body weight per day, most preferably, from about 0.05 to about 10 mg/kg of body weight per day. The compounds may be administered on a regimen of 1 to 5 times per day. The dosages, however, may be varied depending upon the requirement of the patients, the severity of the condition being treated and the compound being employed. The use of either daily administration or post-periodic dosing may be employed.

Preferably these compositions are in unit dosage forms such as tablets, pills, capsules, powders, granules, sterile parenteral solutions or suspensions, metered aerosol or liquid sprays, drops, ampoules, auto-injector devices or suppositories; for oral parenteral, intranasal, sublingual or rectal administration, or for administration by inhalation or insufflation. Alternatively, the composition may be presented in a form suitable for once-weekly or once-monthly administration; for example, an insoluble salt of the active compound, such as the decanoate salt, may be adapted to provide a depot preparation for intramuscular injection. For preparing solid compositions such as tablets, the principal active ingredient is mixed with a pharmaceutical carrier, e.g. conventional tableting ingredients such as corn starch, lactose, sucrose, sorbitol, talc, stearic acid, magnesium stearate, dicalcium phosphate or gums, and other pharmaceutical diluents, e.g. water, to form a solid preformulation composition containing a homogeneous mixture of a compound of the present invention, or a pharmaceutically acceptable salt thereof. When referring to these preformulation compositions as homogeneous, it is meant that the active ingredient is dispersed evenly throughout the composition so that the composition may be readily subdivided into equally effective dosage forms such as tablets, pills and capsules. This solid preformulation composition is then subdivided into unit dosage forms of the type described above containing from 0.1 to about 500 mg of the active ingredient of the present invention. The tablets or pills of the novel composition can be coated or otherwise compounded to provide a dosage form affording the advantage of prolonged action. For example, the tablet or pill can comprise an inner dosage and an outer dosage component, the latter being in the form of an envelope over the former. The two components can be separated by an enteric layer which serves to resist disintegration in the stomach and permits the inner component to pass intact into the duodenum or to be delayed in release. A variety of material can be used for such enteric layers or coatings, such materials including a number of polymeric acids with such materials as shellac, acetyl alcohol and cellulose acetate.

The liquid forms in which the compound of Formula I may be incorporated for administration orally or by injection include, aqueous solutions, suitably flavored syrups, aqueous or oil suspensions, and flavored emulsions with edible oils such as cottonseed oil, sesame oil, coconut oil or peanut oil, as well as elixirs and similar pharmaceutical vehicles. Suitable dispersing or suspending agents for aqueous suspensions, include synthetic and natural gums such as tragacanth, acacia, alginate, dextran, sodium carboxymethylcellulose, methylcellulose, polyvinyl-pyrrolidone or gelatin. The liquid forms in suitably flavored suspending or dispersing agents may also include the synthetic and natural gums, for example, tragacanth, acacia, methyl-cellulose and the like. For parenteral administration, sterile suspensions and solutions are desired. Isotonic preparations which generally contain suitable preservatives are employed when intravenous administration is desired.

Advantageously, compounds of Formula I may be administered in a single daily dose, or the total daily dosage may be administered in divided doses of two, three or four times daily. Furthermore, compounds for the present invention can be administered in intranasal form via topical use of suitable intranasal vehicles, or via transdermal skin patches well known to those of ordinary skill in that art. To be administered in the form of a transdermal delivery system, the dosage administration will, of course, be continuous rather than intermittent throughout the dosage regimen.

For instance, for oral administration in the form of a tablet or capsule, the active drug component can be combined with an oral, non-toxic pharmaceutically acceptable inert carrier such as ethanol, glycerol, water and the like. Moreover, when desired or necessary, suitable binders; lubricants, disintegrating agents and coloring agents can also be incorporated into the mixture. Suitable binders include, without limitation, starch, gelatin, natural sugars such as glucose or beta-lactose, corn sweeteners, natural and synthetic gums such as acacia, tragacanth or sodium oleate, sodium stearate, magnesium stearate, sodium benzoate, sodium acetate, sodium chloride and the like. Disintegrators include, without limitation, starch, methyl cellulose, agar, bentonite, xanthan gum and the like.

The daily dosage of the products of the present invention may be varied over a wide range from 1 to 5000 mg per adult human per day. For oral administration, the compositions are preferably provided in the form of tablets containing, 0.01, 0.05, 0.1, 0.5, 1.0, 2.5, 5.0, 10.0, 15.0, 25.0, 50.0, 100, 150, 200, 250 and 500 milligrams of the active ingredient for the symptomatic adjustment of the dosage to the patient to be treated. An effective amount of the drug is ordinarily supplied at a dosage level of from about 0.01 mg/kg to about 200 mg/kg of body weight per day. Particularly, the range is from about 0.03 to about 15 mg/kg of body weight per day, and more particularly, from about 0.05 to about 10 mg/kg of body weight per day. The compound of the present invention may be administered on a regimen up to four or more times per day, preferably of 1 to 2 times per day.

Optimal dosages to be administered may be readily determined by those skilled in the art, and will vary with the particular compound used, the mode of administration, the strength of the preparation, the mode of administration, and the advancement of the disease condition. In addition, factors associated with the particular patient being treated, including patient age, weight, diet and time of administration, will result in the need to adjust dosages.

The compounds of the present invention can also be administered in the form of liposome delivery systems, such as small unilamellar vesicles, large unilamellar vesicles, and multilamellar vesicles. Liposomes can be formed from a variety of lipids, including but not limited to amphipathic lipids such as phosphatidylcholines, sphingomyelins, phosphatidylethanolamines, phophatidylcholines, cardiolipins, phosphatidylserines, phosphatidylglycerols, phosphatidic acids, phosphatidylinositols, diacyl trimethylammonium propanes, diacyl dimethylammonium propanes, and stearylamine, neutral lipids such as triglycerides, and combinations thereof. They may either contain cholesterol or may be cholesterol-free.

The compounds of the present invention can also be administered locally. Any delivery device, such as intravascular drug delivery catheters, wires, pharmacological stents and endoluminal paving, may be utilized. The delivery system for such a device may comprise a local infusion catheter that delivers the compound at a rate controlled by the administer.

The present invention provides a drug delivery device comprising an intraluminal medical device, preferably a stent, and a therapeutic dosage of a compound of the invention.

The term “stent” refers to any device capable of being delivered by a catheter. A stent is routinely used to prevent vascular closure due to physical anomalies such as unwanted inward growth of vascular tissue due to surgical trauma. It often has a tubular, expanding lattice-type structure appropriate to be left inside the lumen of a duct to relieve an obstruction. The stent has a lumen wall-contacting surface and a lumen-exposed surface. The lumen-wall contacting surface is the outside surface of the tube and the lumen-exposed surface is the inner surface of the tube. The stent can be polymeric, metallic or polymeric and metallic, and it can optionally be biodegradable.

Methods of Treatment

The present invention includes methods of treating diseases in a mammal, including stroke.

The invention provides a method for reducing matrix metalloprotease activity in a cell comprising the step of contacting the cell with a compound of Formula I.

The invention provides a method for inhibiting matrix metalloprotease activity in a cell comprising the step of contacting the cell with a compound of Formula I.

The invention provides a method for reducing matrix metalloprotease activity in a subject comprising the step of administering a compound of Formula I to the subject.

The invention provides a method for inhibiting matrix metalloprotease activity in a subject comprising the step of administering a compound of Formula I to the subject.

The invention provides a method for preventing in a subject a disorder related to matrix metalloprotease activity comprising administering to the subject a prophylactically effective amount of a pharmaceutical composition comprising a compound of Formula I and a pharmaceutically acceptable carrier.

The invention provides a method of treating in a subject a disorder related to matrix metalloprotease activity comprising administering to the subject a therapeutically effective amount of a pharmaceutical composition comprising a compound of Formula I and a pharmaceutically acceptable carrier.

The invention provides a method for the treatment of stroke comprising administering to the subject a therapeutically effective amount of a pharmaceutical composition comprising a compound of Formula I and a pharmaceutically acceptable carrier.

While the foregoing specification teaches the principles of the present invention, with examples provided for the purpose of illustration, it will be understood that the practice of the invention encompasses all of the usual variations, adaptations and/or modifications as come within the scope of the following claims and their equivalents.

We claim:

1. Compounds of Formula I: 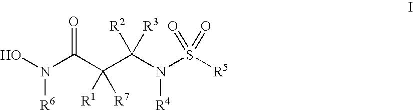
wherein:
R¹ is H, or —CH₃;
R² is H, or —CH₃;
R³ is H, C₍₁₋₃₎alkyl, phenyl, 5- or 6-membered heteroaryl;
wherein said phenyl and said 5- or 6-membered heteroaryl are optionally substituted with one or two substituents selected from the group consisting of Cl, F, —N(C₍₁₋₄₎alkyl)₂, —NO₂, —CN, —OCF₃, —CF₃, —OC₍₁₋₄₎alkyl, and C₍₁₋₄₎alkyl; 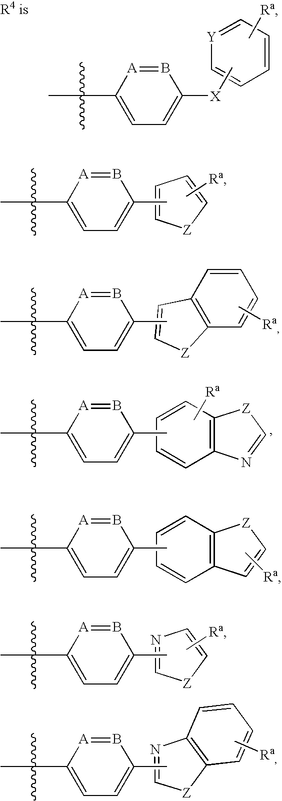 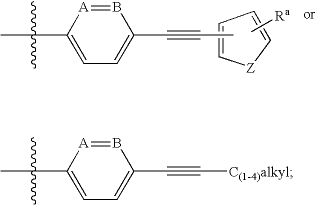
wherein:
A=B is —CH═CH— or S;
X is O, S, 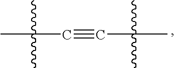
—CH═CH— (cis or trans), or a direct bond;
Y is CH or N;
Z is S or O or NH
R^{a} is one or two substituents independently selected from the group consisting of H, Cl, F, —OH, —NH₂, —N(C₍₁₋₄₎alkyl)₂, —NO₂, —CN, —CO₂H, —CONH₂, —OCF₃, —CF₃, —OC₍₁₋₄₎alkyl, and C₍₁₋₄₎alkyl;
R⁵ is C₍₁₋₄₎alkyl, phenyl, —C₍₁₋₃₎alkyl-phenyl, —C₍₁₋₃₎alkyl-heteroaryl, —N(C₍₁₋₄₎alkyl)₂, —C₍₁₋₃₎alkyl- 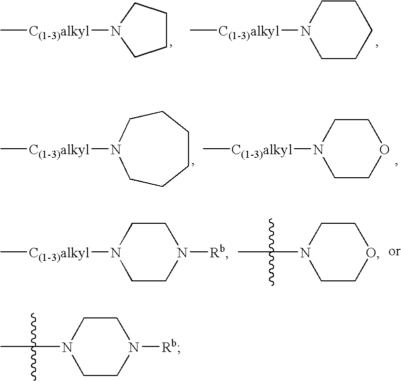
wherein said phenyl, —C₍₁₋₃₎alkyl-phenyl, and —C₍₁₋₃₎alkyl-heteroaryl are optionally substituted with one or two substituents selected from the group consisting of Cl, F, —N(C₍₁₋₄₎alkyl)₂, —NO₂, —CN, —CONH₂, —OCF₃, —CF₃, —OC₍₁₋₄₎alkyl, and C₍₁₋₄₎alkyl;
and R^{b} is —C₍₁₋₄₎alkyl, —SO₂C₍₁₋₄₎alkyl, —COC₍₁₋₄₎alkyl, or —CO₂CH₂-phenyl;
R⁶ is H, C₍₁₋₄₎alkyl, allyl, —C₍₂₋₄₎alkyl-O(C₍₁₋₄₎alkyl), —C₍₁₋₃₎alkyl-phenyl, —C₍₁₋₃₎alkyl-heteroaryl, —C₍₂₋₄₎alkyl-N(C₍₁₋₄₎alkyl)₂, 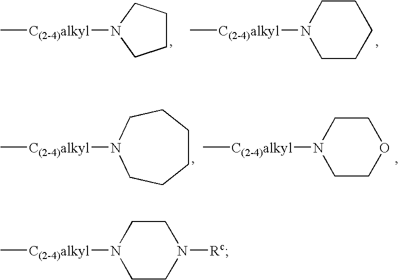
wherein said —C₍₁₋₃₎alkyl-phenyl and said —C₍₁₋₃₎alkyl-heteroaryl are optionally substituted with one or two substituents selected from the group consisting of Cl, F, —N(C₍₁₋₄₎alkyl)₂, —NO₂, —CN, —CONH₂, —OCF₃, —CF₃, —OC₍₁₋₄₎alkyl, and C₍₁₋₄₎alkyl;
wherein R^{c} is —C₍₁₋₄₎alkyl, —SO₂C₍₁₋₄₎alkyl, —COC₍₁₋₄₎alkyl, —CO₂CH₂-phenyl;
R⁷ is —OR⁸—SR⁸—NR⁹R¹⁰, or —NR¹¹COR¹²
wherein R⁸ is H, C₍₁₋₄₎alkyl, phenyl, heteroaryl, indolyl, C₍₁₋₂₎alkyl-phenyl, C₍₁₋₂₎alkyl-indolyl, or C₍₁₋₂₎alkyl-heteroaryl; wherein said phenyl, heteroaryl, indolyl, C₍₁₋₂₎alkyl-phenyl, C₍₁₋₂₎alkyl-indolyl, and C₍₁₋₂₎alkyl-heteroaryl are optionally substituted with one or two substituents selected from the group consisting of Cl, F, —N(C₍₁₋₄₎alkyl)₂, —NO₂, —CN, —CONH₂, —OCF₃, —CF₃, —OC₍₁₋₄₎alkyl, and C₍₁₋₄₎alkyl;
R⁹ is H, C₍₁₋₄₎alkyl, phenyl, heteroaryl, benzofused-heteroaryl, napthalenyl, C₍₃₋₇₎cycloalkyl, 5-7-membered heterocyclyl, C₍₂₋₄₎alkyl-carbamic acid benzyl ester, —C₍₂₋₄₎alkyl-1,5,5-trimethyl-imidazolidine-2,4-dione, C₍₁₋₃₎alkyl-phenyl, C₍₁₋₃₎alkyl-heteroaryl, C₍₁₋₃₎alkyl-benzofused-heteroaryl, and wherein said C₍₁₋₃₎alkyl-phenyl, C₍₁₋₃₎alkyl-heteroaryl, C₍₁₋₃₎alkyl-benzofused-heteroaryl, phenyl, heteroaryl, and said bezo-fused heteroaryl are optionally substituted with one or two substituents selected from the group consisting of Cl, F, —N(C₍₁₋₄₎alkyl)₂, —NO₂, —CN, —CONH₂, —OCF₃, —CF₃, —OC₍₁₋₄₎alkyl, and C₍₁₋₄₎alkyl;
R¹⁰ is H, or C₍₁₋₄₎alkyl; alternatively, R⁹ and R¹⁰ may join together with the attached nitrogen to form a ring selected from the group consisting of: 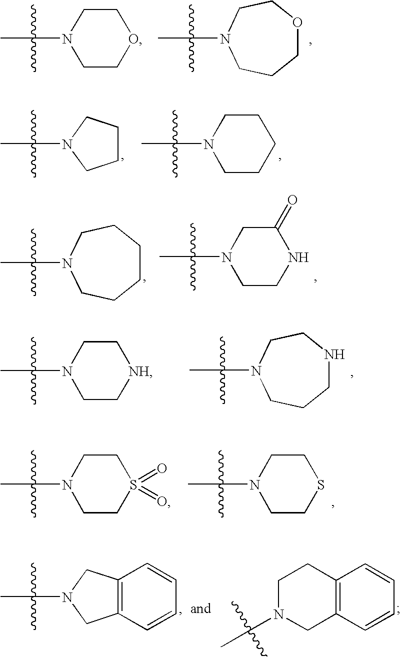
wherein said ring is optionally substituted with one or two substituents selected from the group consisting of: —CH₂OC₍₁₋₂₎alkyl, —N(C₍₁₋₄₎alkyl)₂, —OC₍₁₋₄₎alkyl, C₍₁₋₄₎alkyl, —OH, —SO₂C₍₁₋₄₎alkyl, —COC₍₁₋₄₎alkyl, —CONHC₍₁₋₄₎alkyl, phenyl, fluorophenyl, chlorophenyl, bromophenyl, and —CO₂CH₂phenyl.
R¹¹ is H, or C₍₁₋₄₎alkyl;
R¹² is C₍₁₋₄₎alkyl, indolyl, phenyl, heteroaryl, C₍₃₋₇₎cycloalkyl, morpholinyl, piperidinyl, piperazinyl, N-methyl piperazinyl, tetrahydropyranyl, pyrrolidinyl, C₍₁₋₂₎alkyl-phenyl, C₍₁₋₂₎alkyl-indolyl or C₍₁₋₂₎alkyl-heteroaryl; wherein said indolyl, phenyl, heteroaryl, C₍₁₋₂₎alkyl-phenyl, C₍₁₋₂₎alkyl-indolyl or C₍₁₋₂₎alkyl-heteroaryl are optionally substituted with one or two substituents selected from the group consisting of Cl, F, —N(C₍₁₋₄₎alkyl)₂, —NO₂, —CN, —CONH₂, —OCF₃, —CF₃, —OC₍₁₋₄₎alkyl, and C₍₁₋₄₎alkyl;
and solvates, hydrates, tautomers and pharmaceutically acceptable salts thereof.

2. A compound of claim 1 wherein
R³ is H, phenyl, or —CH₃;
wherein said phenyl is optionally substituted with one substituent selected from the group consisting of Cl, F, —N(C₍₁₋₄₎alkyl)₂, —OCF₃, —CF₃, —OC₍₁₋₄₎alkyl, and C₍₁₋₄₎alkyl; 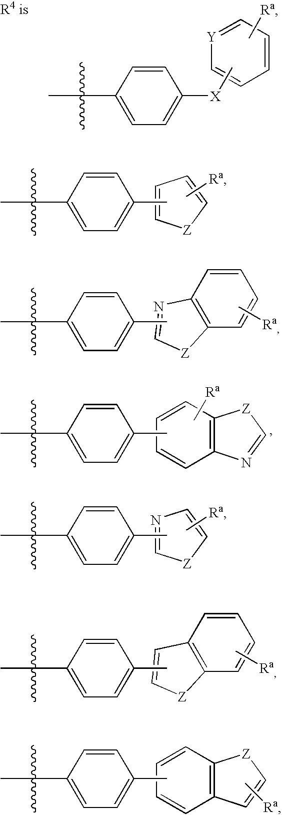 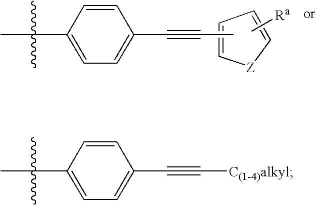
wherein:
X is O, S, 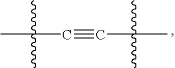
—CH═CH— (cis or trans), or a direct bond;
Y is CH or N;
Z is S or O or NH
R^{a} is one or two substituents independently selected from the group consisting of H, Cl, F, —OH, —NH₂, —N(C₍₁₋₄₎alkyl)₂, —NO₂, —CN, —CO₂H, —CONH₂, —OCF₃, —CF₃, —OC₍₁₋₄₎alkyl, and C₍₁₋₄₎alkyl;
R⁵ is C₍₁₋₄₎alkyl, phenyl, —C₍₁₋₃₎alkyl-phenyl, —C₍₁₋₃₎alkyl-heteroaryl, —N(C₍₁₋₄₎alkyl)₂, 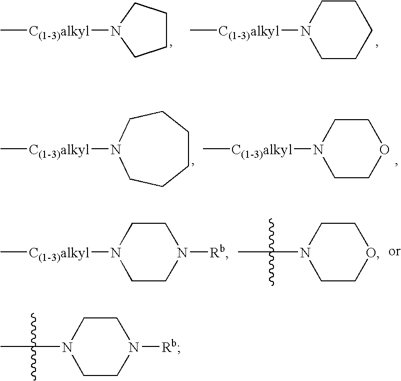
wherein said phenyl, —C₍₁₋₃₎alkyl-phenyl, and —C₍₁₋₃₎alkyl-heteroaryl are optionally substituted with one or two substituents selected from the group consisting of Cl, F, —N(C₍₁₋₄₎alkyl)₂, —OCF₃, —CF₃, —OC₍₁₋₄₎alkyl, and C₍₁₋₄₎alkyl;
and R^{b} is —C₍₁₋₄₎alkyl, —SO₂C₍₁₋₄₎alkyl, —COC₍₁₋₄₎alkyl, or —CO₂CH₂-phenyl;
R⁶ is H, C₍₁₋₄₎alkyl, allyl, —C₍₂₋₄₎alkyl-O(C₍₁₋₄₎alkyl), —C₍₁₋₃₎alkyl-phenyl, —C₍₁₋₃₎alkyl-heteroaryl, —C₍₂₋₄₎alkyl-N(C₍₁₋₄₎alkyl)₂, 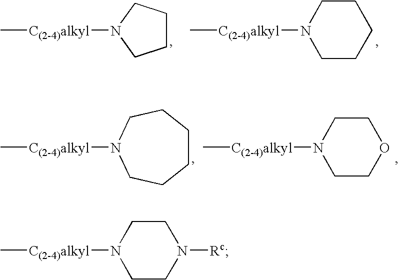
wherein said C₍₁₋₃₎alkyl-phenyl and said —C₍₁₋₃₎alkyl-heteroaryl are optionally substituted with one or two substituents selected from the group consisting of Cl, F, —N(C₍₁₋₄₎alkyl)₂, —OCF₃, —CF₃, —OC₍₁₋₄₎alkyl, and C₍₁₋₄₎alkyl;
wherein R^{c} is —C₍₁₋₄₎alkyl, —SO₂C₍₁₋₄₎alkyl, —COC₍₁₋₄₎alkyl, —CO₂CH₂-phenyl;
R⁷ is —OR⁸, —SR⁸—NR⁹R¹⁰, or —NR¹¹COR¹²
wherein R⁸ is H, C₍₁₋₄₎alkyl, phenyl, indolyl, pyridyl, C₍₁₋₂₎alkyl-phenyl, C₍₁₋₂₎alkyl-furanyl, C₍₁₋₂₎alkyl-pyrrolyl, C₍₁₋₂₎alkyl-indolyl, or C₍₁₋₂₎alkyl-pyridyl; wherein said phenyl, C₍₁₋₂₎alkyl-phenyl, C₍₁₋₂₎alkyl-furanyl, C₍₁₋₂₎alkyl-pyrrolyl, C₍₁₋₂₎alkyl-indolyl, and C₍₁₋₂₎alkyl-pyridyl are optionally substituted with one or two substituents selected from the group consisting of Cl, F, —OCF₃, —CF₃, —OC₍₁₋₄₎alkyl, and C₍₁₋₄₎alkyl;
R⁹ is H, C₍₁₋₄₎alkyl, phenyl, heteroaryl, benzofused-heteroaryl, napthalenyl, C₍₃₋₇₎cycloalkyl, C₍₂₋₄₎alkyl-carbamic acid benzyl ester, —C₍₂₋₄₎alkyl-1,5,5-trimethyl-imidazolidine-2,4-dione, C₍₁₋₃₎alkyl-phenyl, C₍₁₋₃₎alkyl-heteroaryl, C₍₁₋₃₎alkyl-benzofused-heteroaryl, and wherein said C₍₁₋₃₎alkyl-phenyl, C₍₁₋₃₎alkyl-heteroaryl, C₍₁₋₃₎alkyl-benzofused-heteroaryl, phenyl, heteroaryl, and said bezo-fused heteroaryl are optionally substituted with one or two substituents selected from the group consisting of Cl, F, —OCF₃, —CF₃, —OC₍₁₋₄₎alkyl, and C₍₁₋₄₎alkyl;
R¹⁰ is H, or C₍₁₋₄₎alkyl; alternatively, R⁹ and R¹⁰ may join together with the attached nitrogen to form a ring selected from the group consisting of: 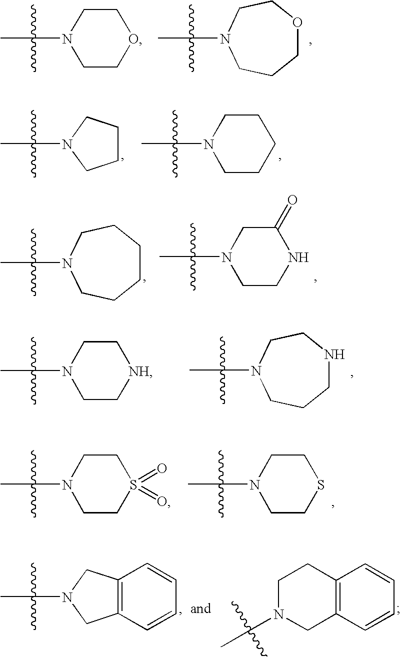
wherein said ring is optionally substituted with one or two substituents selected from the group consisting of: —CH₂OC₍₁₋₂₎alkyl, —N(C₍₁₋₄₎alkyl)₂, —OC₍₁₋₄₎alkyl, C₍₁₋₄₎alkyl, —OH, —SO₂C₍₁₋₄₎alkyl, —COC₍₁₋₄₎alkyl, —CONHC₍₁₋₄₎alkyl, phenyl, fluorophenyl, chlorophenyl, bromophenyl, and —CO₂CH₂phenyl.
R¹¹ is H, or C₍₁₋₄₎alkyl;
R¹² is C₍₁₋₄₎alkyl, indolyl, phenyl, heteroaryl, C₍₃₋₇₎cycloalkyl, morpholinyl, piperidinyl, piperazinyl, N-methyl piperazinyl, tetrahydropyranyl, pyrrolidinyl, C₍₁₋₂₎alkyl-phenyl, C₍₁₋₂₎alkyl-indolyl or C₍₁₋₂₎alkyl-heteroaryl; wherein said indolyl, phenyl, heteroaryl, C₍₁₋₂₎alkyl-phenyl, C₍₁₋₂₎alkyl-indolyl or C₍₁₋₂₎alkyl-heteroaryl are optionally substituted with one or two substituents selected from the group consisting of Cl, F, —N(C₍₁₋₄₎alkyl)₂, —OCF₃, —CF₃, —OC₍₁₋₄₎alkyl, and C₍₁₋₄₎alkyl;
and solvates, hydrates, tautomers and pharmaceutically acceptable salts thereof.

3. A compound of claim 2 wherein
R³ is H, or —CH₃; 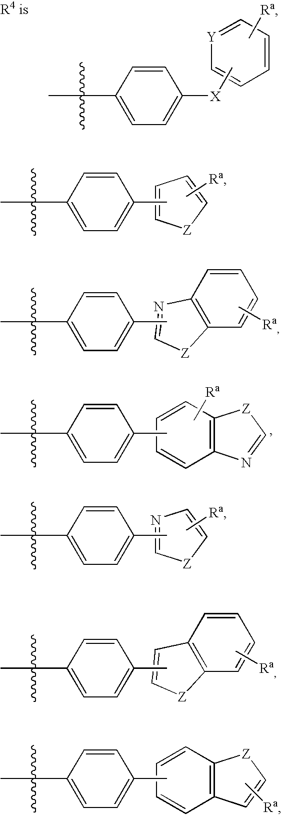 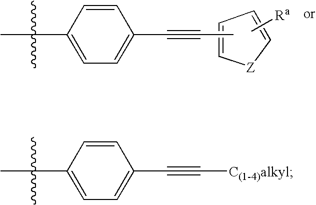
wherein:
X is O, S, 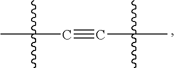
—CH═CH— (cis or trans), or a direct bond;
Y is CH or N;
Z is S or O or NH
R^{a} is one substituent selected from the group consisting of H, Cl, F, —OH, —NH₂, —N(C₍₁₋₄₎alkyl)₂, —NO₂, —CN, —CO₂H, —CONH₂, —OCF₃, —CF₃, —OC₍₁₋₄₎alkyl, and C₍₁₋₄₎alkyl;
R⁵ is C₍₁₋₄₎alkyl, phenyl, —C₍₁₋₃₎alkyl-phenyl, —C₍₁₋₃₎alkyl-heteroaryl, —N(C₍₁₋₄₎alkyl)₂, 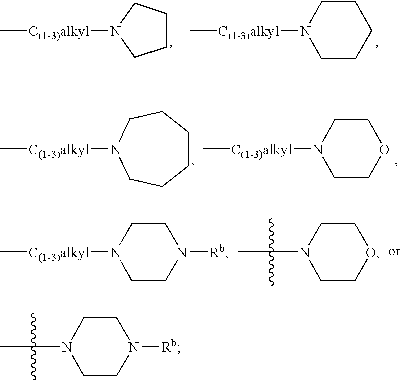
wherein said C₍₁₋₃₎alkyl-phenyl is optionally substituted with one substituent selected from the group consisting of Cl, F, —OCF₃, —CF₃, —OC₍₁₋₄₎alkyl, and C₍₁₋₄₎alkyl;
wherein R^{b} is —C₍₁₋₄₎alkyl, —SO₂C₍₁₋₄₎alkyl, —COC₍₁₋₄₎alkyl, or —CO₂CH₂-phenyl;
R⁶ is H, C₍₁₋₄₎alkyl, allyl, —C₍₂₋₄₎alkyl-O(C₍₁₋₄₎alkyl), —C₍₁₋₃₎alkyl-phenyl, —C₍₁₋₃₎alkyl-heteroaryl, 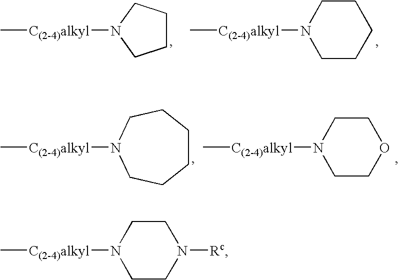
or —C₍₂₋₄₎alkyl-N(C₍₁₋₄₎alkyl)₂;
wherein said C₍₁₋₃₎alkyl-phenyl is optionally substituted with one substituent selected from the group consisting of Cl, F, —OCF₃, —CF₃, —OC₍₁₋₄₎alkyl, and C₍₁₋₄₎alkyl;
wherein R^{c} is —C₍₁₋₄₎alkyl, —SO₂C₍₁₋₄₎alkyl, —COC₍₁₋₄₎alkyl, —CO₂CH₂-phenyl;
R⁷ is —OR⁸, —SR⁸—NR⁹R¹⁰, or —NR¹¹COR¹²;
wherein R⁸ is H, C₍₁₋₄₎alkyl, phenyl, indolyl, pyridyl, C₍₁₋₂₎alkyl-phenyl, C₍₁₋₂₎alkyl-indolyl, or C₍₁₋₂₎alkyl-pyridyl; wherein said phenyl, C₍₁₋₂₎alkyl-phenyl, C₍₁₋₂₎alkyl-indolyl, and C₍₁₋₂₎alkyl-pyridyl are optionally substituted with one or two substituents selected from the group consisting of Cl, F, —OCF₃, —CF₃, —OC₍₁₋₄₎alkyl, and C₍₁₋₄₎alkyl;
R⁹ is H, C₍₁₋₄₎alkyl, phenyl, heteroaryl, benzofused-heteroaryl, napthalenyl, C₍₃₋₇₎cycloalkyl, C₍₂₋₄₎alkyl-carbamic acid benzyl ester, —C₍₂₋₄₎alkyl-1,5,5-trimethyl-imidazolidine-2,4-dione, C₍₁₋₃₎alkyl-phenyl, C₍₁₋₃₎alkyl-heteroaryl, C₍₁₋₃₎alkyl-benzofused-heteroaryl, and wherein said C₍₁₋₃₎alkyl-phenyl, C₍₁₋₃₎alkyl-heteroaryl, C₍₁₋₃₎alkyl-benzofused-heteroaryl, phenyl, heteroaryl, and said bezo-fused heteroaryl are optionally substituted with one or two substituents selected from the group consisting of Cl, F, —OCF₃, —CF₃, —OC₍₁₋₄₎alkyl, and C₍₁₋₄₎alkyl;
R¹⁰ is H, or C₍₁₋₄₎alkyl; alternatively, R⁹ and R¹⁰ may join together with the attached nitrogen to form a ring selected from the group consisting of: 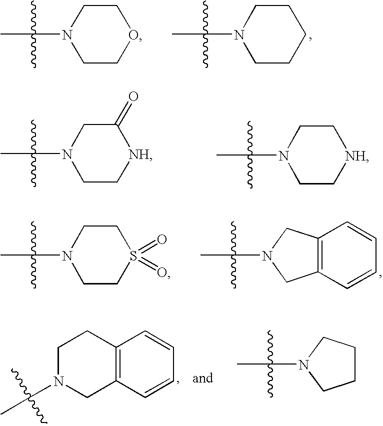
wherein said ring is optionally substituted with one or two substituents selected from the group consisting of: —CH₂OC₍₁₋₂₎alkyl, —N(C₍₁₋₃₎alkyl)₂, —OC₍₁₋₃₎alkyl, C₍₁₋₃₎alkyl, —OH, —SO₂CH₃, —COC₍₁₋₃₎alkyl, —CONHC₍₁₋₃₎alkyl, phenyl, chlorophenyl, bromophenyl, and —CO₂CH₂phenyl.
R¹¹ is H, or C₍₁₋₄₎alkyl;
R¹² is C₍₁₋₄₎alkyl, indolyl, phenyl, heteroaryl, C₍₅₋₆₎cycloalkyl, morpholinyl, piperidinyl, piperazinyl, N-methyl piperazinyl, tetrahydropyranyl, pyrrolidinyl; wherein said indolyl, phenyl, or heteroaryl, are optionally substituted with one or two substituents selected from the group consisting of Cl, F, —N(C₍₁₋₄₎alkyl)₂, —OCF₃, —CF₃, —OC₍₁₋₄₎alkyl, and C₍₁₋₄₎alkyl;
and solvates, hydrates, tautomers and pharmaceutically acceptable salts thereof.

4. A compound of claim 3 wherein 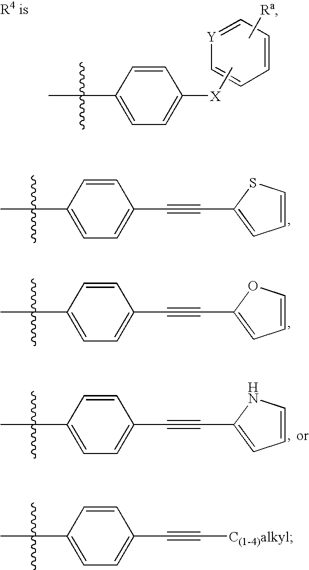
wherein:
X is O, S, 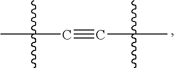
—CH═CH— (cis or trans), or a direct bond;
Y is CH or N;
R^{a} is one substituent selected from the group consisting of H, Cl, F, —OH, —NH₂, —N(C₍₁₋₄₎alkyl)₂, —NO₂, —CN, —CO₂H, —CONH₂, —OCF₃, —CF₃, —OC₍₁₋₄₎alkyl, and C₍₁₋₄₎alkyl;
R⁵ is C₍₁₋₄₎alkyl, phenyl, —N(CH₃)₂, —C₍₁₋₃₎alkyl-phenyl, or —C₍₁₋₃₎alkyl-pyridyl;
R⁶ is H, C₍₁₋₄₎alkyl, allyl, —C₍₁₋₃₎alkyl-phenyl, or 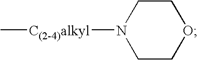
R⁷ is —OR⁸, —SR⁸—NR⁹R¹⁰, or —NR¹¹COR¹²;
wherein R³ is H, C₍₁₋₄₎alkyl, phenyl, C₍₁₋₂₎alkyl-phenyl; wherein said phenyl, and C₍₁₋₂₎alkyl-phenyl, are optionally substituted with one or two substituents selected from the group consisting of Cl, F, —OCF₃, —CF₃, —OC₍₁₋₄₎alkyl, and C₍₁₋₄₎alkyl;
R⁹ is H, C₍₁₋₄₎alkyl, phenyl, fluorophenyl, difluorophenyl, fluorochlorophenyl, chlorophenyl, trifluoromethylphenyl, dimethoxyphenyl, methoxyphenyl, trifluoromethoxyphenyl, napthalenyl, cyclopentyl, indolyl, C₍₁₋₂₎alkyl-indolyl, 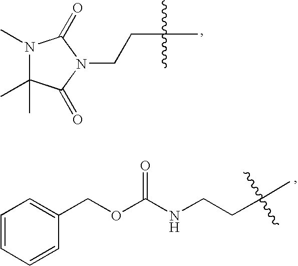
C₍₁₋₂₎alkyl-pyridyl, or C₍₁₋₂₎alkyl-phenyl;
R¹⁰ is H, or C₍₁₋₄₎alkyl; alternatively, R⁹ and R¹⁰ may join together with the attached nitrogen to form a ring selected from the group consisting of: 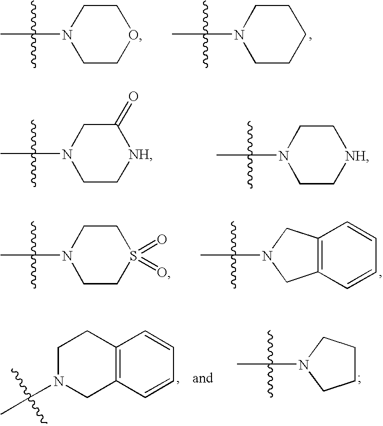
wherein said ring is optionally substituted with one or two substituents selected from the group consisting of: —CH₂OC₍₁₋₂₎alkyl, —N(C₍₁₋₃₎alkyl)₂, —OC₍₁₋₃₎alkyl, C₍₁₋₃₎alkyl, —OH, —SO₂CH₃, —COC₍₁₋₃₎alkyl, —CONHC₍₁₋₃₎alkyl, phenyl, chlorophenyl, bromophenyl, and —CO₂CH₂phenyl.
R¹¹ is H, or C₍₁₋₄₎alkyl;
R¹² is C₍₁₋₄₎alkyl, indolyl, phenyl, C₍₅₋₆₎cycloalkyl, tetrahydropyranyl;
wherein said indolyl, or phenyl are optionally substituted with one substituent selected from the group consisting of Cl, F, —N(C₍₁₋₄₎alkyl)₂, —OCF₃, —CF₃, —OC₍₁₋₄₎alkyl, and C₍₁₋₄₎alkyl;
and solvates, hydrates, tautomers and pharmaceutically acceptable salts thereof.

5. A compound of claim 4 wherein
R² is H;
R³ is H;
R⁴ is 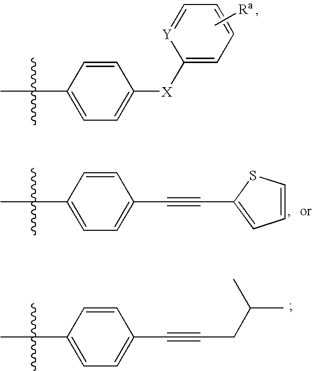
wherein:
X is O, S, 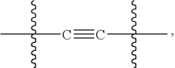
or a direct bond;
Y is CH or N;
R^{a} is H, Cl, —CF₃, —OCH₃, or —CH₃;
R⁵ is C₍₁₋₃₎alkyl, phenyl, —N(CH₃)₂, 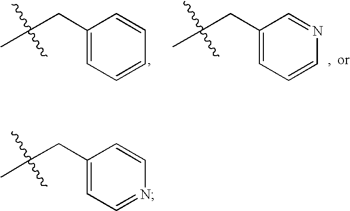
R⁶ is H, C₍₁₋₄₎alkyl, allyl, or 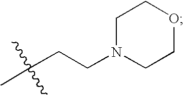
R⁷ is —OR⁸, —NR⁹R¹⁰, or —NR¹¹COR¹²;
wherein R⁸ is H, C₍₁₋₃₎alkyl, or 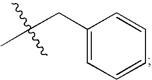
R⁹ is H, C₍₁₋₄₎alkyl, phenyl, fluorophenyl, difluorophenyl, fluorochlorophenyl, chlorophenyl, trifluoromethylphenyl, methoxyphenyl, trifluoromethoxyphenyl, napthalenyl, cyclopentyl, 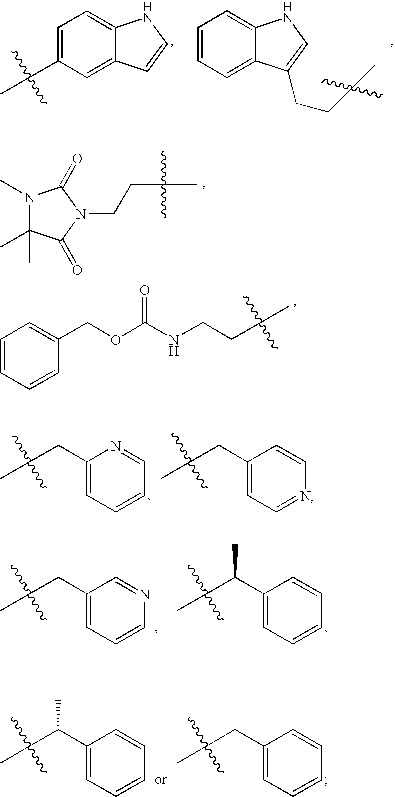
R¹⁰ is H, or C₍₁₋₃₎alkyl; alternatively, R⁹ and R¹⁰ may join together with the attached nitrogen to form a ring selected from the group consisting of: 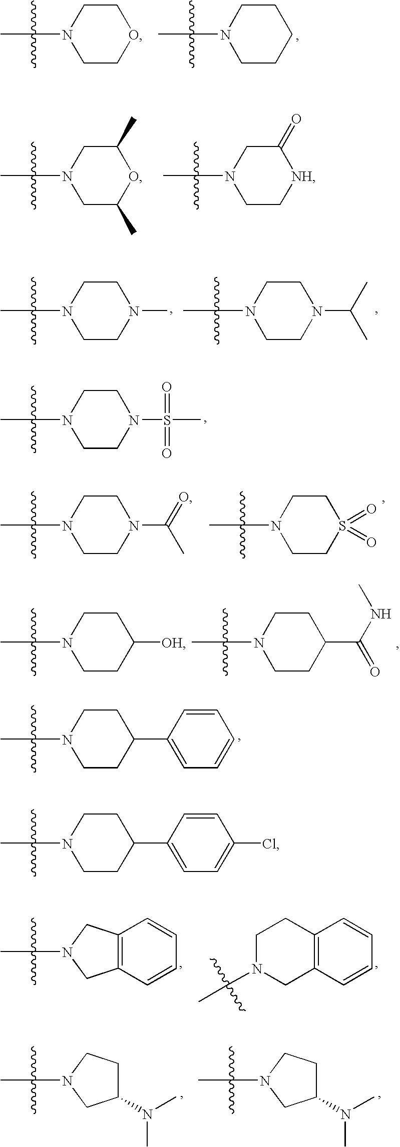 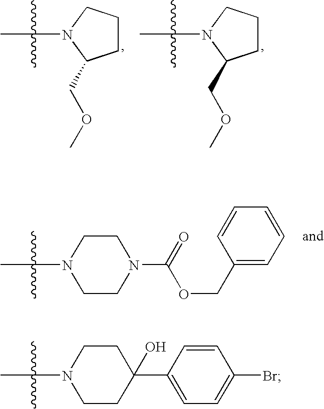
R¹¹ is H, or C₍₁₋₄₎alkyl;
R¹² is 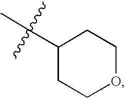
or C₍₁₋₄₎alkyl;
and solvates, hydrates, tautomers and pharmaceutically acceptable salts thereof.

6. A compound selected from the group consisting of

| Cpd. | | |
|---|---|---|
| # | Structure | Name |
| 1 | 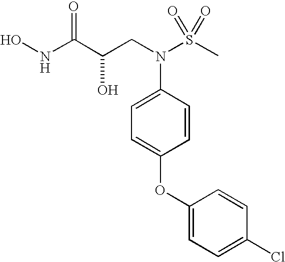 | N-Hydroxy (2S)-2-hydroxy-3-{N-[4-(4- chlorophenoxy)phenyl], N- methanesulfonylamino}propionamide |
| 2 | 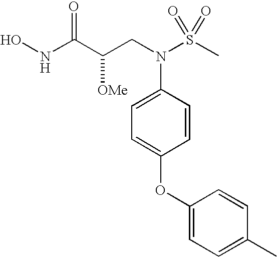 | N-Hydroxy (2S)-2-methoxy-3-{N-[4-(4- methylphenoxy)phenyl], N- methanesulfonylamino}propionamide |
| 3 | 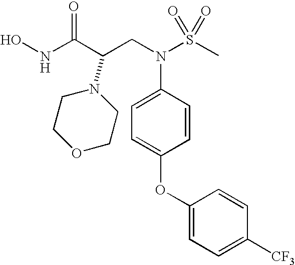 | N-Hydroxy (2S)-2-(morpholin-4-yl)-3-{N-[4- (4-trifluoromethylphenoxy)phenyl], N- methanesulfonylamino}propionamide |
| 4 | 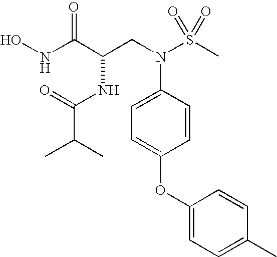 | N-Hydroxy (2S)-3-{N-[4-(4- methylphenoxy)phenyl], N- methanesulfonylamino}-2-(2- propylcarbonylamino)propionamide |
| 5 | 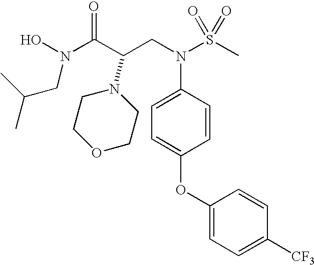 | N-Hydroxy, N-(2-methylpropyl) (2S)-2- (morpholin-4-yl)-3-{N-[4-(4- trifluoromethylphenoxy)phenyl], N- methanesulfonylamino}propionamide |
| 6 | 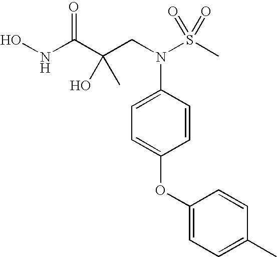 | N-Hydroxy 2-hydroxy-2-methyl-3-{N-[4-(4- methylphenoxy)phenyl], N- methanesulfonylamino}propionamide |
| 7 | 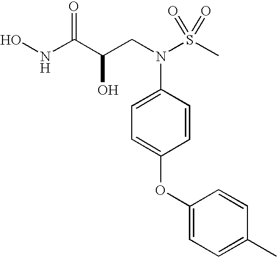 | N-Hydroxy (2R)-2-hydroxy-3-{N-[4-(4- methylphenoxy)phenyl], N- methanesulfonylamino}propionamide |
| 8 | 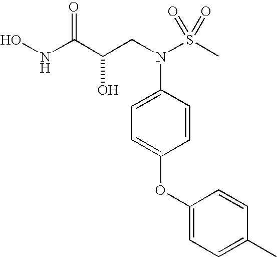 | N-Hydroxy (2S)-2-hydroxy-3-{N-[4-(4- methylphenoxy)phenyl], N- methanesulfonylamino}propionamide |
| 9 | 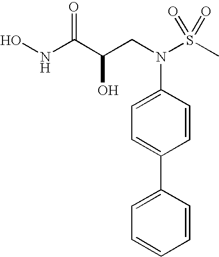 | N-Hydroxy (2R)-2-hydroxy-3-[N-(4- phenyl)phenyl, N- methanesulfonylamino]propionamide |
| 10 | 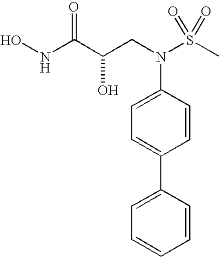 | N-Hydroxy (2S)-2-hydroxy-3-[N-(4- phenyl)phenyl, N- methanesulfonylamino]propionamide |
| 11 | 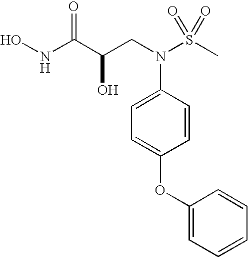 | N-Hydroxy (2R)-2-hydroxy-3-[N-(4- phenoxy)phenyl, N- methanesulfonylamino]propionamide |
| 12 | 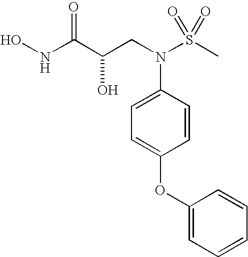 | N-Hydroxy (2S)-2-hydroxy-3-[N-(4- phenoxy)phenyl, N- methanesulfonylamino]propionamide |
| 13 | 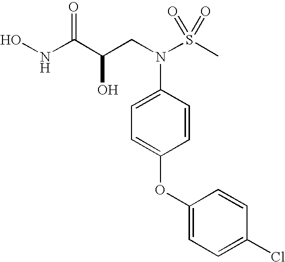 | N-Hydroxy (2R)-2-hydroxy-3-{N-[4-(4- chlorophenoxy)phenyl], N- methanesulfonylamino}propionamide |
| 14 | 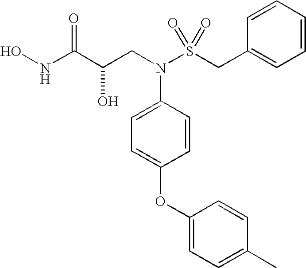 | N-Hydroxy (2S)-2-hydroxy-3-{N-[4-(4- methylphenoxy)phenyl], N- phenylmethylsulfonylamino}propionamide |
| 15 | 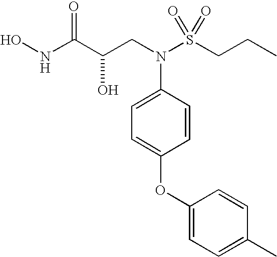 | N-Hydroxy (2S)-2-hydroxy-3-{N-[4-(4- methylphenoxy)phenyl], N-(1- propyl)sulfonylamino}propionamide |
| 16 | 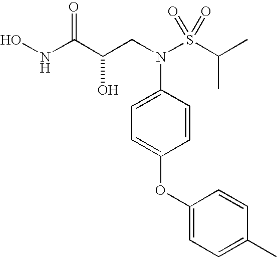 | N-Hydroxy (2S)-2-hydroxy-3-{N-[4-(4- methylphenoxy)phenyl], N-(2- propyl)sulfonylamino}propionamide |
| 17 | 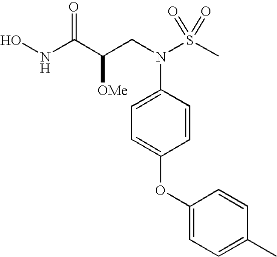 | N-Hydroxy (2R)-2-methoxy-3-{N-[4-(4- methylphenoxy)phenyl], N- methanesulfonylamino}propionamide |
| 18 | 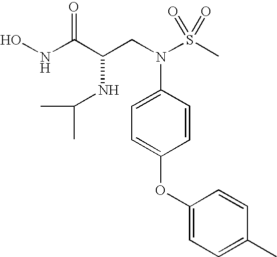 | N-Hydroxy (2S)-3-{N-[4-(4- methylphenoxy)phenyl], N- methanesulfonylamino}-2-[(2- propyl)amino]propionamide |
| 19 | 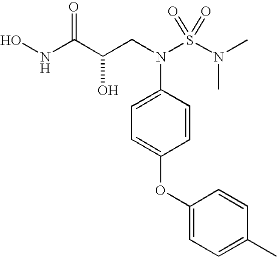 | N-Hydroxy (2S)-2-hydroxy-3-{N-[4-(4- methylphenoxy)phenyl], N- (dimethylamino)sulfonylamino}propionamide |
| 20 | 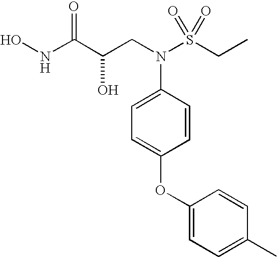 | N-Hydroxy (2S)-2-hydroxy-3-{N-[4-(4- methylphenoxy)phenyl], N- ethylsulfonylamino}propionamide |
| 21 | 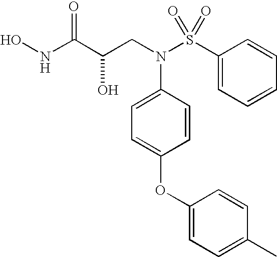 | N-Hydroxy (2S)-2-hydroxy-3-{N-[4-(4- methylphenoxy)phenyl], N- phenylsulfonylamino}propionamide |
| 22 | 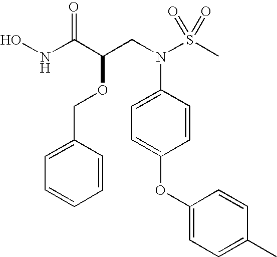 | N-Hydroxy (2R)-2-benzyloxy-3-{N-[4-(4- methylphenoxy)phenyl], N- methanesulfonylamino}propionamide |
| 23 | 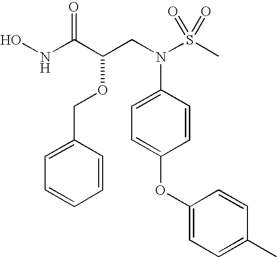 | N-Hydroxy (2S)-2-benzyloxy-3-{N-[4-(4- methylphenoxy)phenyl], N- methanesulfonylamino}propionamide |
| 24 | 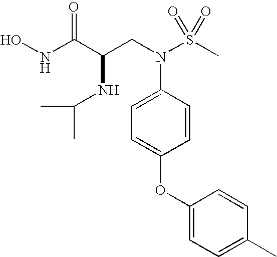 | N-Hydroxy (2R)-3-{N-[4-(4- methylphenoxy)phenyl], N- methanesulfonylamino}-2-(2- propyl)aminopropionamide |
| 25 | 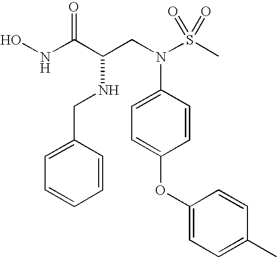 | N-Hydroxy (2S)-2-benzylamino-3-{N-[4-(4- methylphenoxy)phenyl], N- methanesulfonylamino}propionamide |
| 26 | 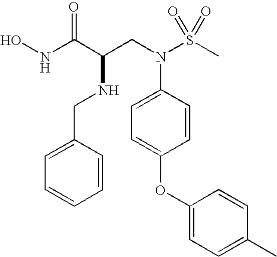 | N-Hydroxy (2R)-2-benzylamino-3-{N-[4-(4- methylphenoxy)phenyl], N- methanesulfonylamino}propionamide |
| 27 | 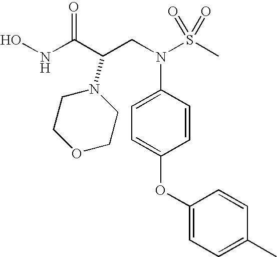 | N-Hydroxy (2S)-3-{N-[4-(4- methylphenoxy)phenyl], N- methanesulfonylamino}-2-(morpholin-4- yl)propionamide |
| 28 | 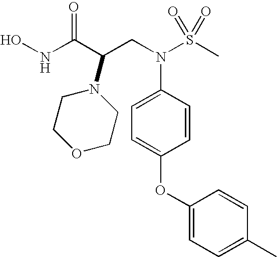 | N-Hydroxy (2R)-3-{N-[4-(4- methylphenoxy)phenyl], N- methanesulfonylamino}-2-(morpholin-4- yl)propionamide |
| 29 | 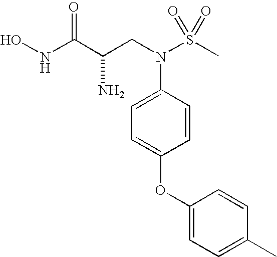 | N-Hydroxy (2S)-2-amino-3-{N-[4-(4- methylphenoxy)phenyl], N- methanesulfonylamino}propionamide |
| 30 | 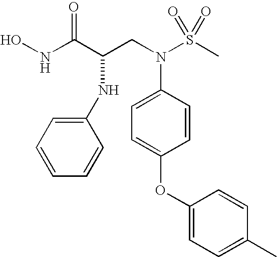 | N-Hydroxy (2S)-3-{N-[4-(4- methylphenoxy)phenyl], N- methanesulfonylamino}-2- phenylaminopropionamide |
| 31 | 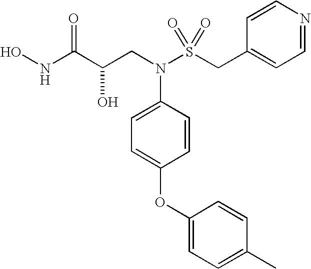 | N-Hydroxy (2S)-2-hydroxy-3-{N-[4-(4- methylphenoxy)phenyl], N-(pryidin-4- yl)methylsulfonylamino}propionamide |
| 32 | 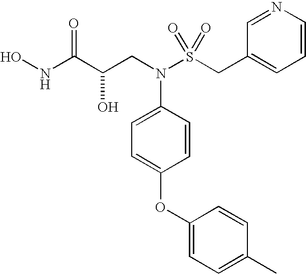 | N-Hydroxy (2S)-2-hydroxy-3-{N-[4-(4- methylphenoxy)phenyl], N-(pryidin-3- yl)methylsulfonylamino}propionamide |
| 33 | 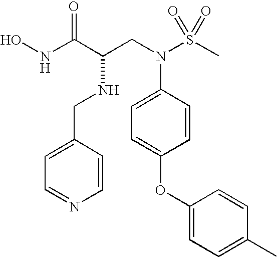 | N-Hydroxy (2S)-3-{N-[4-(4- methylphenoxy)phenyl], N- methanesulfonylamino}-2-(pyridine-4- yl)methylaminopropionamide |
| 34 | 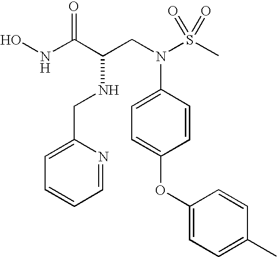 | N-Hydroxy (2S)-3-{N-[4-(4- methylphenoxy)phenyl], N- methanesulfonylamino}-2-(pyridin2- yl)methylaminopropionamide |
| 35 | 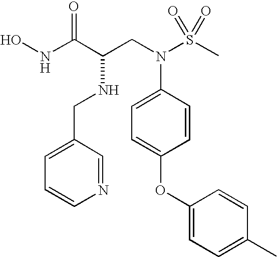 | N-Hydroxy (2S)-3-{N-[4-(4- methylphenoxy)phenyl], N- methanesulfonylamino}-2-(pyridine-3- yl)methylaminopropionamide |
| 36 | 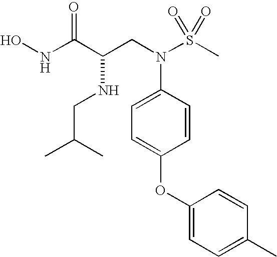 | N-Hydroxy (2S)-3-{N-[4-(4- methylphenoxy)phenyl], N- methanesulfonylamino}-2-(2- methylpropyl)aminopropionamide |
| 37 | 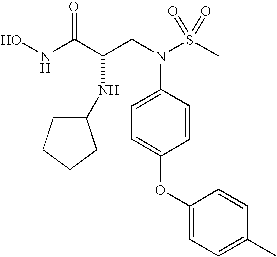 | N-Hydroxy (2S)-2-cyclopentylamino-3-{N-[4- (4-methylphenoxy)phenyl], N- methanesulfonylamino}propionamide |
| 38 | 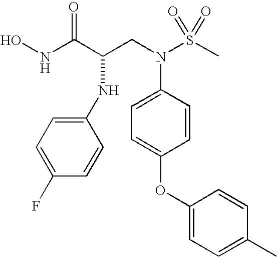 | N-Hydroxy (2S)-2-(4-fluorophenyl)amino-3- {N-[4-(4-methylphenoxy)phenyl], N- methanesulfonylamino}propionamide |
| 39 | 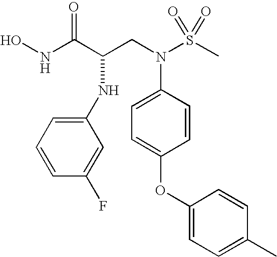 | N-Hydroxy (2S)-2-(3-fluorophenyl)amino-3- {N-[4-(4-methylphenoxy)phenyl], N- methanesulfonylamino}propionamide |
| 40 | 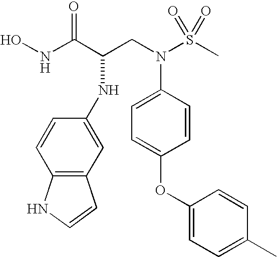 | N-Hydroxy (2S)-2-(indol-5-yl)amino-3-{N-[4- (4-methylphenoxy)phenyl], N- methanesulfonylamino}propionamide |
| 41 | 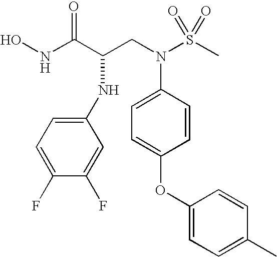 | N-Hydroxy (2S)-2-(3,4-difluorophenyl)amino- 3-{N-[4-(4-methylphenoxy)phenyl], N- methanesulfonylamino}propionamide |
| 42 | 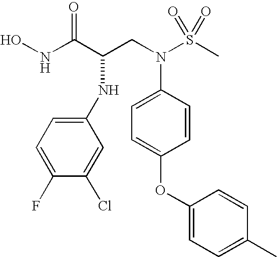 | N-Hydroxy (2S)-2-(3-chloro-4- fluorophenyl)amino-3-{N-[4-(4- methylphenoxy)phenyl], N- methanesulfonylamino}propionamide |
| 43 | 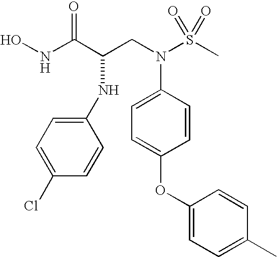 | N-Hydroxy (2S)-2-(4-chlorophenyl)amino-3- {N-[4-(4-methylphenoxy)phenyl], N- methanesulfonylamino}propionamide |
| 44 | 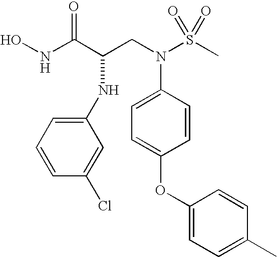 | N-Hydroxy (2S)-2-(3-chlorophenyl)amino-3- {N-[4-(4-methylphenoxy)phenyl], N- methanesulfonylamino}propionamide |
| 45 | 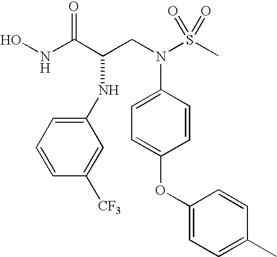 | N-Hydroxy (2S)-3-{N-[4-(4- methylphenoxy)phenyl], N- methanesulfonylamino}-2-(3- trifluoromethylphenyl)aminopropionamide |
| 46 | 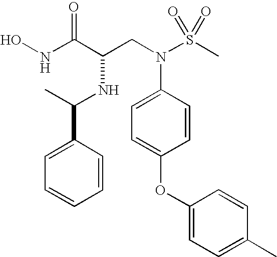 | N-Hydroxy (2S)-3-{N-[4-(4- methylphenoxy)phenyl], N- methanesulfonylamino}-2-[(1R)-1-phenyl-1- ethyl]aminopropionamide |
| 47 | 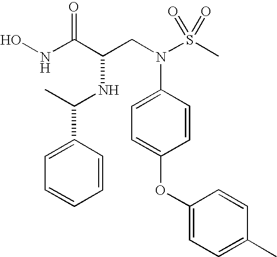 | N-Hydroxy (2S)-3-{N-[4-(4- methylphenoxy)phenyl], N- methanesulfonylamino}-2-[(1S)-1-phenyl-1- ethyl]aminopropionamide |
| 48 | 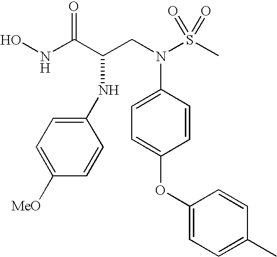 | N-Hydroxy (2S)-2-(4-methoxyphenyl)amino- 3-{N-[4-(4-methylphenoxy)phenyl], N- methanesulfonylamino}propionamide |
| 49 | 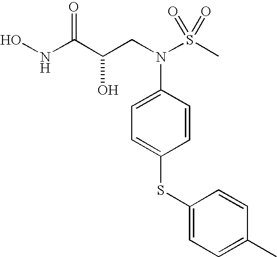 | N-Hydroxy (2S)-2-hydroxy-3-{N-[4-(4- methylthiophenoxy)phenyl], N- methanesulfonylamino}propionamide |
| 50 | 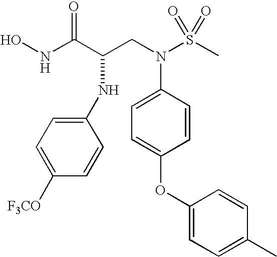 | N-Hydroxy (2S)-3-{N-[4-(4- methylphenoxy)phenyl], N- methanesulfonylamino}-2-(4- trifluoromethoxyphenyl)aminopropionamide |
| 51 | 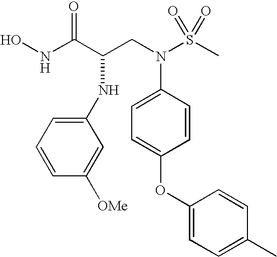 | N-Hydroxy (2S)-2-(3-methoxyphenyl)amino- 3-{N-[4-(4-methylphenoxy)phenyl], N- methanesulfonylamino}propionamide |
| 52 | 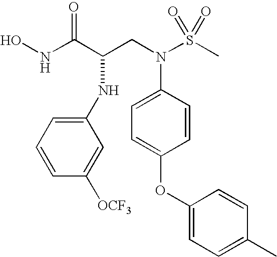 | N-Hydroxy (2S)-3-{N-[4-(4- methylphenoxy)phenyl], N- methanesulfonylamino}-2-(3- trifluoromethoxyphenyl)aminopropionamide |
| 53 | 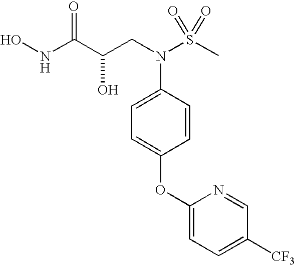 | N-Hydroxy (2S)-2-hydroxy-3-{N-{4-[(5- trifluoromethylpyridin-2-yl)oxy]phenyl}, N- methanesulfonylamino}propionamide |
| 54 | 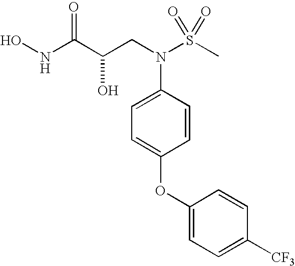 | N-Hydroxy (2S)-2-hydroxy-3-{N-[4-(4- trifluoromethylphenoxy)phenyl], N- methanesulfonylamino}propionamide |
| 55 | 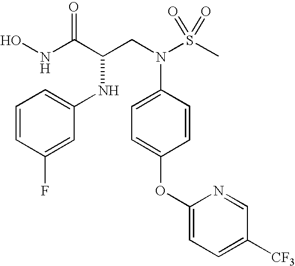 | N-Hydroxy (2S)-2-(3-fluorophenyl)amino-3- {N-{4-[(5-trifluoromethylpyridin-2- yl)oxy]phenyl}, N- methanesulfonylamino}propionamide |
| 56 | 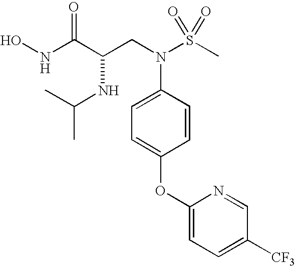 | N-Hydroxy (2S)-2-(2-propyl)amino-3-{N-{4- [(5-trifluoromethylpyridin-2-yl)oxy]phenyl}, N- methanesulfonylamino}propionamide |
| 57 | 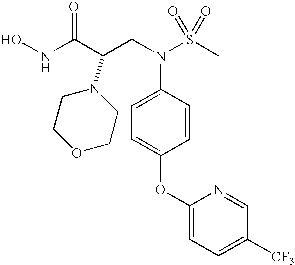 | N-Hydroxy (2S)-2-(morpholin-4-yl)-3-{N-{4- [(5-trifluoromethylpyridin-2-yl)oxy]phenyl}, N- methanesulfonylamino}propionamide |
| 58 | 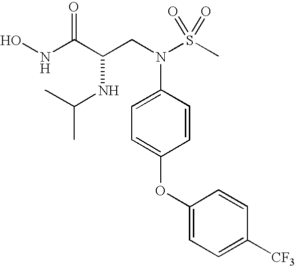 | N-Hydroxy (2S)-2-(2-propyl)amino-3-{N-[4- (4-trifluoromethylphenoxy)phenyl], N- methanesulfonylamino}propionamide |
| 59 | 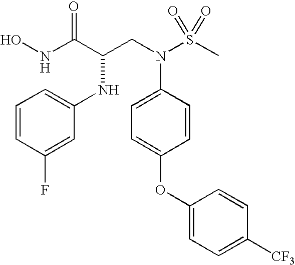 | N-Hydroxy (2S)-2-(3-fluorophenyl)amino-3- {N-[4-(4-trifluoromethylphenoxy)phenyl], N- methanesulfonylamino}propionamide |
| 60 | 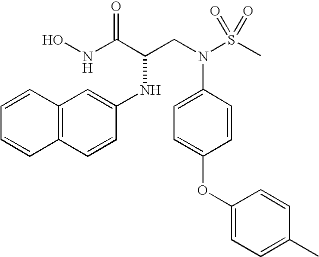 | N-Hydroxy (2S)-3-{N-[4-(4- methylphenoxy)phenyl], N- methanesulfonylamino}-2-(naphthylen-2- yl)aminopropionamide |
| 61 | 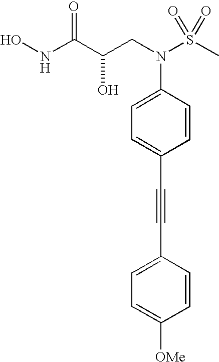 | N-Hydroxy (2S)-2-hydroxy-3-{N-[4-(4- methoxyphenylacetylene)phenyl], N- methanesulfonylamino}propionamide |
| 62 | 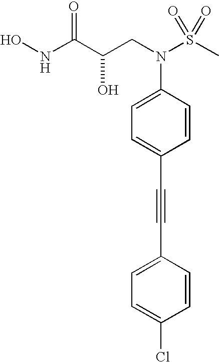 | N-Hydroxy (2S)-2-hydroxy-3-{N-[4-(4- chlorophenylacetylene)phenyl], N- methanesulfonylamino}propionamide |
| 63 | 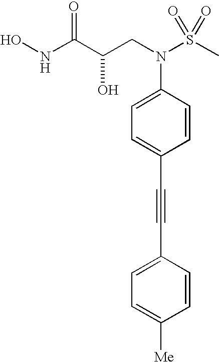 | N-Hydroxy (2S)-2-hydroxy-3-{N-[4-(4- methylphenylacetylene)phenyl], N- methanesulfonylamino}propionamide |
| 64 | 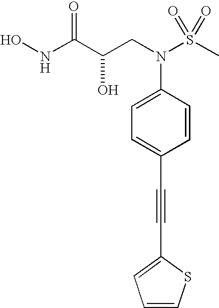 | N-Hydroxy (2S)-2-hydroxy-3-{N-[4-(4- thiophen-2-ylacetylene)phenyl], N- methanesulfonylamino}propionamide |
| 65 | 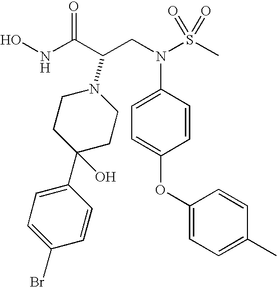 | N-Hydroxy (2S)-2-[4-(4-bromophenyl)-4- hydroxypiperidin-1-yl]-3-{N-[4-(4- methylphenoxy)phenyl], N- methanesulfonylamino}propionamide |
| 66 | 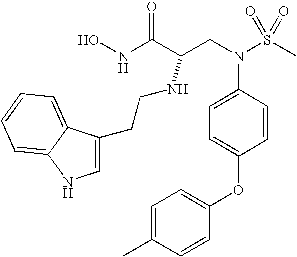 | N-Hydroxy (2S)-2-[(2-(indol-3-yl)ethylamino]- 3-{N-[4-(4-methylphenoxy)phenyl], N- methanesulfonylamino}propionamide |
| 67 | 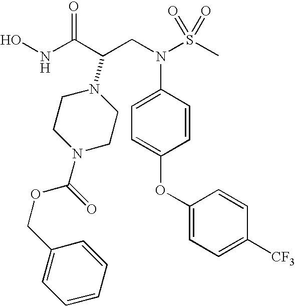 | N-Hydroxy (2S)-2-[(4- benzyloxycarbonyl)piperazin-1-yl]-3-{N-[4-(4- trifluoromethylphenoxy)phenyl], N- methanesulfonylamino}propionamide |
| 68 | 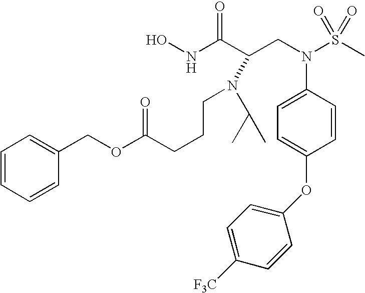 | N-Hydroxy (2S)-2-{N-(2-propyl), N-[2- benzyloxycarbonylamino)ethyl]amino}-3-{N- [4-(4-trifluoromethylphenoxy)phenyl], N- methanesulfonylamino}propionamide |
| 69 | 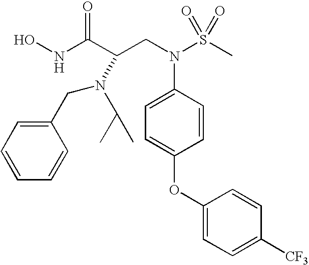 | N-Hydroxy (2S)-2-[N-benzyl-N-(2- propyl)amino]-3-{N-[4-(4- trifluoromethylphenoxy)phenyl], N- methanesulfonylamino}propionamide |
| 70 | 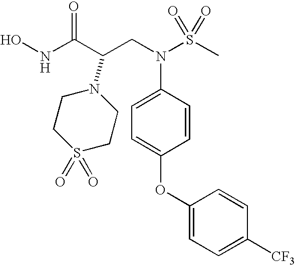 | N-Hydroxy (2S)-2-[1,1-bis(oxo)thiomorpholin- 4-yl]-3-{N-[4-(4- trifluoromethylphenoxy)phenyl], N- methanesulfonylamino}propionamide |
| 71 | 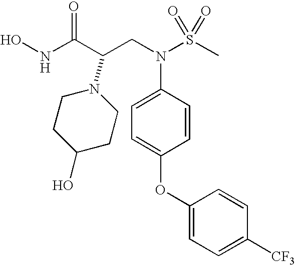 | N-Hydroxy (2S)-2-(4-hydroxypiperidin-1-yl)- 3-{N-[4-(4-trifluoromethylphenoxy)phenyl], N- methanesulfonylamino}propionamide |
| 72 | 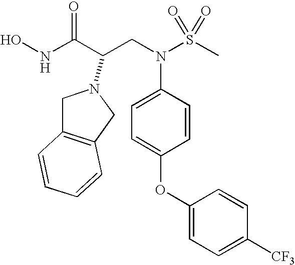 | N-Hydroxy (2S)-2-(isoindolin-2-yl)-3-{N-[4-(4- trifluoromethylphenoxy)phenyl], N- methanesulfonylamino}propionamide |
| 73 | 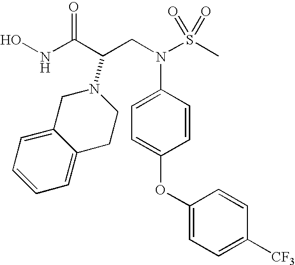 | N-Hydroxy (2S)-2-(1,2,3,4- tetrahydroisoquinolin-2-yl)-3-{N-[4-(4- trifluoromethylphenoxy)phenyl], N- methanesulfonylamino}propionamide |
| 74 | 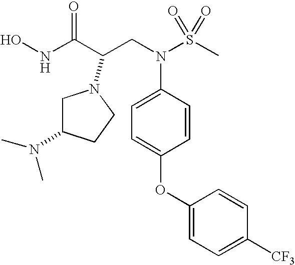 | N-Hydroxy (2S)-2-[(3S)-3-N,N- dimethylaminopyrrolidin-1-yl]-3-{N-[4-(4- trifluoromethylphenoxy)phenyl], N- methanesulfonylamino}propionamide |
| 75 | 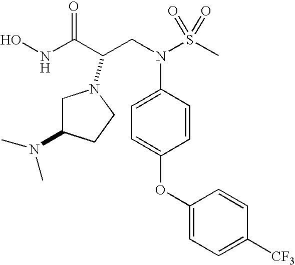 | N-Hydroxy (2S)-2-[(3R)-3-N,N- dimethylaminopyrrolidin-1-yl]-3-{N-[4-(4- trifluoromethylphenoxy)phenyl], N- methanesulfonylamino}propionamide |
| 76 | 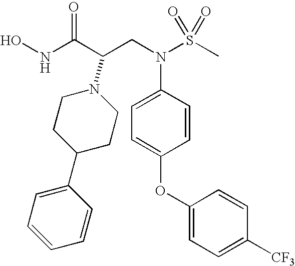 | N-Hydroxy (2S)-3-{N-[4-(4- trifluoromethylphenoxy)phenyl], N- methanesulfonylamino}-2-(4-phenylpiperidin- 1-yl)propionamide |
| 77 | 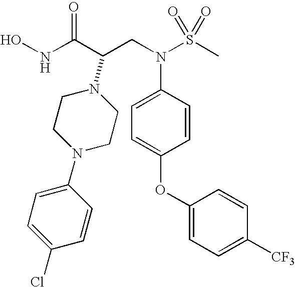 | N-Hydroxy (2S)-2-[4-(4- chlorophenyl)piperazin-1-yl]-3-{N-[4-(4- trifluoromethylphenoxy)phenyl], N- methanesulfonylamino}propionamide |
| 78 | 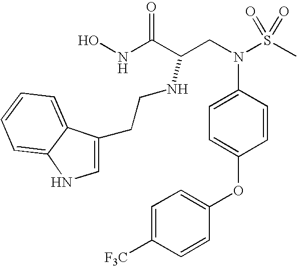 | N-Hydroxy (2S)-2-[(2-(indol-3-yl)ethylamino]- 3-{N-[4-(4-trifluoromethylphenoxy)phenyl], N- methanesulfonylamino}propionamide |
| 79 | 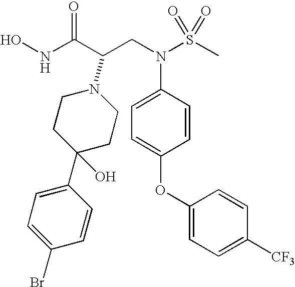 | N-Hydroxy (2S)-2-[4-(4-bromophenyl)-4- hydroxypiperidin-1-yl]-3-{N-[4-(4- trifluoromethylphenoxy)phenyl], N- methanesulfonylamino}propionamide |
| 80 | 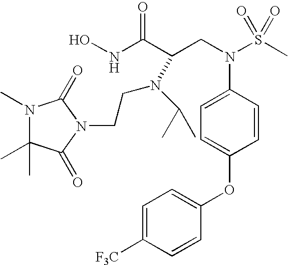 | N-Hydroxy (2S)-2-{N-(2-propyl), N-[2-(1,5,5- trimethylhydantoin-3-yl)ethyl]amino}-3-{N-[4- (4-trifluoromethylphenoxy)phenyl]-N- methanesulfonylamino}propionamide |
| 81 | 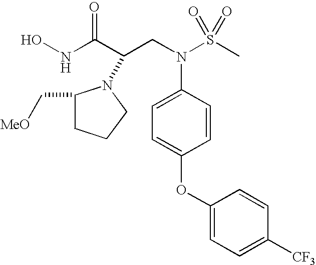 | N-Hydroxy (2S)-2-[(2R)-2- methoxymethylpyrrolidin-1-yl]-3-{N-[4-(4- trifluoromethylphenoxy)phenyl], N- methanesulfonylamino}propionamide |
| 82 | 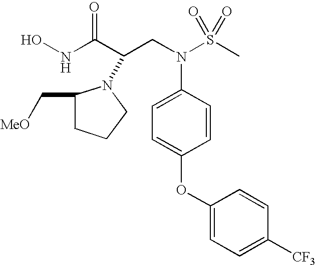 | N-Hydroxy (2S)-2-[(2S)-2- methoxymethylpyrrolidin-1-yl]-3-{N-[4-(4- trifluoromethylphenoxy)phenyl], N- methanesulfonylamino}propionamide |
| 83 | 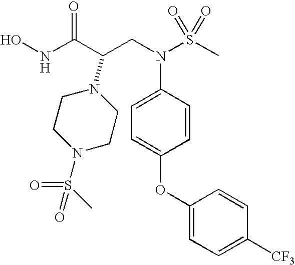 | N-Hydroxy (2S)-2-(4- methanesulfonylpiperazin-1-yl)-3-{N-[4-(4- trifluoromethylphenoxy)phenyl], N- methanesulfonylamino}propionamide |
| 84 | 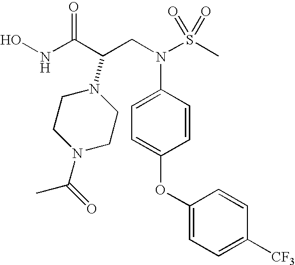 | N-Hydroxy (2S)-2-(4-acetylpiperazin-1-yl)-3- {N-[4-(4-trifluoromethylphenoxy)phenyl], N- methanesulfonylamino}propionamide |
| 85 | 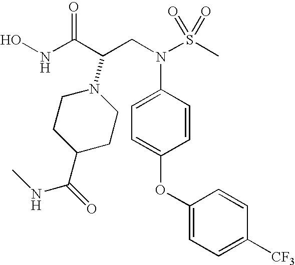 | N-Hydroxy (2S)-2-[4-(N- methylaminocarbonyl)piperidin-1-yl]-3-{N-[4- (4-trifluoromethylphenoxy)phenyl], N- methanesulfonylamino}propionamide |
| 86 | 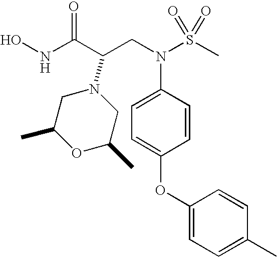 | N-Hydroxy (2S)-2-(cis-2,6- dimethylmorpholin-4-yl)-3-{N-[4-(4- trifluoromethylphenoxy)phenyl], N- methanesulfonylamino}propionamide |
| 87 | 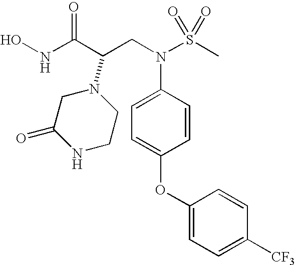 | N-Hydroxy (2S)-2-(3-oxopiperazin-1-yl)-3-{N- [4-(4-trifluoromethylphenoxy)phenyl], N- methanesulfonylamino}propionamide |
| 88 | 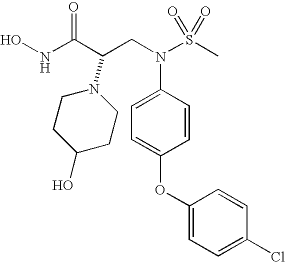 | N-Hydroxy (2S)-3-{N-[4-(4- chlorophenoxy)phenyl], N- methanesulfonylamino}-2-(4- hydroxypiperidin-1-yl)propionamide |
| 89 | 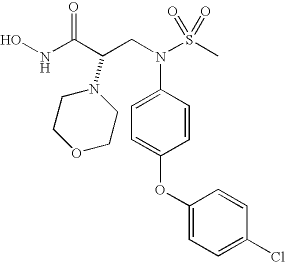 | N-Hydroxy (2S)-2-(morpholin-4-yl)-3-{N-[4- (4-chlorophenoxy)phenyl], N- methanesulfonylamino}propionamide |
| 90 | 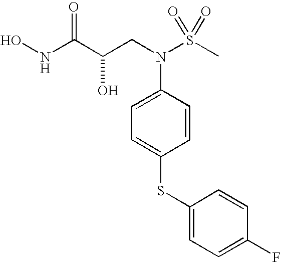 | N-Hydroxy (2S)-2-hydroxy-3-{N-[4-(4- fluorothiophenoxy)phenyl], N- methanesulfonylamino}propionamide |
| 91 | 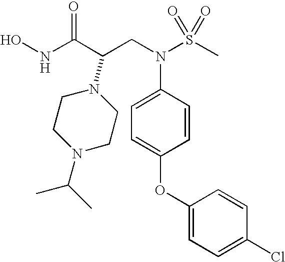 | N-Hydroxy (2S)-3-{N-[4-(4- chlorophenoxy)phenyl], N- methanesulfonylamino}-2-[4-(2- propyl)piperazin-1-yl]propionamide |
| 92 | 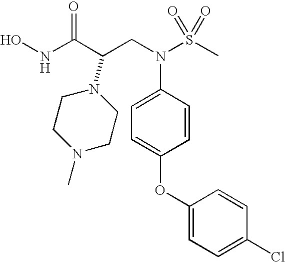 | N-Hydroxy (2S)-3-{N-[4-(4- chlorophenoxy)phenyl], N- methanesulfonylamino}-2-(4- methylpiperazin-1-yl)propionamide |
| 93 | 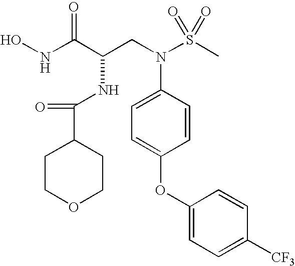 | N-Hydroxy (2S)-2-[(tetrahydropyran-4- yl)carbonylamino]-3-{N-[4-(4- trifluoromethylphenoxy)phenyl], N- methanesulfonylamino}propionamide |
| 94 | 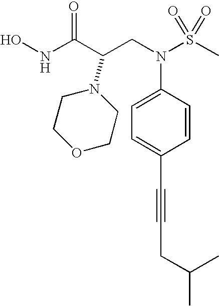 | N-Hydroxy (2S)-3-{N-[4-(4-methylpentyn-1- yl)phenyl], N-methanesulfonylamino}-2- (morpholin-4-yl)propionamide |
| 95 | 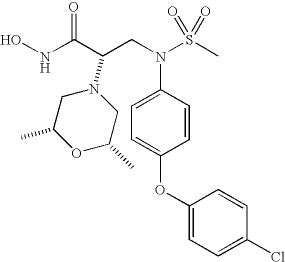 | N-Hydroxy (2S)-3-{N-[4-(4- chlorophenoxy)phenyl], N- methanesulfonylamino}-2-(cis-2,6- dimethylmorpholin-4-yl)propionamide |
| 96 | 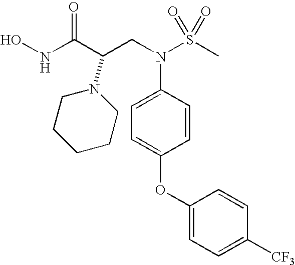 | N-Hydroxy (2S)-2-(piperidin-1-yl)-3-{N-[4-(4- trifluoromethylphenoxy)phenyl], N- methanesulfonylamino}propionamide |
| 97 | 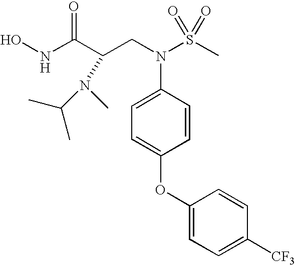 | N-Hydroxy (2S)-2-[N-methyl, N-(2- propyl)amino]-3-{N-[4-(4- trifluoromethylphenoxy)phenyl], N- methanesulfonylamino}propionamide |
| 98 | 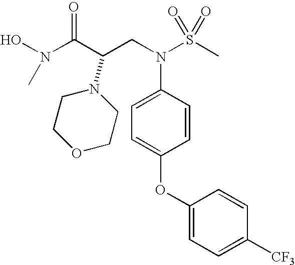 | N-Hydroxy, N-methyl (2S)-2-(morpholin-4-yl)- 3-{N-[4-(4-trifluoromethylphenoxy)phenyl], N- methanesulfonylamino}propionamide |
| 99 | 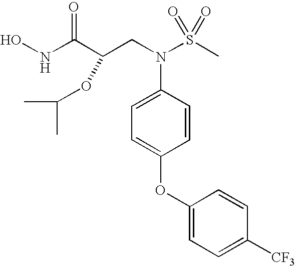 | N-Hydroxy (2S)-2-(2-propoxy)-3-{N-[4-(4- trifluoromethylphenoxy)phenyl], N- methanesulfonylamino}propionamide |
| 100 | 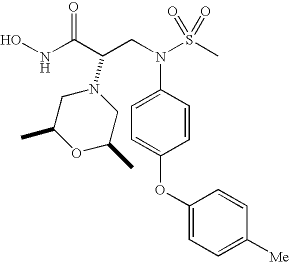 | N-Hydroxy (2S)-2-(cis-2,6- dimethylmorpholin-4-yl)-3-{N-[4-(4- methylphenoxy)phenyl], N- methanesulfonylamino}propionamide |
| 101 | 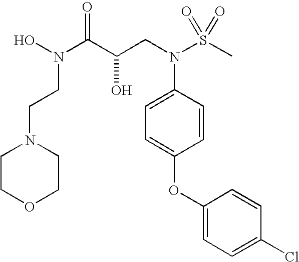 | N-Hydroxy, N-[2-(morpholin-4-yl)ethyl] (2S)-2- hydroxy-3-{N-[4-(4-chlorophenoxy)phenyl], N- methanesulfonylamino}propionamide |
| 102 | 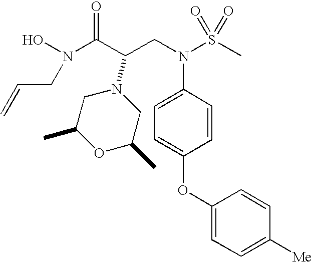 | N-(3-Allyl), N-hydroxy (2S)-2-(cis-2,6- dimethylmorpholin-4-yl)-3-{N-[4-(4- methylphenoxy)phenyl], N- methanesulfonylamino}propionamide |

and solvates, hydrates, tautomers and pharmaceutically acceptable salts thereof.

7. A compound which is selected from the group consisting of 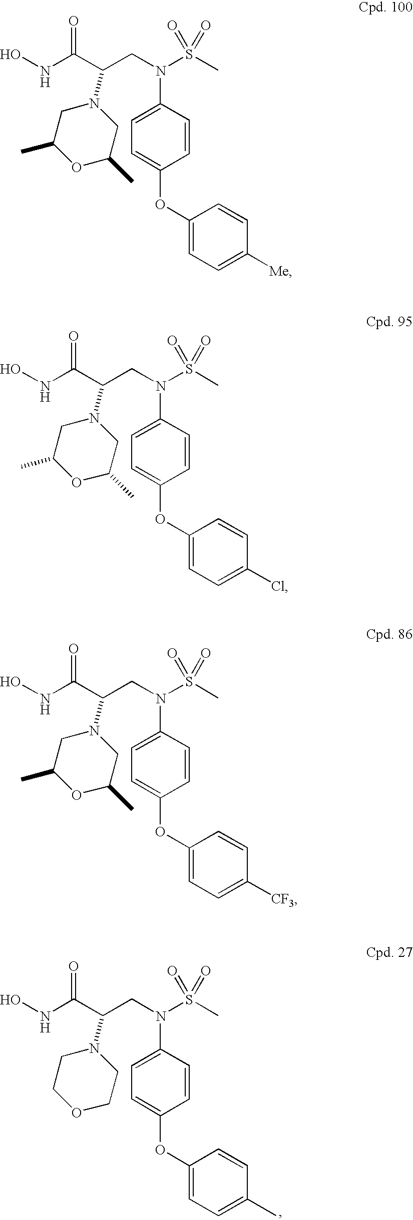 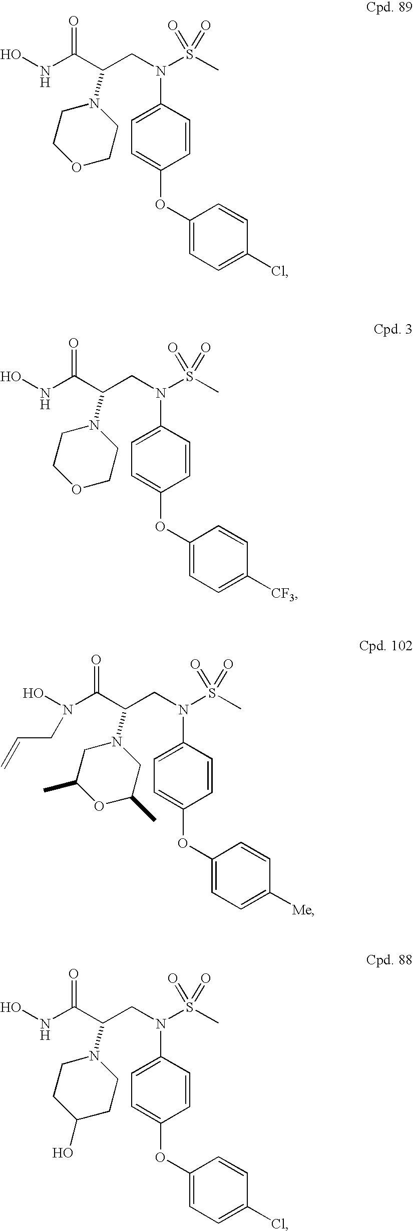 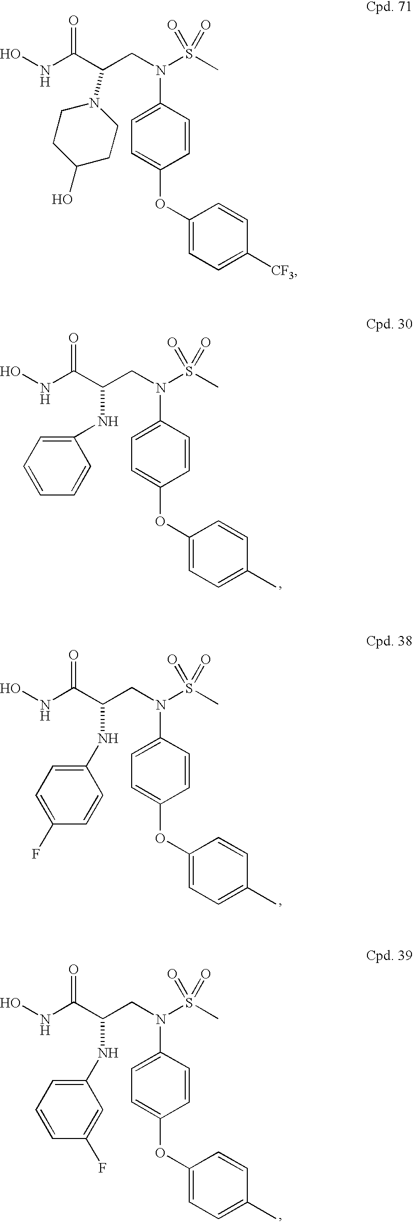
and solvates, hydrates, tautomers and pharmaceutically acceptable salts thereof.

8. A compound which is 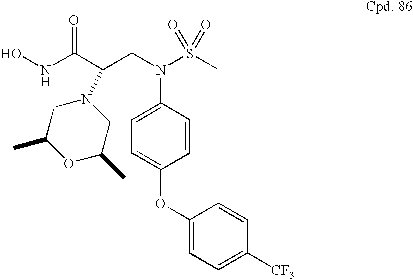
and solvates, hydrates, tautomers and pharmaceutically acceptable salts thereof.

9. A compound which is 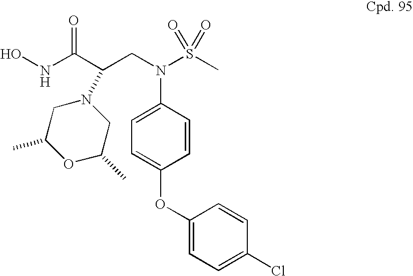
and solvates, hydrates, tautomers and pharmaceutically acceptable salts thereof.

10. A compound which is 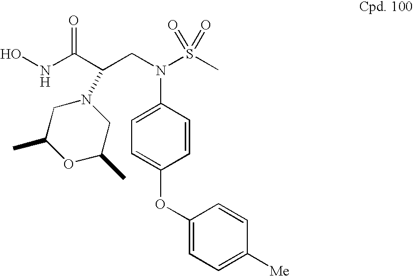
and solvates, hydrates, tautomers and pharmaceutically acceptable salts thereof.

11. A pharmaceutical composition comprising a compound of claims 1-10 and a pharmaceutically acceptable carrier.

12. A compound as claimed in any of claims 1 to 10 for use as a medicine.

13. Use of a compound as claimed in any of claims 1 to 10 for the manufacture of a medicament for the treatment of a cell proliferative disorder.

14. A method for reducing matrix metalloprotease activity in a cell comprising the step of contacting the cell with a compound of claims 1-10.

15. A method for inhibiting matrix metalloprotease activity in a cell comprising the step of contacting the cell with a compound of claims 1-10.

16. A method for reducing matrix metalloprotease activity in a subject comprising the step of administering a compound of claims 1-10 to the subject.

17. A method for inhibiting matrix metalloprotease activity in a subject comprising the step of administering a compound of claims 1-10 to the subject.

18. A method for preventing in a subject a disorder related to matrix metalloprotease activity comprising administering to the subject a prophylactically effective amount of a pharmaceutical composition comprising a compound of claims 1-10 and a pharmaceutically acceptable carrier.

19. A method of treating in a subject a disorder related to matrix metalloprotease activity comprising administering to the subject a therapeutically effective amount of a pharmaceutical composition comprising a compound of claims 1-10 and a pharmaceutically acceptable carrier.

20. A method for the treatment of stroke comprising administering to the subject a therapeutically effective amount of a pharmaceutical composition comprising a compound of claims 1-10 and a pharmaceutically acceptable carrier.
